SEQUENCE LISTING (2 sequences)

SEQ ID NO: 1 - DNA, 80 bp, synthetic construct
  source 1..80: mol_type = other DNA
ttatatcctttactttgaattctatgtttaaccttttacttattttgtattagccggatccttatttcaattatgttcat

SEQ ID NO: 2 - DNA, 80 bp, synthetic construct
  source 1..80: mol_type = other DNA
atgaacataattgaaataaggatccggctaatacaaaataagtaaaaggttaaacatagaattcaaagtaaaggatataa

(19) United States
(12) Patent Application Publication
(10) Pub. No.: US 2025/0009750 A1
(43) Pub. Date: Jan. 9, 2025

(54) METHODS OF TREATING DISORDERS ASSOCIATED WITH EYA OVEREXPRESSION AND MUTATIONS

(71) Applicant: The Board of Regents of the University of Texas System, Austin, TX (US)

(72) Inventors: Eloise Dray, Boerne, TX (US); Stanton F. McHardy, San Antonio, TX (US)

(21) Appl. No.: 18/666,636

(22) Filed: May 16, 2024

Related U.S. Application Data
(60) Provisional application No. 63/502,876, filed on May 17, 2023.

(51) Int. Cl. A61K 31/517; A61P 35/00

(52) U.S. Cl. CPC A61K 31/517; A61P 35/00

(57) ABSTRACT

The present disclosure is concerned with methods of treating disorders associated with overexpression of an eyes absent (EYA) protein such as, for example, vascular disease, a fibrosis-related disorder, hearing loss, and a metabolic disease using quinazoline-2,4-diamines. Also disclosed are methods of treating cancer that comprises a tumor that overexpresses at least one eyes absent (EYA) protein (e.g., breast cancer, cervical cancer, ovarian cancer, liver cancer, pancreatic cancer, pediatric cancers). This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/502,876, filed on May 17, 2023, the contents of which are hereby incorporated by reference in their entirety.

### REFERENCE TO SEQUENCE LISTING

The Sequence Listing submitted Sep. 17, 2024 as a xml file named “21105.0094U2a.xml,” created on May 16, 2024, and having a size of 4,096 bytes is hereby incorporated by reference pursuant to 37 C.F.R. § 1.52(e)(5).

### BACKGROUND

Kinases are enzymes responsible for the addition of phosphate groups onto proteins, while protein phosphatases counteract their action and remove phosphate from phosphoresidues. Phosphorylation of proteins are some of the most frequent and abundant post translational modifications, yet their reversal remains difficult to study. Deep coverage phospho-proteomic studies have shown that at least 75% of the proteins are phosphorylable (Sharma et al. (2014) Cell Reports 8(5): P1583-1594). Three amino-acids account for the majority of phosphorylations: serine, threonine, and tyrosine. While phosphorylation events on serine and threonine are both abundant and relatively stable, phosphorylation of tyrosine residues are rare events, transient, and mostly restricted to well-expressed proteins.

In Eukaryotes, kinases are plentiful and regulate all major biological functions such as DNA replication and cell cycle progression, cell differentiation, hormone signalling, DNA damage repair, and immunity. Hyperactivation of kinases are linked to cancer development and progression, and a variety of kinases inhibitors are currently in clinical trial (such as drugs inhibiting Wee1, tyrosine kinases, or Rho) or already FDA approved. Conversely, protein phosphatases, which remove phosphate from proteins and are thus regulating the same pathways as kinases, are for most part poorly characterized drug targets. As of March 2023, there were 8202 clinical trials investigating kinase inhibitors, and less than 600 for phosphatase inhibitors.

DNA is constantly at risk of damage under the pressure of exogenous and endogenous genotoxic agents. Among all breaks and lesions that occur during each cell cycle, DNA double-strand breaks (DSBs) are arguably the most deleterious. If not properly repaired, these can lead to extensive genomic rearrangement, chromosome fragmentation, or even chromosome loss. All living organisms have evolved complex mechanisms to repair DNA damage and preserve genomic integrity. In higher eukaryotes, the two major pathways for DSB repair are non-homologous end joining (NHEJ) and homologous recombination (HR). While NHEJ ligates the two broken extremities of DNA with minimal processing of the DNA ends, HR is a more faithful mechanism that relies on extensive regions of homology between the broken DNA and its repair template to restore an intact DNA copy at the break site. This involves extensive resection of the DSB to free a ssDNA that will be coated by RAD51 and used to carry homology search.

Major players of both pathways, which compete to repair DSBs, are well characterized (J. M. Daley, P. Sung, Mol Cell Biol 34, 1380-1388 (2014), T. T. Paull, Curr Opin Genet Dev 71, 55-62 (2021)). Their tight link with cell-cycle progression ensures that resection and thus HR is only initiated in S or G2 when a sister chromatid is present; and prevents the formation of potentially deleterious structures that would impair recombination and ligation (D. Wang et al., Sci Adv 7 (2021), Z. Mirman, T. de Lange, Genes Dev 34, 7-23 (2020), K. Rothkamm et al., Environ Mol Mutagen 56, 491-504 (2015)). Following a DNA break, cell-cycle-regulated kinases such as ATM and ATR sense the damage and phosphorylate DDR effectors. Many DDR proteins relocate to chromatin upon phosphorylation, including the recombinase RAD51 (P. Moudry et al., J Cell Biol 212, 281-288 (2016)). The chromatin environment of the break, including the phosphorylation status of the histone variant H2AX, plays a major role in DNA repair processes (T. Clouaire et al., Mol Cell 72, 250-262 e256 (2018)). One of the earliest phosphorylation events in the cascade that activates DDR machinery is that of H2AX on serine 139 (I. Revet et al., Proc Natl Acad Sci U.S.A 108, 8663-8667 (2011)), which activates it into γH2AX. γH2AX acts as a beacon for DSB repair, recruiting DDR factors to converge at the break site. Histones can be heavily modified on their flexible tails, and subunits of the nucleosome can be substituted to incorporate histone variants specific to DNA replication or repair.

Post-translational modifications of histones can also signal overwhelming DNA damage and direct cells toward apoptosis rather than DNA repair. While phosphorylation of H2AX at S139 activates it into γH2AX and promotes DNA repair, the nearby residue Y142 must be dephosphorylated for repair to take place. Concomitant phosphorylation of S139 and Y142 is associated with apoptosis (J. A. Brown, J. K. FEBS Open Bio 2, 313-317 (2012), P. J. Cook et al., Nature 458, 591-596 (2009)).

Three phosphatases from the Eyes Absent family (EYA1/2/3) have been shown to dephosphorylate H2AX pY142 in response to DSBs and the fourth member, EYA4, was proposed by similarity to perform the same function. EYA4 contains a compact serine/threonine phosphatase domain (residues 268-292) and a larger tyrosine phosphatase domain that contains 80 amino acids, important to the catalytic activity of EYA4, and fragmented into four individual motifs (spanning residues 369-614) (FIG. 1A). Both domains are well conserved across vertebrates (FIG. 1A) although not typical of phosphatase domains. EYA4 is an atypical phosphatase (R. S. Hegde, K. Roychoudhury, R. N. Pandey, Crit Rev Biochem Mol Biol 55, 372-385 (2020)) in that its two catalytic domains contain acidic residues.

EYA proteins are transcriptional coactivators that control the development and maintenance of the eye and the cochlear organ of Corti. Defects in EYA proteins have been associated with neural defects, deafness (T. Ishino et al., Otol Neurotol 42, e866-e874 (2021), M. Morin et al., Sci Rep 10, 6213 (2020), and J. Shinagawa et al., Sci Rep 10, 3662 (2020)), and cardiomyopathies (S. Ahmadmehrabi et al., Hum Genet 140, 957-967 (2021) and Y. Mi et al., Mol Genet Genomic Med 9, e1569 (2021)). EYA4 deficiencies have also been associated with lung carcinogenesis (I. M. Wilson et al., Oncogene 33, 4464-4473 (2014)) and oral dysplasia (R. Towle, D. Truong, C. Garnis, Genes Chromosomes Cancer 55, 568-576 (2016)). EYA4 is the least well-characterized member of the family, and only a few targets have been identified as EYA4 substrates. By similarity, ERb (pY36), H2AX (pY142), and WDR1 (pY238) are all potential targets of the tyrosine phosphatase activity of EYA4 (I. Rebay, Mol Cell Biol 36, 668-677 (2015), H. Zhou, L. Zhang, R. L. Vartuli, H. L. Ford, R. Zhao, Int J Biochem Cell Biol 96, 165-170 (2018), and B. Yuan et al., The Journal of clinical investigation 124, 3378-3390 (2014)).

Despite the known correlation between defects in EYA proteins and a variety of different disorders (e.g., vascular diseases, fibrosis-related disorders, hearing loss, metabolic diseases, cancer), the identification of potent, selective inhibitors of these proteins has remained elusive. Thus, there is a need for compounds and compositions for treating disorders associated with overexpression of EYA proteins (e.g., EYA2, EYA4) and methods of making and using same.

### SUMMARY

In accordance with the purpose(s) of the invention, as embodied and broadly described herein, the invention, in one aspect, relates to substituted quinazoline-2,4-diamines and compositions for use in treating disorders associated with overexpression of EYA proteins such as, for example, vascular diseases, fibrosis-related disorder, hearing loss, a metabolic disease, and cancer that comprises a tumor that overexpresses at least one EYA protein or expresses a dysfunctional, mutated or truncated version of at least one EYA protein (e.g., breast cancer, triple-negative breast cancer (TNBC), cervical cancer, ovarian cancer, liver cancer, pancreatic cancer, pediatric cancer, or leukemia).

Thus, disclosed are methods of treating a disorder associated with overexpression of an eyes absent (EYA) protein in a subject in need thereof, the method comprising administering to the subject an effective amount of a compound having a structure represented by a formula:

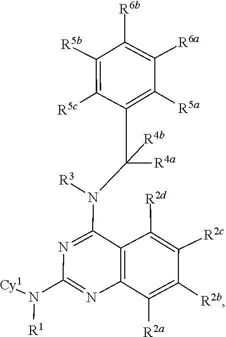

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{6a} and R^{6b} is independently selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least one of R^{6a} and R^{6b} is halogen, or a pharmaceutically acceptable salt thereof, wherein the disorder is a vascular disease, a fibrosis-related disorder, hearing loss, or a metabolic disease.

Also disclosed are methods of treating cancer in a subject in need thereof, the method comprising administering to the subject a compound having a structure represented by a formula:

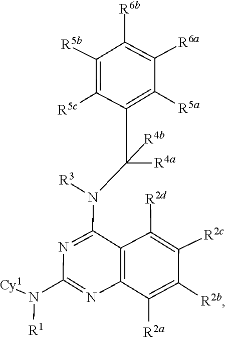

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{6a} and R^{6b} is independently selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least one of R^{6a} and R^{6b} is halogen, or a pharmaceutically acceptable salt thereof, wherein the cancer comprises a tumor that overexpresses at least one eyes absent (EYA) protein.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings.

FIG. 1A-E show representative data illustrating that atypical protein phosphatase EYA4 depletion in cell lines causes genomic instability.

FIG. 2A-F show representative data illustrating that EYA4 contributes to DNA repair signaling by dephosphorylating H2AX residue Y142.

FIG. 3A-F show representative data illustrating that EYA4 is a DNA and histones binding protein.

FIG. 4A-E show representative data illustrating that EYA4 takes part in DNA repair foci assembly.

FIG. 5A-D show representative data illustrating that EYA4 is essential for efficient homologous recombination repair of double strand breaks.

FIG. 6A-H show representative data illustrating that the phosphorylation status of RAD51 controls its activity.

FIG. 7A and FIG. 7B show representative data illustrating the time course of pH2AX (Y142) accumulation in response to DNA damage.

FIG. 8A-D show representative data illustrating that EYA4 is a DNA binding protein.

FIG. 9A and FIG. 9B show representative data illustrating that EYA4 depleted cells exhibit limited resection.

FIG. 10A and FIG. 10B show representative data illustrating that EYA4 depleted cells fail to recruit RAD51 at DSBs.

FIG. 11A-D show representative data illustrating the characteristics of RAD51 WT and mutants.

FIG. 12A and FIG. 12B show representative models for EYA4 role.

FIG. 13A and FIG. 13B show show representative data that EYA tyrosine phosphatase inhibition decreases homologous recombination efficiency without impacting single strand annealing.

FIG. 14 shows representative data illustrating that an exemplary compound efficiently inhibits tyrosine phosphatase in the micromolar concentration range, and efficiently suppress cell growth in cells expressing high levels of EYA4.

FIG. 15A and FIG. 15B show representative data that EYA4 phosphatase inhibition prevents completion of the cell cycle.

FIG. 16A-D show representative data illustrating the dose-dependent accumulation of EYA4 cells in S-phase in response to treatment.

FIG. 17A-H show representative data illustrating that EYA4 is a novel breast cancer oncogene.

FIG. 18A-K show representative data illustrating that EYA4 promotes breast cancer development through its serine/threonine phosphatase domain.

FIG. 19A-D show representative data illustrating that EYA4 regulates proliferation and migration in cells.

FIG. 20A-E show representative data illustrating that EYA4 disturbs cell cycle progression and triggers cell cycle checkpoints.

FIG. 21A-C show representative data illustrating that EYA4-depleted cells accumulate spontaneous replication stress.

FIG. 22A-F show representative data illustrating the impact of EYA4 on replication fork progression.

FIG. 23A-E show representative data illustrating expression pattern of eyes absent 4 (EYA4) in breast cancer.

FIG. 24A-F show representative data illustrating that the serine/threonine phosphatase domain of EYA4 is essential for breast cancer progression.

FIG. 25A-M show representative data illustrating that EYA4 regulates proliferation and migration in cells.

FIG. 26A-C show representative raw data of cell cycle analysis using FlowJo.

FIG. 27A-C show representative schematics and data illustrating EYA4 cell cycle progression monitored by live imaging utilizing the FUCCI system.

FIG. 28 shows representative data illustrating that EYA4 is overexpressed in several (solid-tumor) pediatric cancers.

FIG. 29 shows representative data illustrating that EYA4 is frequently found rearranged as oncogenic fusions in pediatric tumors.

FIG. 30A and FIG. 30B show representative data illustrating a pathway analysis of the shared interactome between EWSR1: EYA4 and EWS::FL1: EYA4.

FIG. 31 shows representative image of colocalization of EYA4 and CTD.

FIG. 32A and FIG. 32B show representative data that EYA4i inhibits EYA tyrosine phosphatase and promotes killing of EYA>0 EwS cells.

FIG. 33 shows representative image of EYA4 being efficient as a single agent in pediatric cancer cell lines CHL1 and EW8.

FIG. 34A-C show representative data of survival of treated HRD+ pancreatic cell lines.

FIG. 35 shows representative data indicating that CPT and PARPi are not lethal.

FIG. 36 shows representative data indicating that EYA4i is efficient as a single agent but also sensitizes cells to replication poisons in TKCC02.1 cells.

FIG. 37 shows representative data indicating that EYA4i is efficient as a single agent but also sensitizes cells to replication poisons in TKCC18 cells.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated+10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀,” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease, disorder, or condition. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the condition being treated and the severity of the condition; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, CT-C7 alkyl, CT-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bond, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbomenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bond. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide” as used herein can be used interchangeably and refer to F, Cl, Br, or I.

The terms “pseudohalide,” “pseudohalogen,” or “pseudohalo” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, 0, N, Si, P and S, wherein the nitrogen, phosphorous and sulfur atoms are optionally oxidized, and the nitrogen heteroatom is optionally quaterized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A'S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{∘}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, =NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR*, —O(haloR^{●}), —CN, —C(O)OH, —C(O)OR*, —NH₂, —NHR*, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR*, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

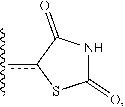

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di-, or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

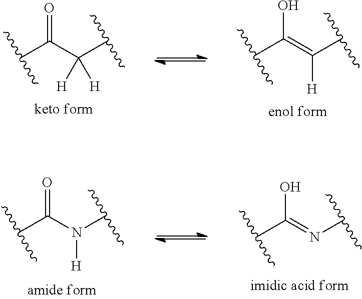 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

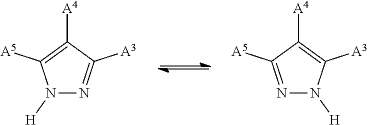

Unless stated to the contrary, the invention includes all such possible tautomers.

It is known that chemical substances form solids, which are present in different states of order, which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

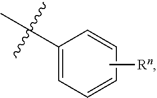

which is understood to be equivalent to a formula:

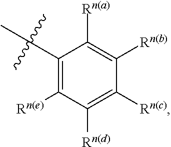

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C—F are considered disclosed. Likewise, any subset or combination of these is also disclosed. Thus, for example, the sub-group of A-E, B—F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

In one aspect, the invention relates to substituted quinazoline-2,4-diamines useful in preventing and treating a disorder associated with overexpression of EYA proteins (e.g., EYA2, EYA4). In a further aspect, the disorder is a vascular disease, a fibrosis-related disorder, hearing loss, or a metabolic disease.

In one aspect, the invention relates to substituted quinazoline-2,4-diamines useful in treating a cancer that comprises a tumor that overexpresses at least one EYA protein. Examples of cancers for which the disclosed compounds can be used include, but are not limited to, breast cancer, cervical cancer, ovarian cancer, liver cancer, and pancreatic cancer. In a further aspect, the cancer is triple-negative breast cancer (TNBC). In a still further aspect, the cancer is a pediatric cancer (e.g., leukemia).

In one aspect, the compounds of the invention are useful in the treatment of a vascular disease, a fibrosis-related disorder, hearing loss, or a metabolic disease, as further described herein.

In one aspect, the compounds of the invention are useful in the treatment of cancer that comprises a tumor that overexpresses at least one EYA protein, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

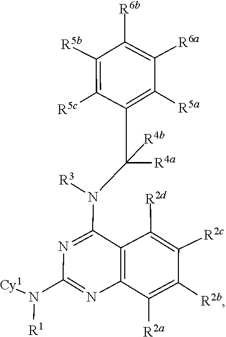

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and W is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{6a} and R^{6b} is independently selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least one of R^{6a} and R^{6b} is halogen, or a pharmaceutically acceptable salt thereof.

In various aspects, the compound is present as a pharmaceutically acceptable salt. Examples of pharmaceutically acceptable salts include, but are not limited to, acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate, and xinofoate salts. In a further aspect, the compound is present as a hydrochloride salt.

In various aspects, the compound has a structure represented by a formula:

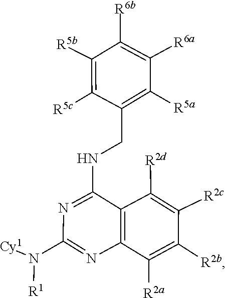

or a pharmaceutically acceptable salt thereof.

In various aspects, the compound has a structure represented by a formula:

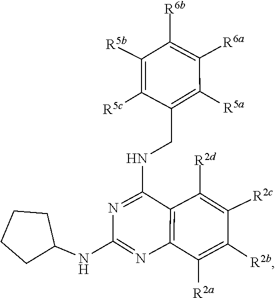

or a pharmaceutically acceptable salt thereof.

In various aspects, the compound has a structure represented by a formula:

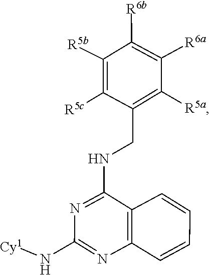

or a pharmaceutically acceptable salt thereof.

In various aspects, the compound has a structure represented by a formula:

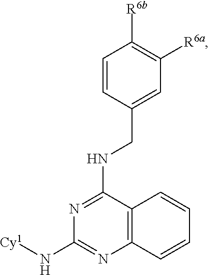

or a pharmaceutically acceptable salt thereof.

In various aspects, the compound has a structure represented by a formula:

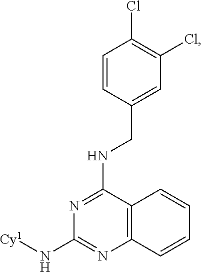

or a pharmaceutically acceptable salt thereof.

In various aspects, the compound is a structure selected from:

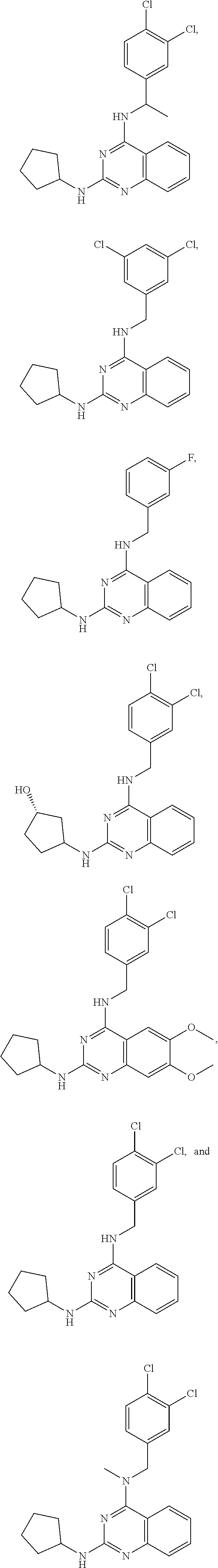

or a pharmaceutically acceptable salt thereof.

In various aspects, the compound is:

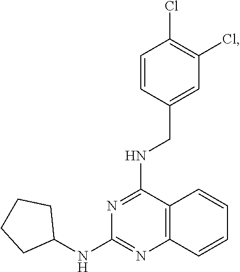 [SMILES: Clc1ccc(CNc2nc(NC3CCCC3)nc3ccccc23)cc1Cl]

or a pharmaceutically acceptable salt thereof.
a. R¹ Groups

In one aspect, R¹ is selected from hydrogen and C1-C4 alkyl. In a further aspect, R¹ is selected from hydrogen, methyl, ethyl, n-propyl, and isopropyl. In a still further aspect, R¹ is selected from hydrogen, methyl, and ethyl. In yet a further aspect, R¹ is selected from hydrogen and ethyl. In an even further aspect, R¹ is selected from hydrogen and methyl.

In various aspects, R¹ is C1-C4 alkyl. In a further aspect, R¹ is selected from methyl, ethyl, n-propyl, and isopropyl. In a still further aspect, R¹ is selected from methyl and ethyl. In yet a further aspect, R¹ is ethyl. In an even further aspect, R¹ is methyl.

In various aspects, R¹ is hydrogen.

b. R^{2A}, R^{2B}, R^{2C}, R^{2D}, and R^{2E} Groups

In one aspect, each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each of R^{2a}, R^{2b}, R^{2c}, R^{2d}, and R^{2e} is independently selected from hydrogen, —F, —Cl, —CN, —NH₂, —OH, —NO₂, methyl, ethyl, n-propyl, isopropyl, ethenyl, propenyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{2a}, R^{2b}, R^{2c}, R^{2d}, and R^{2e} is independently selected from hydrogen, —F, —Cl, —CN, —NH₂, —OH, —NO₂, methyl, ethyl, ethenyl, —CCl₃, —CF₃, —CHCl₂, —CHF₂, —CH₂Cl, —CH₂F, —CH₂CH₂Cl, —CH₂CH₂F, —CH₂CN, —CH₂CH₂CN, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{2a}, R^{2b}, R^{2c}, R^{2d}, and R^{2e} is independently selected from hydrogen, —F, —Cl, —CN, —NH₂, —OH, —NO₂, methyl, —CCl₃, —CF₃, —CHCl₂, —CHF₂, —CH₂Cl, —CH₂F, —CH₂CN, —CH₂OH, —OCCl₃, —OCF₃, —OCHCl₂, —OCHF₂, —OCH₂Cl, —OCH₂F, —OCH₃, —NHCH₃, —N(CH₃)₂, and —CH₂NH₂.

In various aspects, each of R^{2a}, R^{2b}, R^{2c}, R^{2d}, and R^{2e} is independently selected from hydrogen, halogen, —CN, —NH₂, —OH, —NO₂, C1-C4 alkyl, and C2-C4 alkenyl. In a further aspect, each of R^{2a}, R^{2b}, R^{2c}, R^{2d}, and R^{2e} is independently selected from hydrogen, —F, —Cl, —CN, —NH₂, —OH, —NO₂, methyl, ethyl, n-propyl, isopropyl, ethenyl, and propenyl. In a still further aspect, each R^{2a}, R^{2b}, R^{2c}, R^{2d}, and R^{2e} is independently selected from hydrogen, —F, —Cl, —CN, —NH₂, —OH, —NO₂, methyl, ethyl, and ethenyl. In yet a further aspect, each of R^{2a}, R^{2b}, R^{2c}, R^{2d}, and R^{2e} is independently selected from hydrogen, —F, —Cl, —CN, —NH₂, —OH, —NO₂, and methyl.

In various aspects, each of R^{2a}, R^{2b}, R^{2c}, R^{2d}, and R^{2e} is independently selected from hydrogen, C1-C4 alkyl, and C2-C4 alkenyl. In a further aspect, each of R^{2a}, R^{2b}, R^{2c}, R^{2d}, and R^{2e} is independently selected from hydrogen, methyl, ethyl, n-propyl, isopropyl, ethenyl, and propenyl. In a still further aspect, each of R^{2a}, R^{2b}, R^{2c}, R^{2d}, and R^{2e} is independently selected from hydrogen, methyl, ethyl, and ethenyl. In yet a further aspect, each of R^{2a}, R^{2b}, R^{2c}, R^{2d}, and R^{2e} is independently selected from hydrogen and methyl.

In various aspects, each of R^{2a}, R^{2b}, R^{2c}, R^{2d}, and R^{2e} is independently selected from hydrogen, halogen, —CN, —NH₂, —OH, —NO₂, C1-C4 haloalkyl, and C1-C4 cyanoalkyl. In a further aspect, each of R^{2a}, R^{2b}, R^{2c}, R^{2d}, and R^{2e} is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{2a}, R^{2b}, R^{2c}, R^{2d}, and R^{2e} is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{2a}, R^{2b}, R^{2c}, R^{2d}, and R^{2e} is independently selected from hydrogen, —F, —Cl, —CN, —NH₂, —OH, —NO₂, —CCl₃, —CF₃, —CHCl₂, —CHF₂, —CH₂Cl, —CH₂F, and —CH₂CN.

In various aspects, each of R^{2a}, R^{2b}, R^{2c}, R^{2d}, and R^{2e} is independently selected from hydrogen, C1-C4 haloalkyl, and C1-C4 cyanoalkyl. In a further aspect, each of R^{2a}, R^{2b}, R^{2c}, R^{2d}, and R^{2e} is independently selected from hydrogen, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{2a}, R^{2b}, R^{2c}, R^{2d}, and R^{2e} is independently selected from hydrogen, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{2a}, R^{2b}, R^{2c}, R^{2d}, and R^{2e} is independently selected from hydrogen, —CCl₃, —CF₃, —CHCl₂, —CHF₂, —CH₂Cl, —CH₂F, and —CH₂CN.

In various aspects, each of R^{2a}, R^{2b}, R^{2c}, R^{2d}, and R^{2e} is independently selected from hydrogen, halogen, —CN, —NH₂, —OH, —NO₂, C1-C4 hydroxyalkyl, C1-C4 haloalkoxy, and C1-C4 alkoxy. In a further aspect, each of R^{2a}, R^{2b}, R^{2c}, R^{2d}, and R^{2e} is independently selected from hydrogen, —F, —Cl, —CN, —NH₂, —OH, —NO₂, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{2a}, R^{2b}, R^{2c}, R^{2d}, and R^{2e} is independently selected from hydrogen, —F, —Cl, —CN, —NH₂, —OH, —NO₂, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{2a}, R^{2b}, R^{2c}, R^{2d}, and R^{2e} is independently selected from hydrogen, —F, —Cl, —CN, —NH₂, —OH, —NO₂, —CH₂OH, —OCCl₃, —OCF₃, —OCHCl₂, —OCHF₂, —OCH₂Cl, —OCH₂F, and —OCH₃.

In various aspects, each of R^{2a}, R^{2b}, R^{2c}, R^{2d}, and R^{2e} is independently selected from hydrogen, C1-C4 hydroxyalkyl, C1-C4 haloalkoxy, and C1-C4 alkoxy. In a further aspect, each of R^{2a}, R^{2b}, R^{2c}, R^{2d}, and R^{2e} is independently selected from hydrogen, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{2a}, R^{2b}, R^{2c}, R^{2d}, and R^{2e} is independently selected from hydrogen, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{2a}, R^{2b}, R^{2c}, R^{2d}, and R^{2e} is independently selected from hydrogen, —CH₂OH, —OCCl₃, —OCF₃, —OCHCl₂, —OCHF₂, —OCH₂Cl, —OCH₂F, and —OCH₃.

In various aspects, each of R^{2a}, R^{2b}, R^{2c}, R^{2d}, and R^{2e} is independently selected from hydrogen, halogen, —CN, —NH₂, —OH, —NO₂, C1-C4 alkylamino, (C1-C4)(C1-C4) dialkylamino, and C1-C4 aminoalkyl. In a further aspect, each of R^{2a}, R^{2b}, R^{2c}, R^{2d}, and R^{2e} is independently selected from hydrogen, —F, —Cl, —CN, —NH₂, —OH, —NO₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{2a}, R^{2b}, R^{2c}, R^{2d}, and R^{2e} is independently selected from hydrogen, —F, —Cl, —CN, —NH₂, —OH, —NO₂, —NHCH₃, —NHCH₂CH₃, —N(CH₃)₂, —N(CH₃)CH₂CH₃, —CH₂NH₂, —CH₂CH₂NH₂. In yet a further aspect, each of R^{2a}, R^{2b}, R^{2c}, R^{2d}, and R^{2e} is independently selected from hydrogen, —F, —Cl, —CN, —NH₂, —OH, —NO₂, —NHCH₃, —N(CH₃)₂, and —CH₂NH₂.

In various aspects, each of R^{2a}, R^{2b}, R^{2c}, R^{2d}, and R^{2e} is independently selected from hydrogen, C1-C4 alkylamino, (C1-C4)(C1-C4) dialkylamino, and C1-C4 aminoalkyl. In a further aspect, each of R^{2a}, R^{2b}, R^{2c}, R^{2d}, and R^{2e} is independently selected from hydrogen, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{2a}, R^{2b}, R^{2c}R^{2d}, and R^{2e} is independently selected from hydrogen, —NHCH₃, —NHCH₂CH₃, —N(CH₃)₂, —N(CH₃)CH₂CH₃, —CH₂NH₂, and —CH₂CH₂NH₂. In yet a further aspect, each of R^{2a}, R^{2b}, R^{2e}, R^{2d}, and R^{2e} is independently selected from hydrogen, —NHCH₃, —N(CH₃)₂, and —CH₂NH₂.

In various aspects, each of R^{2a}, R^{2b}, R^{2c}, R^{2d}, and R^{2e} is independently selected from hydrogen, halogen, C1-C4 alkyl, C1-C4 haloalkyl, C1-C4 haloalkoxy, and C1-C4 alkoxy. In a further aspect, each of R^{2a}, R^{2b}, R^{2c}, R^{2d}, and R^{2e} is independently selected from hydrogen, —F, —Cl, methyl, ethyl, n-propyl, isopropyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{2a}, R^{2b}, R^{2c}, R^{2d}, and R^{2e} is independently selected from hydrogen, —F, —Cl, methyl, ethyl, —CCl₃, —CF₃, —CHCl₂, —CHF₂, —CH₂Cl, —CH₂F, —CH₂CH₂Cl, —CH₂CH₂F, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{2a}, R^{2b}, R^{2c}, R^{2d}, and R^{2e} is independently selected from hydrogen, —F, —Cl, methyl, —CCl₃, —CF₃, —CHCl₂, —CHF₂, —CH₂Cl, —CH₂F, —OCCl₃, —OCF₃, —OCHCl₂, —OCHF₂, —OCH₂Cl, —OCH₂F, and —OCH₃.

In various aspects, each of R^{2a}, R^{2b}, R^{2c}, R^{2d}, and R^{2e} is independently selected from hydrogen and halogen. In a further aspect, each of R^{2a}, R^{2b}, R^{2c}, R^{2d}, and R^{2e} is independently selected from hydrogen, —F, —Cl, and —Br. In a still further aspect, each of R^{2a}, R^{2b}, R^{2c}, R^{2d}, and R^{2e} is independently selected from hydrogen, —F, and —Cl. In yet a further aspect, each of R^{2a}, R^{2b}, R^{2c}, R^{2d}, and R^{2e} is independently selected from hydrogen and —F. In an even further aspect, each of R^{2a}, R^{2b}, R^{2c}, R^{2d}, and R^{2e} is independently selected from hydrogen and —Cl.

In various aspects, each of R^{2a}, R^{2b}, R^{2c}, R^{2d}, and R^{2e} is hydrogen. In a further aspect, at least one of R^{2a}, R^{2b}, R^{2c}, R^{2d}, and R^{2e} is hydrogen. In a still further aspect, two of R^{2a}, R^{2b}, R^{2c}, R^{2d}, and R^{2e} is hydrogen. In yet further aspect, three of R^{2a}, R^{2b}, R^{2c}, R^{2d}, and R^{2e} is hydrogen. In an even further aspect, four of R^{2a}, R^{2b}, R^{2c}, R^{2d}, and R²⁰ is hydrogen.

c. R³ Groups

In one aspect, R³ is selected from hydrogen and C1-C4 alkyl. In a further aspect, R³ is selected from hydrogen, methyl, ethyl, n-propyl, and isopropyl. In a still further aspect, R³ is selected from hydrogen, methyl, and ethyl. In yet a further aspect, R³ is selected from hydrogen and ethyl. In an even further aspect, R³ is selected from hydrogen and methyl.

In various aspects, R³ is C1-C4 alkyl. In a further aspect, R³ is selected from methyl, ethyl, n-propyl, and isopropyl. In a still further aspect, R³ is selected from methyl and ethyl. In yet a further aspect, R³ is ethyl. In an even further aspect, R³ is methyl.

In various aspects, R³ is hydrogen.

d. R^{4A} and R^{4B} Groups

In one aspect, each of R^{4a} and R^{4b} is independently selected from hydrogen and C1-C4 alkyl. In a further aspect, each of R^{4a} and R^{4b} is independently selected from hydrogen, methyl, ethyl, n-propyl, and isopropyl. In a still further aspect, each of R^{4a} and R^{4b} is independently selected from hydrogen, methyl, and ethyl. In yet a further aspect, each of R^{4a} and R^{4b} is independently selected from hydrogen and ethyl. In an even further aspect, each of R^{4a} and R^{4b} is independently selected from hydrogen and methyl.

In various aspects, each of R^{4a} and R^{4b} is independently C1-C4 alkyl. In a further aspect, each of R^{4a} and R^{4b} is independently selected from methyl, ethyl, n-propyl, and isopropyl. In a still further aspect, each of R^{4a} and R^{4b} is independently selected from methyl and ethyl. In yet a further aspect, each of R^{4a} and R^{4b} is independently ethyl. In an even further aspect, each of R^{4a} and R^{4b} is independently methyl.

In various aspects, each of R^{4a} and R^{4b} is independently hydrogen.

e. R^{5A}, R^{5B}, and R^{5C} Groups

In one aspect, each of R^{5a}, R^{5b}, and R^{5c} is independently selected from hydrogen, halogen, —CN, —NH₂, —OH, —N₀₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each of R^{5a}, R^{5b}, and R^{5c} is independently selected from hydrogen, —F, —Cl, —CN, —NH₂, —OH, —NO₂, methyl, ethyl, n-propyl, isopropyl, ethenyl, propenyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{5a}, R^{5b}, and R^{5c} is independently selected from hydrogen, —F, —Cl, —CN, —NH₂, —OH, —N₀₂, methyl, ethyl, ethenyl, —CCl₃, —CF₃, —CHCl₂, —CHF₂, —CH₂Cl, —CH₂F, —CH₂CH₂Cl, —CH₂CH₂F, —CH₂CN, —CH₂CH₂CN, —CH₂OH, —CH₂CH₂OH, —OCC13, —OCF₃, —OCHCl₂, —OCHF₂, —OCH₂Cl, —OCH₂F, —OCH₂CH₂Cl, —OCH₂CH₂F, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{5a}, R^{5b}, and R^{5c} is independently selected from hydrogen, —F, —Cl, —CN, —NH₂, —OH, —NO₂, methyl, —CCl₃, —CF₃, —CHCl₂, —CHF₂, —CH₂Cl, —CH₂F, —CH₂CN, —CH₂OH, —OCCl₃, —OCF₃, —OCHCl₂, —OCHF₂, —OCH₂Cl, —OCH₂F, —OCH₃, —NHCH₃, —N(CH₃)₂, and —CH₂NH₂.

In various aspects, each of R^{5a}, R^{5b}, and R^{5c} is independently selected from hydrogen, halogen, —CN, —NH₂, —OH, —NO₂, C1-C4 alkyl, and C2-C4 alkenyl. In a further aspect, each of R^{5a}, R^{5b}, and R^{5c} is independently selected from hydrogen, —F, —Cl, —CN, —NH₂, —OH, —NO₂, methyl, ethyl, n-propyl, isopropyl, ethenyl, and propenyl. In a still further aspect, each R^{5a}, R^{5b}, and R^{5c} is independently selected from hydrogen, —F, —Cl, —CN, —NH₂, —OH, —NO₂, methyl, ethyl, and ethenyl. In yet a further aspect, each of R^{5a}, R^{5b}, and R^{5c} is independently selected from hydrogen, —F, —Cl, —CN, —NH₂, —OH, —NO₂, and methyl.

In various aspects, each of R^{5a}, R^{5b}, and R^{5c} is independently selected from hydrogen, C1-C4 alkyl, and C2-C4 alkenyl. In a further aspect, each of R^{5a}, R^{5b}, and R^{5c} is independently selected from hydrogen, methyl, ethyl, n-propyl, isopropyl, ethenyl, and propenyl. In a still further aspect, each of R^{5a}, R^{5b}, and R^{5c} is independently selected from hydrogen, methyl, ethyl, and ethenyl. In yet a further aspect, each of R^{5a}, R^{5b}, and R^{5c} is independently selected from hydrogen and methyl.

In various aspects, each of R^{5a}, R^{5b}, and R^{5c} is independently selected from hydrogen, halogen, —CN, —NH₂, —OH, —NO₂, C1-C4 haloalkyl, and C1-C4 cyanoalkyl. In a further aspect, each of R^{5a}, R^{5b}, and R^{5c} is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{5a}, R^{5b}, and R^{5c} is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{5a}, R^{5b}, and R^{5c} is independently selected from hydrogen, —F, —Cl, —CN, —NH₂, —OH, —NO₂, —CCl₃, —CF₃, —CHCl₂, —CHF₂, —CH₂Cl, —CH₂F, and —CH₂CN.

In various aspects, each of R^{5a}, R^{5b}, and R^{5c} is independently selected from hydrogen, C1-C4 haloalkyl, and C1-C4 cyanoalkyl. In a further aspect, each of R^{5a}, R^{5b}, and R^{5c} is independently selected from hydrogen, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{5a}, R^{5b}, and R^{5c} is independently selected from hydrogen, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{5a}, R^{5b}, and R^{5c} is independently selected from hydrogen, —CCl₃, —CF₃, —CHCl₂, —CHF₂, —CH₂Cl, —CH₂F, and —CH₂CN.

In various aspects, each of R^{5a}, R^{5b}, and R^{5c} is independently selected from hydrogen, halogen, —CN, —NH₂, —OH, —NO₂, C1-C4 hydroxyalkyl, C1-C4 haloalkoxy, and C1-C4 alkoxy. In a further aspect, each of R^{5a}, R^{5b}, and R^{5c} is independently selected from hydrogen, —F, —Cl, —CN, —NH₂, —OH, —NO₂, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{5a}, R^{5b}, and R^{5c} is independently selected from hydrogen, —F, —Cl, —CN, —NH₂, —OH, —NO₂, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{5a}, R^{5b}, and R^{5c} is independently selected from hydrogen, —F, —Cl, —CN, —NH₂, —OH, —NO₂, —CH₂OH, —OCCl₃, —OCF₃, —OCHCl₂, —OCHF₂, —OCH₂Cl, —OCH₂F, and —OCH₃.

In various aspects, each of R^{5a}, R^{5b}, and R¹⁰ is independently selected from hydrogen, C1-C4 hydroxyalkyl, C1-C4 haloalkoxy, and C1-C4 alkoxy. In a further aspect, each of R^{5a}, R^{5b}, and R^{5c} is independently selected from hydrogen, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{5a}, R^{5b}, and R^{5c} is independently selected from hydrogen, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{5a}, R^{5b}, and R^{5c} is independently selected from hydrogen, —CH₂OH, —OCCl₃, —OCF₃, —OCHCl₂, —OCHF₂, —OCH₂Cl, —OCH₂F, and —OCH₃.

In various aspects, each of R^{5a}, R^{5b}, and R^{5c} is independently selected from hydrogen, halogen, —CN, —NH₂, —OH, —NO₂, C1-C4 alkylamino, (C1-C4)(C1-C4) dialkylamino, and C1-C4 aminoalkyl. In a further aspect, each of R^{5a}, R^{5b}, and R^{5c} is independently selected from hydrogen, —F, —Cl, —CN, —NH₂, —OH, —NO₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{5a}, R^{5b}, and R^{5c} is independently selected from hydrogen, —F, —Cl, —CN, —NH₂, —OH, —NO₂, —NHCH₃, —NHCH₂CH₃, —N(CH₃)₂, —N(CH₃)CH₂CH₃, —CH₂NH₂, —CH₂CH₂NH₂. In yet a further aspect, each of R^{5a}, R^{5b}, and R^{5c} is independently selected from hydrogen, —F, —Cl, —CN, —NH₂, —OH, —NO₂, —NHCH₃, —N(CH₃)₂, and —CH₂NH₂.

In various aspects, each of R^{5a}, R^{5b}, and R^{5c} is independently selected from hydrogen, C1-C4 alkylamino, (C1-C4)(C1-C4) dialkylamino, and C1-C4 aminoalkyl. In a further aspect, each of R^{5a}, R^{5b}, and R^{5c} is independently selected from hydrogen, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{5a}, R^{5b}, and R^{5c} is independently selected from hydrogen, —NHCH₃, —NHCH₂CH₃, —N(CH₃)₂, —N(CH₃)CH₂CH₃, —CH₂NH₂, and —CH₂CH₂NH₂. In yet a further aspect, each of R^{5a}, R^{5b} and R^{5c} is independently selected from hydrogen, —NHCH₃, —N(CH₃)₂, and —CH₂NH₂.

In various aspects, each of R^{5a}, R^{5b}, and R^{5c} is independently selected from hydrogen, halogen, C1-C4 alkyl, C1-C4 haloalkyl, C1-C4 haloalkoxy, and C1-C4 alkoxy. In a further aspect, each of R^{5a}, R^{5b}, and R^{5c} is independently selected from hydrogen, —F, —Cl, methyl, ethyl, n-propyl, isopropyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{5a}, R^{5b}, and R^{5c} is independently selected from hydrogen, —F, —Cl, methyl, ethyl, —CCl₃, —CF₃, —CHCl₂, —CHF₂, —CH₂Cl, —CH₂F, —CH₂CH₂Cl, —CH₂CH₂F, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{5a}, R^{5b}, and R^{5c} is independently selected from hydrogen, —F, —Cl, methyl, —CCl₃, —CF₃, —CHCl₂, —CHF₂, —CH₂Cl, —CH₂F, —OCC13, —OCF₃, —OCHCl₂, —OCHF₂, —OCH₂Cl, —OCH₂F, and —OCH₃.

In various aspects, each of R^{5a}, R^{5b}, and R^{5c} is independently selected from hydrogen and halogen. In a further aspect, each of R^{5a}, R^{5b}, and R^{5c} is independently selected from hydrogen, —F, —Cl, and —Br. In a still further aspect, each of R^{5a}, R^{5b}, and R^{5c} is independently selected from hydrogen, —F, and —Cl. In yet a further aspect, each of R^{5a}, R^{5b}, and R^{5c} is independently selected from hydrogen and —F. In an even further aspect, each of R^{5a}, R^{5b}, and R^{5c} is independently selected from hydrogen and —Cl.

In various aspects, each of R^{5a}, R^{5b}, and R^{5c} is hydrogen. In a further aspect, at least one of R^{5a}, R^{5b}, and R^{5c} is hydrogen. In a still further aspect, two of R^{5a}, R^{5b}, and R^{5c} is hydrogen.

f. R^{6A} and R^{6B} Groups

In one aspect, R^{6a} is halogen and R^{6b} is selected from hydrogen and halogen.

In one aspect, each of R^{6a} and R^{6b} is independently selected from hydrogen and halogen. In a further aspect, each of R^{6a} and R^{6b} is independently selected from hydrogen, fluoro, chloro, and bromo. In a still further aspect, each of R^{6a} and R^{6b} is independently selected from hydrogen, fluoro, and chloro. In yet a further aspect, each of R^{6a} and R^{6b} is independently selected from hydrogen and fluoro. In an even further aspect, each of R^{6a} and R^{6b} is independently selected from hydrogen and chloro.

In various aspects, R^{6a} is halogen. In a further aspect, R^{6a} is selected from fluoro, chloro, and bromo. In a still further aspect, R^{6a} is selected from fluoro, chloro, and iodo. In yet a further aspect, R^{6a} is selected from fluoro, bromo, and iodo. In an even further aspect, R^{6a} is selected from chloro, bromo, and iodo. In a still further aspect, R^{6a} is selected from fluoro and chloro. In yet a further aspect, R^{6a} is selected from fluoro and bromo. In an even further aspect, R^{6a} is selected from fluoro and iodo. In a still further aspect, R^{6a} is selected from chloro and bromo. In yet a further aspect, R^{6a} is selected from chloro and iodo. In an even further aspect, R^{6a} is selected from bromo and iodo. In a still further aspect, R^{6a} is iodo. In yet a further aspect, R^{6a} is bromo. In an even further aspect, R^{6a} is chloro. In a still further aspect, R^{6a} is fluoro.

In various aspects, R^{6b} is selected from hydrogen and halogen. In a further aspect, R^{6b} is selected from hydrogen, fluoro, chloro, and bromo. In a still further aspect, R^{6b} is selected from hydrogen, fluoro, and chloro. In yet a further aspect, R^{6b} is selected from hydrogen and fluoro. In an even further aspect, R^{6b} is selected from hydrogen and chloro.

In various aspects, R^{6b} is halogen. In a further aspect, R^{6b} is selected from fluoro, chloro, and bromo. In a still further aspect, R^{6b} is selected from fluoro, chloro, and iodo. In yet a further aspect, R^{6b} is selected from fluoro, bromo, and iodo. In an even further aspect, R^{6b} is selected from chloro, bromo, and iodo. In a still further aspect, R^{6b} is selected from fluoro and chloro. In yet a further aspect, R^{6b} is selected from fluoro and bromo. In an even further aspect, R^{6b} is selected from fluoro and iodo. In a still further aspect, R^{6b} is selected from chloro and bromo. In yet a further aspect, R^{6b} is selected from chloro and iodo. In an even further aspect, R^{6b} is selected from bromo and iodo. In a still further aspect, R^{6b} is iodo. In yet a further aspect, R^{6b} is bromo. In an even further aspect, R^{6b} is chloro. In a still further aspect, R^{6b} is fluoro.

In various aspects, R^{6a} is hydrogen.

In various aspects, R^{6b} is hydrogen.

In one aspect, each of R^{6a} and R^{6b} is independently halogen. In a further aspect, each of R^{6a} and R^{6b} is independently selected from fluoro, chloro, and bromo. In a still further aspect, each of R^{6a} and R^{6b} is independently selected from fluoro and chloro. In yet a further aspect, each of R^{6a} and R^{6b} is fluoro. In an even further aspect, each of R^{6a} and R^{6b} is chloro.

g. Cy¹ Groups

In one aspect, Cy¹ is a C3-C8 cycloalkyl substituted with 0, 1, 2, 3, or 4 groups independently selected from halogen, —CN, —NH₂, —OH, —N₀₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy¹ is a C3-C8 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is a C3-C8 cycloalkyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹ is a C3-C8 cycloalkyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹ is a C3-C8 cycloalkyl monosubstituted with a group selected from halogen, —CN, —NH₂, —OH, —N₀₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is an unsubstituted C3-C8 cycloalkyl.

In various aspects, Cy¹ is a C3-C6 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy¹ is a C3-C6 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is a C3-C6 cycloalkyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹ is a C3-C6 cycloalkyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹ is a C3-C6 cycloalkyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is an unsubstituted C3-C6 cycloalkyl.

In various aspects, Cy¹ is a cyclopentyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy¹ is a cyclopentyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is a cyclopentyl substituted with 0, 1, or 2 groups independently selected from halogen, —CN, —NH₂, —OH, —N₀₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹ is a cyclopentyl substituted with 0 or 1 group selected from halogen, —CN, —NH₂, —OH, —N₀₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹ is a cyclopentyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is an unsubstituted cyclopentyl.

### 2. Example Compounds

In one aspect, a compound can be present as:

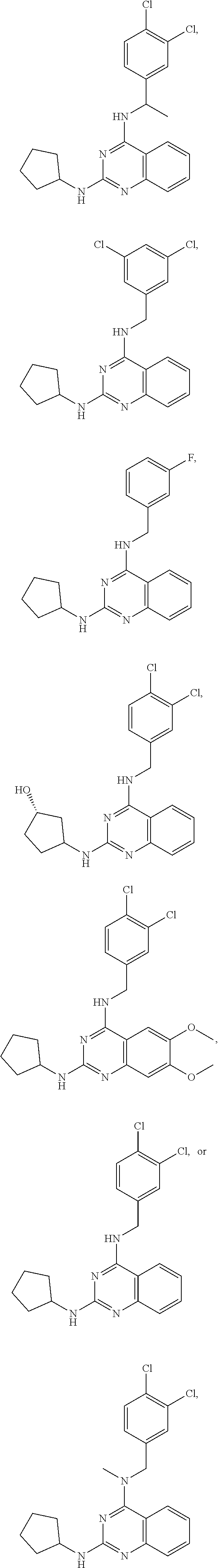

or a pharmaceutically acceptable salt thereof.

In one aspect, a compound can be present as:

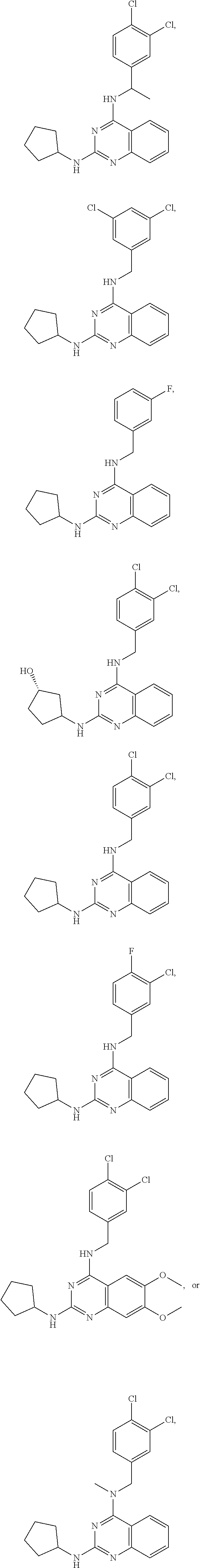

or a pharmaceutically acceptable salt thereof.

In one aspect, a compound can be present as:

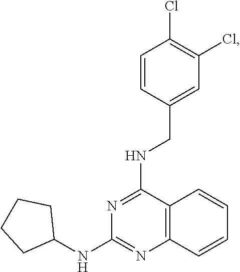 [SMILES: Clc1ccc(CNc2nc(NC3CCCC3)nc3ccccc23)cc1Cl]

or a pharmaceutically acceptable salt thereof.

It is contemplated that one or more compounds can optionally be omitted from the disclosed invention.

It is understood that the disclosed compounds can be used in connection with the disclosed methods, compositions, kits, and uses.

It is understood that pharmaceutical acceptable derivatives of the disclosed compounds can be used also in connection with the disclosed methods, compositions, kits, and uses. The pharmaceutical acceptable derivatives of the compounds can include any suitable derivative, such as pharmaceutically acceptable salts as discussed below, isomers, radiolabeled analogs, tautomers, and the like.

### C. PHARMACEUTICAL COMPOSITIONS

In one aspect, disclosed are pharmaceutical compositions comprising an effective amount of a disclosed compound and a pharmaceutically acceptable carrier.

Thus, in one aspect, disclosed are pharmaceutical compositions comprising an effective amount of a compound having a structure represented by a formula:

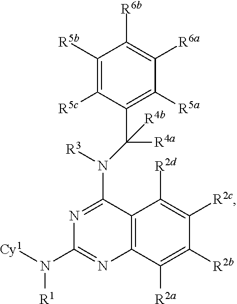

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{6a} and R^{6b} is independently selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least one of R^{6a} and R^{6b} is halogen, or a pharmaceutically acceptable salt thereof, and a pharmaceutically acceptable carrier.

In various aspects, the compound is a structure selected from:

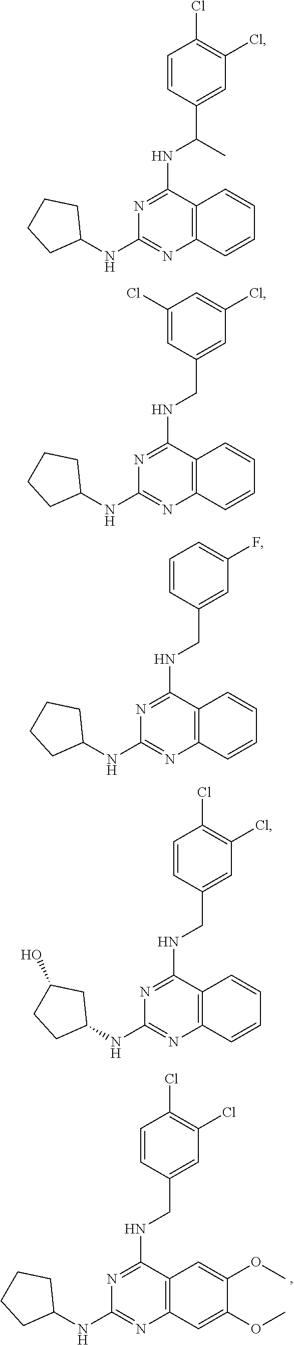

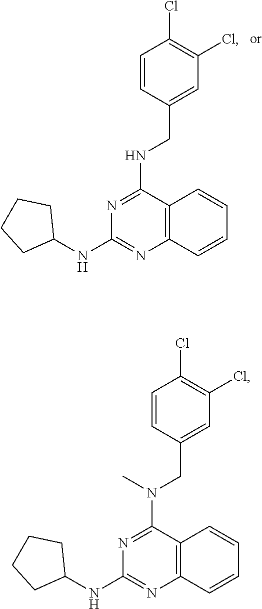 [SMILES: Clc1ccc(CNc2nc(NC3CCCC3)nc3ccccc23)cc1Cl ; CN(Cc1ccc(Cl)c(Cl)c1)c1nc(NC2CCCC2)nc2ccccc12]

or a pharmaceutically acceptable salt thereof.

In various aspects, the compound is:

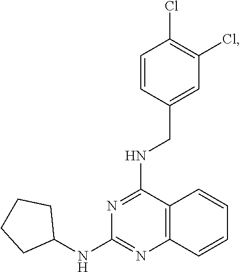 [SMILES: Clc1ccc(CNc2nc(NC3CCCC3)nc3ccccc23)cc1Cl]

or a pharmaceutically acceptable salt thereof.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, PA 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat a disorder associated with overexpression of an EYA protein. In a still further aspect the disorder is a vascular disease, a fibrosis-related disorder, hearing loss, or a metabolic disease.

In a further aspect, the pharmaceutical composition is used to treat a cancer that comprises a tumor that overexpress at least one EYA protein. In a still further aspect, the cancer is breast cancer, cervical cancer, ovarian cancer, liver cancer, or pancreatic cancer. In yet a further aspect, the cancer is triple-negative breast cancer (TNBC). In an even further aspect, the cancer is a pediatric cancer (e.g., leukemia).

It is understood that the disclosed compositions can be prepared from the disclosed compounds. It is also understood that the disclosed compositions can be employed in the disclosed methods of using.

### D. METHODS OF MAKING A COMPOUND

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Preferred methods include, but are not limited to, those described below. During any of the following synthetic sequences, it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; and T. W. Greene and P. G. M. Wuts, Protective Groups in Organic Chemistry, John Wiley & Sons, 1991, which are hereby incorporated by reference.

Reactions used to generate the compounds of this invention are prepared by employing reactions as shown in the following Reaction Schemes, as described and exemplified below. In certain specific examples, the disclosed compounds can be prepared by Route I, as described and exemplified below. The following examples are provided so that the invention might be more fully understood, are illustrative only, and should not be construed as limiting.

### 1. Route I

In one aspect, substituted quinazoline-2,4-diamines, or their pharmaceutically acceptable salts, can be prepared as shown below. Isolation and purification of the products is accomplished by standard procedures, which are known to a chemist of ordinary skill.

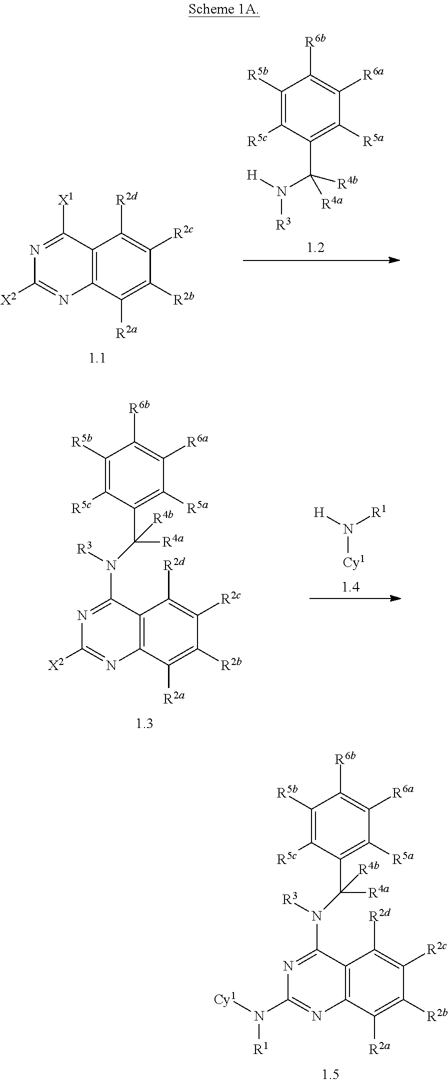

Compounds are represented in generic form, wherein X¹ and X² are independently halogen, and with substituents as noted in compound descriptions elsewhere herein. A more specific example is set forth below.

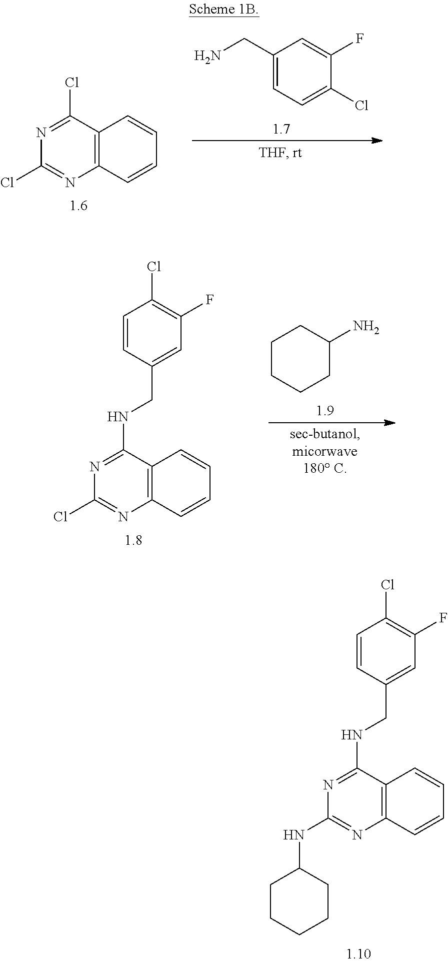 [SMILES: Clc1nc(Cl)c2ccccc2n1 ; NCc1ccc(Cl)c(F)c1 ; Fc1cc(CNc2nc(Cl)nc3ccccc23)ccc1Cl ; NC1CCCCC1 ; Fc1cc(CNc2nc(NC3CCCCC3)nc3ccccc23)ccc1Cl]

Referring to Scheme 1B above, condensation of an appropriate 2,4-dichloroquinazoline, e.g., 1.6 as shown above, with an appropriately substituted benzyl amine, e.g., 1.7 as shown above, in a suitable solvent such as THF, 2-methyl THF, DCE, or dioxane, at temperatures ranging from room temperature to 100° C., produces the desired mon-substituted aminoquinazoline, e.g., 1.8 as shown above. Appropriate quinazolines and appropriate benzyl amines are commercially available or prepared by methods known to one skilled in the art. Preferred conditions for this reaction include the use of THF as the solvent at room temperature. Treatment of an appropriate aminoquinazoline, e.g., 1.8 as shown above, with an appropriate cyclic amine, e.g., 1.9 as shown above, in a suitable alcoholic solvent such as sec-butanol, butanol, pentanol, ethanol, or t-butyl alcohol, under microwave irradiation conditions (120-180° C.), produces the desired quinazoline-2,4-diamine, e.g., 1.10 as shown above. Appropriate cyclic amines are commercially available or prepared by methods known to one skilled in the art. Preferred conditions for this reaction include the use of sec-butanol as the solvent under microwave irradiation conditions at a temperature of 180° C. As can be appreciated by one skilled in the art, the above reaction provides an example of a generalized approach wherein compounds similar in structure to the specific reactants above (compounds similar to compounds of type 1.1, 1.2, 1.3, and 1.4), can be substituted in the reaction to provide substituted substituted quinazoline-2,4-diamine derivatives similar to Formula 1.5.

Pharmaceutically acceptable salts of the disclosed compounds include the acid or base addition salts thereof. All salt formation reactions are typically carried out in solution. The resulting salt may precipitate out and be collected by filtration or may be recovered by evaporation of the solvent. The degree of ionization in the resulting salt may vary from completely ionized to almost non-ionized. Suitable non-toxic, acid-addition pharmaceutically acceptable salts include, but are not limited to, the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinofoate salts.

Suitable non-toxic, base-addition pharmaceutically acceptable salts include, but are not limited to, the aluminum, arginine, benzathine, calcium, choline, diethylamine, diolamine, glycine, lysine, magnesium, meglumine, olamine, potassium, sodium, tromethamine and zinc salts. For a review on suitable salts, see Handbook of Pharmaceutical Salts: Properties, Selection, and Use by Stahl and Wermuth (Wiley-VCH, 2002).

Also included within the scope of the present invention are all stereoisomers, geometric isomers, and tautomeric forms of the disclosed compounds, including compounds exhibiting more than one type of isomerism, and mixtures of one or more thereof.

The present invention also includes all pharmaceutically acceptable isotopically-labeled analogs of the disclosed compounds, in which one or more atoms are replaced by atoms having the same atomic number, but an atomic mass or mass number different from the atomic mass or mass number which predominates in nature.

### E. METHODS OF TREATING A DISORDER ASSOCIATED WITH OVEREXPRESSION OF AN EYA PROTEIN

In one aspect, disclosed are methods of treating a disorder associated with overexpression of an EYA protein in a subject in need thereof, the method comprising administering to the subject an effective amount of a disclosed compound, thereby treating the disorder. Examples of disorders for which the disclosed compounds, compositions, and methods can be useful include, but are not limited to, vascular diseases, fibrosis-related disorders, hearing loss, metabolic diseases, and cancer (e.g., a cancer that comprises a tumor that overexpresses at least one EYA protein).

Thus, in one aspect, disclosed are methods of treating a disorder associated with overexpression of an eyes absent (EYA) protein in a subject in need thereof, the method comprising administering to the subject an effective amount of a compound having a structure represented by a formula:

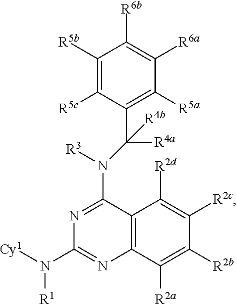

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{6a} and R^{6b} is independently selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least one of R^{6a} and R^{6b} is halogen, or a pharmaceutically acceptable salt thereof, wherein the disorder is a vascular disease, a fibrosis-related disorder, hearing loss, or a metabolic disease.

In various aspects, the disorder is associated with overexpression of eyes absent homolog 4 (EYA4) or eyes absent homolog 2 (EYA2). In a further aspect, the disorder is associated with overexpression of EYA4. In a still further aspect, the disorder is associated with overexpression of EYA2.

In various aspects, the compound modulates expression of one or more EYA proteins. In a further aspect, the compound modulates expression of EYA4 or EYA2. In a still further aspect, the compound modulates expression of EYA4. In yet a further aspect, the compound modulates expression of EYA2. In an even further aspect, the compound modulates expression of EYA4 and EYA2

In various aspects, the compound inhibits expression of one or more EYA proteins. In a further aspect, the compound inhibits expression of EYA2 or EYA4. In a still further aspect, the compound inhibits expression of EYA4. In yet a further aspect, the compound inhibits expression of EYA2. In an even further aspect, the compound inhibits expression of EYA2 and EYA4.

In various aspects, R^{6a} is halogen. In a still further aspect, either: (a) R^{6b} is chloro; (b) R^{6b} is hydrogen; or (c) R^{6b} is halogen and at least one of R^{5a}, R^{5b}, and R^{5c} is not hydrogen.

In various aspects, R¹ is hydrogen.

In various aspects, each of R^{2a}, R^{2b}, R^{2c}, R^{2d}, and R^{2e} is hydrogen.

In various aspects, R³ is hydrogen.

In various aspects, each of R^{4a} and R^{4b} is hydrogen.

In various aspects, each of R^{5a}, R^{5b}, and R^{5c} is hydrogen.

In various aspects, R^{6a} is chloro.

In various aspects, R^{6b} is halogen. In a further aspect, R^{6b} is chloro.

In various aspects, R^{6b} is hydrogen.

In various aspects, Cy¹ is an unsubstituted C3-C8 cycloalkyl. In a further aspect, Cy¹ is an unsubstituted C3-C6 cycloalkyl. In a still further aspect, Cy¹ is a cyclopentyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is an unsubstituted cyclopentyl.

In various aspects, the compound has a structure represented by a formula:

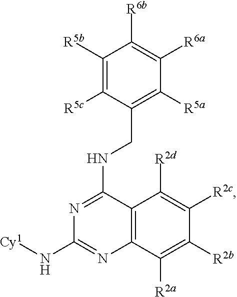

or a pharmaceutically acceptable salt thereof.

In various aspects, the compound has a structure represented by a formula:

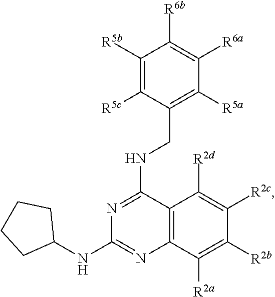

or a pharmaceutically acceptable salt thereof.

In various aspects, the compound has a structure represented by a formula:

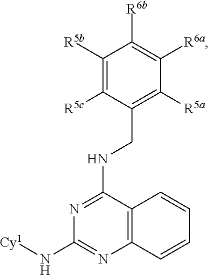

or a pharmaceutically acceptable salt thereof.

In various aspects, the compound has a structure represented by a formula:

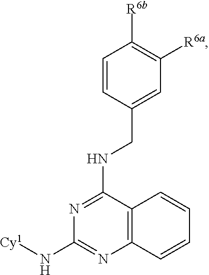

or a pharmaceutically acceptable salt thereof.

In various aspects, the compound has a structure represented by a formula:

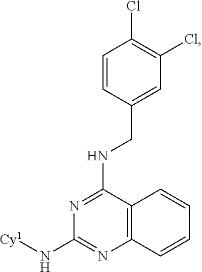

or a pharmaceutically acceptable salt thereof.

In various aspects, the compound is a structure selected from:

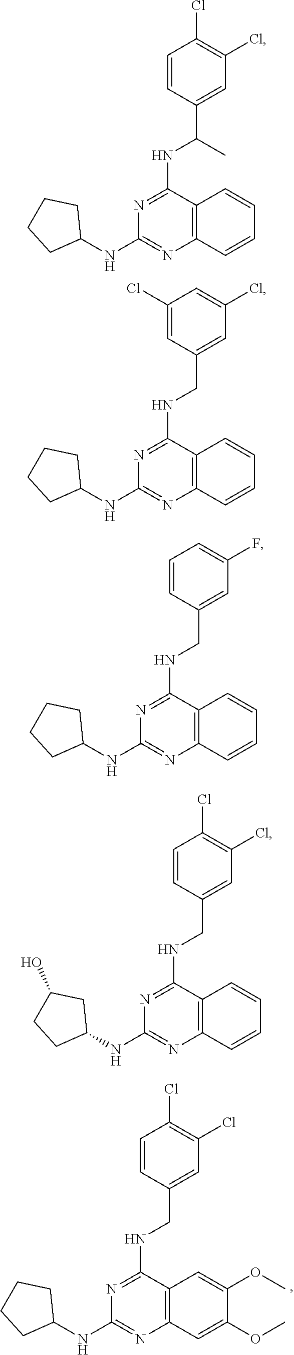

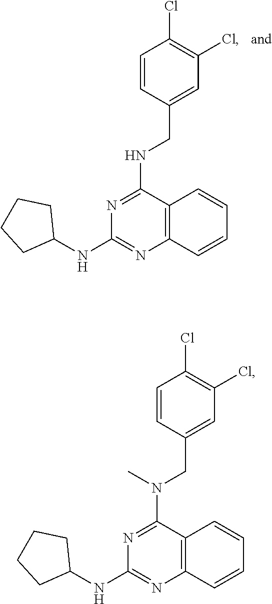 [SMILES: Clc1ccc(CNc2nc(NC3CCCC3)nc3ccccc23)cc1Cl ; CN(Cc1ccc(Cl)c(Cl)c1)c1nc(NC2CCCC2)nc2ccccc12]

or a pharmaceutically acceptable salt thereof.

In various aspects, the compound is:

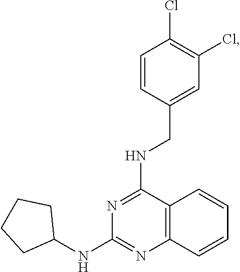 [SMILES: Clc1ccc(CNc2nc(NC3CCCC3)nc3ccccc23)cc1Cl]

or a pharmaceutically acceptable salt thereof.

In a further aspect, the subject has been diagnosed with a need for treatment of modification of the expression of one or more EYA proteins prior to the administering step. In a still further aspect, the subject has been diagnosed with a need for treatment of the disorder prior to the administering step.

In a further aspect, the subject is a mammal. In a still further aspect, the mammal is a human.

In a further aspect, the method further comprises the step of identifying a subject in need of treatment of the disorder.

In various aspects, the disorder is a vascular disease. Examples of vascular diseases include, but are not limited to dilated cardiomyopathy, cardiac hypertrophy, hypoxia-induced angiogenesis, atherosclerosis, peripheral artery disease, pericardial edema, carotid artery disease, a pulmonary embolism, collagen vascular disease, and cerebrovascular disease.

In various aspects, the disorder is a fibrosis-related disorder. Examples of fibrosis-related disorders include, but are not limited to, scleroderma, pulmonary fibrosis, rheumatoid arthritis, Crohn's disease, ulcerative colitis, myelofibrosis, systemic lupus erythematosus, idiopathic pulmonary fibrosis, non-alcoholic steatohepatitis (NASH), systemic sclerosis, and interstitial lung disease.

In various aspects, the disorder is hearing loss.

In various aspects, the disorder is a metabolic disease. Examples of metabolic diseases include, but are not limited to, familial hypercholesterolemia, Gaucher disease, Hunter syndrome, Krabbe disease, Maple syrip urine disease, metachromatic leukodystrophy, mitochondrial encephalopathy, lactic acidosis, stroke-like episodes (MELAS), Niemann-Pick, phenylketonuria (PKU), porphyria, Tay-Sachs disease, and Wilson's disease. In various further aspects, the metabolic disease is a fatty liver disease or a neoplasia (e.g., neoplasia of the heart or the liver).

In a further aspect, the effective amount is a therapeutically effective amount. In a still further aspect, the effective amount is a prophylactically effective amount.

In a further aspect, the method further comprises administering to the subject an agent known to treat a vascular disease. Examples of agents known to treat a vascular disease include, but are not limited to, antiplatelet medicines (e.g., aspirin, clopidogrel), statins, angiotensin II receptor blockers (ARBs), and ACE inhibitors.

In a further aspect, the method further comprises administering to the subject an anti-fibrotic agent. Examples of anti-fibrotic agents include, but are not limited to, nintedanib, pirfenidone, corticosteroids, azathioprine, chloroquines, and ruxolitinib.

In a further aspect, the method further comprises administering to the subject an agent known to treat hearing loss. Examples of agents known to treat hearing loss include, but are not limited to, aminoglycoside antibiotics, platinum-containing chemotherapy agents, loop diuretics (e.g., furosemide), and nonsteroidal anti-inflammatory agents.

In a further aspect, the method further comprises administering to the subject an agent known to treat a metabolic disease. Examples of agents known to treat a metabolic disease include, but are not limited to, ACE inhibitors, angiotensin receptor blockers, diuretics, beta blockers, statins, niacin, omega fatty acids, and insulin sensitizers (e.g., thiazolidinediones).

In a further aspect, the compound and the agent are administered sequentially. In a still further aspect, the compound and the agent are administered simultaneously.

In a further aspect, the compound and the agent are co-formulated. In a still further aspect, the compound and the agent are co-packaged.

In a further aspect, the compound is administered as a single active agent.

### F. METHODS OF TREATING A CANCER THAT COMPRISES A TUMOR THAT OVEREXPRESSES AT LEAST ONE EYA PROTEIN

In one aspect, disclosed are methods of treating cancer in a subject in need thereof, the method comprising administering to the subject an effective amount of a disclosed compound, wherein the cancer comprises a tumor that overexpresses at least one eyes absent (EYA) protein.

Thus, in one aspect, disclosed are methods of treating cancer in a subject in need thereof, the method comprising administering to the subject an effective amount of a compound having a structure represented by a formula:

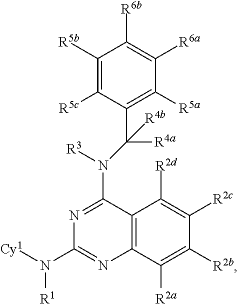

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{6a} and R^{6b} is independently selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least one of R^{6a} and R^{6b} is halogen, or a pharmaceutically acceptable salt thereof, wherein the cancer comprises a tumor that overexpresses at least one eyes absent (EYA) protein.

In various aspects, the tumor overexpresses EYA4. In a further aspect, the tumor overexpresses EYA2.

In various aspects, R^{6a} is halogen. In a still further aspect, either: (a) R^{6b} is chloro; (b) R^{6b} is hydrogen; or (c) R^{6b} is halogen and at least one of R^{5a}, R^{5b}, and R^{5c} is not hydrogen.

In various aspects, R¹ is hydrogen.

In various aspects, each of R^{2a}, R^{2b}, R^{2c}, R^{2d}, and R^{2e} is hydrogen.

In various aspects, R³ is hydrogen.

In various aspects, each of R^{4a} and R^{4b} is hydrogen.

In various aspects, each of R^{5a}, R^{5b}, and R^{5c} is hydrogen.

In various aspects, R^{6a} is chloro.

In various aspects, R^{6b} is halogen. In a further aspect, R^{6b} is chloro.

In various aspects, R^{6b} is hydrogen.

In various aspects, Cy¹ is an unsubstituted C3-C8 cycloalkyl. In a further aspect, Cy¹ is an unsubstituted C3-C6 cycloalkyl. In a still further aspect, Cy¹ is a cyclopentyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is an unsubstituted cyclopentyl.

In various aspects, the compound has a structure represented by a formula:

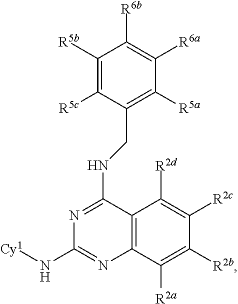

or a pharmaceutically acceptable salt thereof.

In various aspects, the compound has a structure represented by a formula:

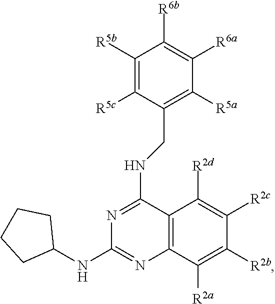

or a pharmaceutically acceptable salt thereof.

In various aspects, the compound has a structure represented by a formula:

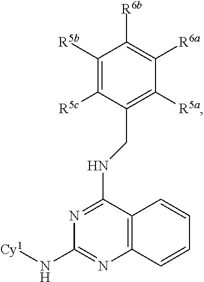

or a pharmaceutically acceptable salt thereof.

In various aspects, the compound has a structure represented by a formula:

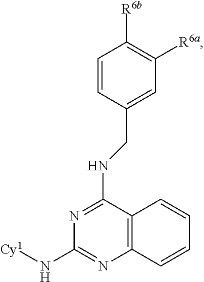

or a pharmaceutically acceptable salt thereof.

In various aspects, the compound has a structure represented by a formula:

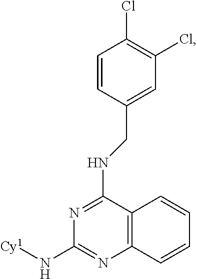

or a pharmaceutically acceptable salt thereof.

In various aspects, the compound is a structure selected from:

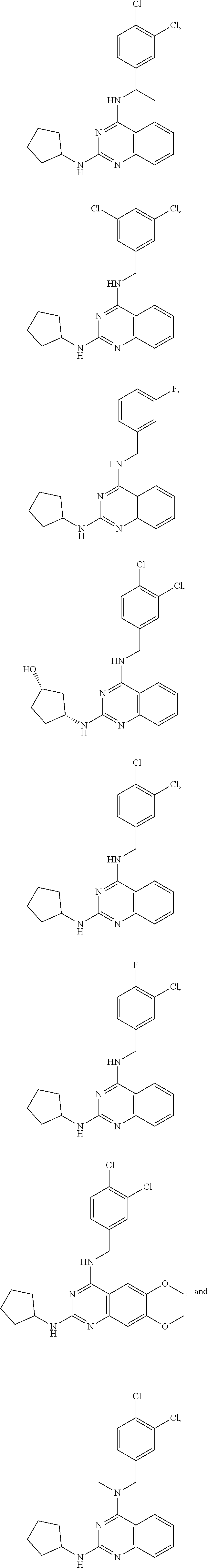

or a pharmaceutically acceptable salt thereof.

In various aspects, the compound is:

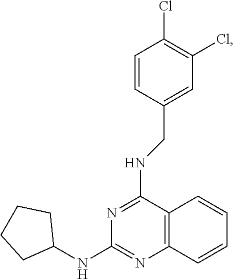 [SMILES: Clc1ccc(CNc2nc(NC3CCCC3)nc3ccccc23)cc1Cl]

or a pharmaceutically acceptable salt thereof.

In a further aspect, the subject has been diagnosed with a need for treatment of cancer prior to the administering step. In a still further aspect, the subject is at risk for developing cancer prior to the administering step.

In a further aspect, the subject is a mammal. In a still further aspect, the mammal is a human.

In a further aspect, the method further comprises the step of identifying a subject in need of treatment of cancer.

In various aspects, the cancer is selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma). In a further aspect, the cancer is breast cancer, cervical cancer, ovarian cancer, liver cancer, or pancreatic cancer. In a still further aspect, the cancer is triple-negative breast cancer (TNBC).

In various aspects, treating cancer comprises limiting metasis and overcoming chemotherapy resistance caused by endoreplication and genomic rearrangements. In a further aspect, the endoreplication is in response to stress. In a further aspect, the endoreplication is a result of missed mitosis initiation.

In various aspects, the cancer is a pediatric cancer. In a further aspect, the pediatric cancer is Ewing sarcoma, osteosarcoma, leukemia, neuroblastoma, retinoblastoma, or melanoma. In a still further aspect, the pediatric cancer is leukemia.

In a further aspect, the effective amount is a therapeutically effective amount. In a still further aspect, the effective amount is a prophylactically effective amount.

In a further aspect, the method further comprises administering a chemotherapeutic agent to the subject. Examples of chemotherapeutic agents include, but are not limited to, alkylating agents (e.g., carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, streptozocin), antimetabolite agents (e.g., gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, thioguanine), antineoplastic antibiotic agents (e.g., doxorubicin, mitoxantrone, bleomycin, daunorubicin, dactinomycin, epirubicin, idarubicin, plicamycin, mitomycin, pentostatin, valrubicin), mitotic inhibitor agents (e.g., irinotecan, topotecan, rubitecan, cabazitaxel, docetaxel, paclitaxel, etopside, vincristine, ixabepilone, vinorelbine, vinblastine, teniposide), and mTor inhibitor agents (e.g. everolimus, siroliumus, temsirolimus).

In a further aspect, the compound and the agent are administered sequentially. In a still further aspect, the compound and the agent are administered simultaneously.

In a further aspect, the compound and the agent are co-formulated. In a still further aspect, the compound and the agent are co-packaged.

In a further aspect, the compound is administered as a single active agent.

### G. ADDITIONAL METHODS OF USING THE COMPOUNDS

The compounds and pharmaceutical compositions of the invention are useful in treating or controlling disorders associated with overexpression of an eyes absent (EYA) protein in its wild type or mutated version (e.g., a vascular disease, a fibrosis-related disorder, hearing loss, a metabolic disease). The compounds and pharmaceutical compositions of the invention are also useful in treating or controlling cancer that comprises a tumor that overexpresses at least one eyes absent (EYA) protein.

To treat or control the condition, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a disorder associated with overexpression of an eyes absent (EYA) protein (e.g., a vascular disease, a fibrosis-related disorder, hearing loss, a metabolic disease) or with a need for treatment of a cancer that comprises a tumor that overexpresses at least one eyes absent (EYA) protein.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a disorder associated with overexpression of an eyes absent (EYA) protein (e.g., a vascular disease, a fibrosis-related disorder, hearing loss, a metabolic disease) or for prevention of a cancer that comprises a tumor that overexpresses at least one eyes absent (EYA) protein.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

### 2. Use of Compounds

In one aspect, the invention relates to the use of a disclosed compound or a product of a disclosed method. In a further aspect, a use relates to the manufacture of a medicament for the treatment of a disorder associated with overexpression of an eyes absent (EYA) protein (e.g., a vascular disease, a fibrosis-related disorder, hearing loss, a metabolic disease). In a still further aspect, a use relates to the manufacture of a medicament for the treatment of a cancer that comprises a tumor that overexpresses at least one eyes absent (EYA) protein.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of a disorder associated with overexpression of an eyes absent (EYA) protein in a subject. In one aspect, the use is characterized in that the subject is a human. In one aspect, the use is characterized in that the disorder associated with overexpression of an eyes absent (EYA) protein is a vascular disease, a fibrosis-related disorder, hearing loss, or a metabolic disease.

In various aspects, the use relates to a treatment of disorder associated with overexpression of an eyes absent (EYA) protein wherein the disorder is a vascular disease, a fibrosis-related disorder, hearing loss, or a metabolic disease

In various aspects, the use relates to a treatment of cancer comprising a tumor having an overexpression of an eyes absent (EYA) protein.

In a further aspect, the use relates to the manufacture of a medicament for the treatment of a disorder associated with overexpression of an eyes absent (EYA) protein in a subject.

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of a disorder associated with overexpression of an eyes absent (EYA) protein in a mammal. In a further aspect, the invention relates to the use of a disclosed compound or a disclosed product in the manufacture of a medicament for the treatment of a cancer that comprises a tumor that overexpresses at least one eyes absent (EYA) protein. In a still further aspect, the cancer is breast cancer (e.g., triple-negative breast cancer), cervical cancer, ovarian cancer, liver cancer, pancreatic cancer, or a pediatric cancer (e.g., leukemia).

### 3. Manufacture of a Medicament

In one aspect, the invention relates to the manufacture of a medicament for use in treating a disorder associated with overexpression of an eyes absent (EYA) protein such as, for example, a vascular disease, a fibrosis-related disorder, hearing loss, or a metabolic disease, in a subject having the condition, the method comprising combining a therapeutically effective amount of a disclosed compound or product of a disclosed method with a pharmaceutically acceptable carrier or diluent. In another aspect, the invention relates to the manufacture of a medicament for use in treating a cancer comprising a tumor that overexpresses at least one eyes absent (EYA) protein (e.g., breast cancer, triple-negative breast cancer (TNBC), cervical cancer, ovarian cancer, liver cancer, pancreatic cancer, pediatric cancer or leukemia), in a subject having the condition, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the treatment of a disorder associated with overexpression of eyes absent protein (e.g., a vascular disease, a fibrosis-related disorder, hearing loss, or a metabolic disease) or in the treatment of a cancer that comprises a tumor that overexpresss at least one eyes absent (EYA) protein. The dose administered to an animal, particularly a human, in the context of the present invention should be sufficient to affect a therapeutic response in the animal over a reasonable timeframe. One skilled in the art will recognize that dosage will depend upon a variety of factors including the condition of the animal and the body weight of the animal.

The total amount of the compound of the present disclosure administered in a typical treatment is preferably between about 0.05 mg/kg and about 100 mg/kg of body weight for mice, and more preferably between 0.05 mg/kg and about 50 mg/kg of body weight for mice, and between about 100 mg/kg and about 500 mg/kg of body weight for humans, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing, and frequency of administration as well as the existence, nature and extent of any adverse side effects that might accompany the administration of the compound and the desired physiological effect. It will be appreciated by one of skill in the art that various conditions or disease states, in particular chronic conditions, or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

### H. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Chemistry Experimentals

All reactions were carried out in an oven-dried glassware under argon atmosphere using standard gas-tight syringe, cannula, and septa. The reaction temperatures were measured externally. Stirring was achieved with oven dried magnetic bars. All the reactions were done in anhydrous solvents (CH₂Cl₂, THF, MeOH) purchased from Sigma-Aldrich. All commercially purchased reagents were used without purification. The reactions were monitored by thin-layer chromatography (TLC) on a pre-coated silica gel (60 F254) glass plates from EMD Millipore and visualized using UV light (254 nm). Purification of the compounds was performed on Teledyne-ISCO Combiflash Rf 200 purification system using Redisep Rf® normal phase silica gel columns 230-400 mesh. ESI-MS spectra were recorded on a BioTof-2 time-of-flight mass spectrometer. Proton NMR spectra were recorded on a Varian Unity 400 NMR spectrometer operating at 400 MHz calibrated to the solvent peak and TMS peak. The chemical formula and Exact Mass for target compounds were determined from the (M+H)⁺ by high resolution mass spectroscopy using an Agilent 6210 Electrospray Time of Flight.

### A. General Experimental

All operations were carried out at room or ambient temperature, that is, in the range of 18-25° C.; evaporation of solvent was carried out using a rotary evaporator under reduced pressure with a bath of up to 50° C.; reactions were monitored by thin layer chromatography (tlc) and reaction times are given for illustration only. Unless otherwise indicated all reactions were conducted in standard commercially available glassware using standard synthetic chemistry methods and setup. All air- and moisture-sensitive reactions were performed under nitrogen atmosphere with dried solvents and glassware under anhydrous conditions. Starting materials and reagents were commercial compounds of the highest purity available and were used without purification (See list of specific reagents below). Solvents used for reactions were indicated as of commercial dry or extra-dry or analytical grade. Analytical thin layer chromatography was performed on aluminum plates coated with Merck Kieselgel 60F254 and visualized by UV irradiation (254 nm) or by staining with a solution of potassium permanganate. Flash column chromatography was performed on Biotage Isolera One 2.2 using commercial columns that were pre-packed with Merck Kieselgel 60 (230-400 mesh) silica gel. Final compounds for biological testing are all ≥95% purity as determined by HPLC-MS and ¹H NMR. ¹H NMR experiments were recorded on Agilent DD2 400 MHz spectrometers at ambient temperature. Samples were dissolved and prepared in deuterated solvents (CDCl₃, CD₃OD and DMSOd₆) with residual solvents being used as the internal standard in all cases. All deuterated solvent peaks were corrected to the standard chemical shifts (CDCl₃, dH=7.26 ppm; CD₃OD, dH=3.31 ppm; DMSOd₆, dH=2.50 ppm). Spectra were all manually integrated after automatic baseline correction. Chemical shifts (d) are given in parts per million (ppm), and coupling constants (J) are given in Hertz (Hz). The proton spectra are reported as follows: d (multiplicity, coupling constant J, number of protons). The following abbreviations were used to explain the multiplicities: app=apparent, b=broad, d=doublet, dd=doublet of doublets, ddd=doublet of doublet of doublets, dddd=doublet of doublet of doublet of doublets, m=multiplet, s=singlet, t=triplet. All samples were analyzed on Agilent 1290 series HPLC system comprised of binary pumps, degasser and UV detector, equipped with an auto-sampler that is coupled with Agilent 6150 mass spectrometer. Purity was determined via UV detection with a bandwidth of 170 nm in the range from 230-400 nm. The general LC parameters were as follows: Column—Zorbax Eclipse Plus C18, size 2.1×50 mm; Solvent A: 0.10% formic acid in water, Solvent B: 0.00% formic acid in acetonitrile; Flow rate—0.7 mL/min; Gradient: 5% B to 95% B in 5 min and hold at 95% B for 2 min; UV detector—channel 1=254 nm, channel 2=254 nm. Mass detector Agilent Jet Stream—Electron Ionization (AJS-ES).

The following abbreviations are used:
THF: tetrahydrofuran
DCM or CH₂Cl₂: dichloromethane
DCE: dichloroethane
NaHCO₃: sodium bicarbonate
HCl: hydrogen chloride
MgSO₄: magnesium sulfate
Na₂SO₄: sodium sulfate
DME: dimethoxyethane
n-BuLi: n-butyllithium
DMF: dimethylformamide
DMSO: dimethylsulfoxide
Et₂O: diethyl ether
MeOH: methanol
EtOAc: ethyl acetate

### a. Representative Synthesis of N²-cyclopentyl-N⁴-(3,4-dichlorobenzyl)quinazoline-2,4-diamine (AT-301; No. 38)

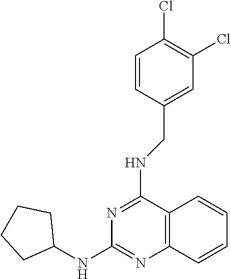 [SMILES: Clc1ccc(CNc2nc(NC3CCCC3)nc3ccccc23)cc1Cl]

2-chloro-N-(3,4-dichlorobenzyl)quinazolin-4-amine (3): To a stirring solution of 2,4-dichloroquinazoline (5.0 g, 25.1 mmol) in THF (125 mL) at room temperature was added 3,4-dichlorobenzylamine (4.0 mL, 30.3 mmol). The mixture was stirred for 24 hr at room temperature, during which time a precipitate formed. The slurry was filtered and washed with hexanes. The collected filtrate was slurried with DCM/hexanes and filtered. The filtrate was washed with hexanes, collected and dried under reduce vacuum to yield 4.4 g (52% yield) of 2-chloro-N-(3,4-dichlorobenzyl)quinazolin-4-amine (3) as a white powder.

N²-cyclopentyl-N⁴-(3,4-dichlorobenzyl)quinazoline-2,4-diamine (AT-301; 38): To a 30 mL microwave reaction vial was added 2-chloro-N-(3,4-dichlorobenzyl)quinazolin-4-amine 3 (1.0 g, 2.95 mmol), sec-butanol (15 mL) and cyclopentylamine (251 mg, 2.95 mmol). The mixture was irradiated in an Anton Par microwave reactor for 30 minutes at 180° C. The reaction was cooled to room temperature, during which time a precipitate formed. The solids were filtered, washed with hexanes and dried under reduced pressure to yield N²-cyclopentyl-N⁴-(3,4-dichlorobenzyl)quinazoline-2,4-diamine (970 mg, 85% yield) as a white powder.

### b. Additional Exemplary Compounds

Compounds listed in Table 1 below were prepared as detailed above for Compound 4.

TABLE 1

| No. | Structure | MW |
|---|---|---|
| 1 | 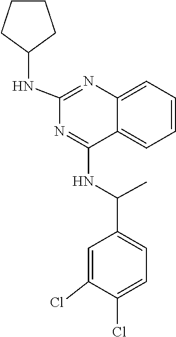 [SMILES: CC(Nc1nc(NC2CCCC2)nc2ccccc12)c1ccc(Cl)c(Cl)c1] | 401.3 |
| 2 | 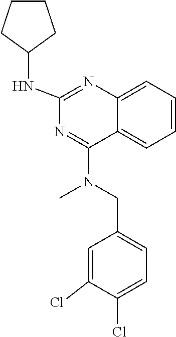 [SMILES: CN(Cc1ccc(Cl)c(Cl)c1)c1nc(NC2CCCC2)nc2ccccc12] | 401.3 |
| 3 | 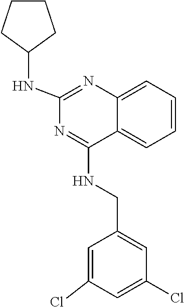 [SMILES: Clc1cc(Cl)cc(CNc2nc(NC3CCCC3)nc3ccccc23)c1] | 387.3 |
| 4 | 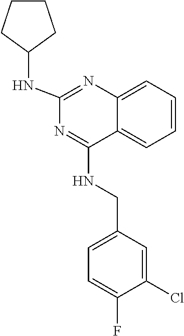 [SMILES: Fc1ccc(CNc2nc(NC3CCCC3)nc3ccccc23)cc1Cl] | 370.9 |
| 5 | 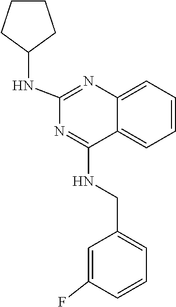 [SMILES: Fc1cccc(CNc2nc(NC3CCCC3)nc3ccccc23)c1] | 336.4 |
| 6 | 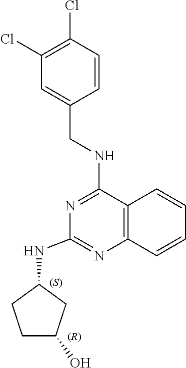 | 403.3 |
| 7 | 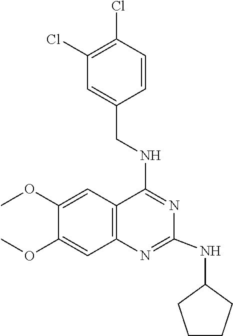 [SMILES: COc1cc2nc(NC3CCCC3)nc(NCc3ccc(Cl)c(Cl)c3)c2cc1OC] | 447.4 |
| 8 | 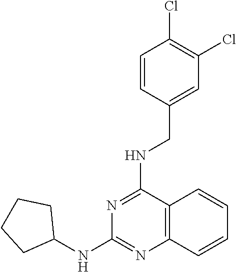 [SMILES: Clc1ccc(CNc2nc(NC3CCCC3)nc3ccccc23)cc1Cl] | 387.3 |
| 9 | 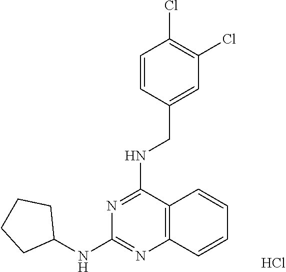 [SMILES: Clc1ccc(CNc2nc(NC3CCCC3)nc3ccccc23)cc1Cl ; Cl] | 387.3 |

### 2. RAD51 Dephosphorylation by the EYA4 Tyrosine Phosphatase Controls its Filament Formation and its Activity in Homologous Recombination

The observation that EYA4 is hypermethylated and possibly overexpressed in triple-negative breast cancer samples prompted the study of its cellular role beyond organogenesis. EYA4 was inactivated or overexpressed in a variety of cell lines and the resulting phenotypes were investigated. In addition, the DNA repair efficiency was evaluated and the identity of the EYA4 targets and other interacting partners were sought.

Novel ssDNA-binding activity of EYA4 was uncovered, which greatly stimulated its phosphatase activity. In addition to binding ssDNA, EYA4 is chromatin bound through direct interaction with histones. It was found that upon DNA damage, EYA4 dephosphorylates H2AX on residue pY142, as previously suggested by homology. Further it was found that EYA4 is also phosphorylated in response to irradiation, which causes it to detach from the chromatin, likely to facilitate access to DSBs by the DNA repair machinery. In addition, it was found that EYA4 targets RAD51 on residue Y315, a tyrosine which influences RAD51 polymerization and presynaptic filament formation. In addition, it was found that while cells depleted for EYA4 are sensitive to genotoxic stress, deficient for HR, and exhibit chromatin compaction defects, overexpression of EYA4 drives the accumulation of hyper-active and stable dephosphorylated RAD51 protein, which forms longer presynaptic filaments in vitro. Conversely it was found that phosphorylation of RAD51 reduces its DNA binding activity. Taken together, these findings indicate that RAD51 phosphorylation status changes the nature of the filament and controls the recombination function of RAD51. Thus, it was determined that EYA4 is a key player of DDR. Therefore, overexpression of EYA4 in tumors could yield accumulation of RAD51, which is linked with hyperrecombination and drug resistance phenotypes, even in the absence of copy number variation. These discoveries suggest that the EYA proteins, and, in particular, the EYA4 protein is a druggable target that could be used in the treatments of disorders associated with overexpression of EYA proteins including, but not limited to cancer treatments to limit metastasis and combat drug resistance caused by elevated RAD51 levels or secondary mutations in HR genes that restore HR (H. L. Klein (2008) DNA Repair (Amst) 7, 686-693; Y. Feng et al. (2021) Cancer Cell Int 21, 249).

### A. Materials and Methods

### i. Cell Culture and Maintenance, Transfections, and Stable Cell Lines Establishment

Cell lines were obtained from ATCC, U2OS-DiVA gifted by Gaelle Legube, and U2OS/DR-GFP gifted by Jeremy Stark were maintained in cell-adhesion treated vessels at 37° C. in 5% CO₂ incubators. Cells were cultured in Dulbecco's Modified Eagle Medium (DMEM) (Gibco®) supplemented with 10% fetal bovine serum (FBS) and passaged at 80% confluence or less. DiVA were supplemented with Glutamax (Gibco). For virus production 1.2*106 HEK 293FT cells were reverse-transfected using Lipofectamine® 2000 Reagent (Invitrogen™) with MISSION® TRC2 pLKO.5-Puro (Sigma-Aldrich®) empty vector or MISSION® TRC2 pLKO.5-Puro (Sigma-Aldrich®) EYA4 shRNA constructs (shRNA1, TRCN0000244430; shRNA2, TRCN0000218273; shRNA3, TRCN0000244429) and Lenti-vpak plasmids from OriGene to create lentivirus particles. Viruses were harvested at 48 and 72 hours post transfection and filtered through a 0.45 μm filter, then used to infect cell lines with polybrene (4 μg/mL) in a 6 cm dish. Stable cell lines were selected after 48 hours using 1-2 μg/mL of puromycin. For complementation, stable cells expressing shRNA1 were transfected with the pcDNA™3.1/nV5-DEST construct coding for EYA4-resistant (full length, FL) and selected with 500 μg/mL geneticin. For DSB induction, DiVA cells were treated for 4 h with 300 nM hydroxytamoxifen (40HT) added directly to the culture medium. The origin of all cells was confirmed by short tandem repeat (STR) analysis. All cells were regularly tested for Mycoplasma and cell of origin was validated by STR profiling.

### ii. RT-qPCR and Western Blot Analyses

Total RNA was isolated by phenol chloroform extraction (TRIzol, Invitrogen) followed by nucleic acid precipitation. The GoScript Reverse Transcription System (Promega) was used to generate first-strand cDNA. Real-Time quantitative PCR (RT-qPCR) was performed using TaqMan probes for human EYA4 (Invitrogen, Hs01012406_mH) and human 18S (Invitrogen, Hs99999901_s1) to amplify 70 bp and 187 bp fragments, respectively. The relative expression of EYA4 was determined using 2^{-ΔΔCt} method with 18S as an endogenous control for normalization. Western blot analysis was conducted according to our standard procedures (C. Wiese et al., Mol Cell 28, 482-490 (2007)). Briefly, cells were lysed on ice in RIPA buffer (10 mM Tris-HCl (pH 8.0), 1 mM EDTA, 0.5 mM EGTA, 1% Triton X-100, 0.1% sodium deoxycholate, 0.1% SDS, 140 mM NaCl) supplemented with cOmplete Mini EDTA-free protease inhibitor cocktail (Roche), 1 mM PMSF, 1 mM Na3VO4, 1 mM NaF, 1 mM benzamidine and 0.025 U/μL benzonase, sonicated 2 minutes (40%) in an ultrasonic water bath. The primary antibodies were: EYA4 (Abcam), γH2AX (S139) (Millipore), β-Tubulin (9F3, CST) and β-Actin (C-4, Santa Cruz).

### iii. Indirect Immunofluorescence

Indirect immunofluorescence was performed as described elsewhere (B. de la Pe5a Avalos, E. Dray. Journal of Visualized Experiments 160, e61447 (2020)). Stable cell lines expressing control or shRNA plasmid were grown on coverslips for 24 hrs and treated with 4 Gy γ-rays. Cell nuclei were pre-extracted with nuclear extraction buffer (NEB; 10 mM PIPES (pH 6.8), 100 mM NaCl, 300 mM sucrose, 3 mM MgCl₂, 1 mM EGTA (pH 8.0), 0.5% Triton X-100) for 2 minutes at RT and fixed with 4% paraformaldehyde (PFA) for 10 minutes at 4° C. Nuclei were blocked in PBS with 5% BSA and 0.3% Triton X-100 for 2 hrs at RT, immunoblotted with a primary antibody (1:500 dilution in PBS with 1% BSA and 0.3% Triton X-100) for 2 hours at RT, followed by secondary antibody (2 μg/mL in PBS with 1% BSA and 0.3% Triton X-100) for 2 hours at RT. DNA was counterstained with DAPI. Slides were viewed on an Olympus FV3000 confocal microscope. Primary antibodies were: γH2AX (S139) (Millipore), pH2AX (Y142) (Abcam), EYA4 (Abcam), pRPA (S4/S8) (Bethyl), pRPA (S33) (Bethyl), RAD51 (H-92, Santa Cruz) and α-pRAD51 (Y315) (Abcam). Secondary antibodies were: α-Mouse (Abcam, Alexa Fluor 647), α-Rabbit (Abcam, Alexa Fluor 488), α-Mouse (Abcam ab, Alexa Fluor 488) and α-Rabbit (Santa Cruz, CFL-647). The number of nuclear foci and their colocalization was quantified using CellProfiler. When indicated, cells were irradiated (4 and 10 Gy) in regular growth medium and a Gammacell® 40 Exactor (radiation source: caesium¹³⁷) unit.

### iv. Proteins Expression and Purification

EYA4 full-length, cloned in pEGFP-C1 was expressed in mammalian cells using the expi293 expression system (ThermoFisher). 250 mL of culture was infected then grown for four days, and cell pellets were resuspended in lysis buffer (50 mM Tris-HCl (pH 8.9), 150 mM NaCl, 10% sucrose, 10% glycerol, 0.5 mM EDTA, 1 mM TCEP, 1 mM PMSF, 0.5% Igepal and protease inhibitors), sonicated 20 times for 15 seconds (50%). and the lysate was clarified by centrifugation (20,000×g, 30 minutes). The supernatant was diluted in 50 mM Tris-HCl (pH 8.9), 10% glycerol, and loaded onto a 7.4 mL Source 30 Q column equilibrated in buffer A (lysis+75 mM NaCl). The protein was fractionated in 4 mL fractions using a linear gradient to 100% of buffer B (buffer A+1 M NaCl). Fractions containing the peak EYA4 were pooled and incubated with 1 mL of agarose resin anti-GFP for 2 hrs at 4° C. The resin was collected, washed with 20 CV of buffer B, followed by 10 CV of buffer C (50 mM Tris-HCl (pH 8), 150 mM NaCl, 10% glycerol). GFP-EYA4 was either left on beads for interaction studies or eluted by cleavage of the GFP-tag with TEV protease (4° C. 2 h). EYA4 was collected the next day by flow in buffer C. TEV was removed by incubating elution on Ni-NTA2+ resin for 1 hr. Flow through and washes (buffer C) were then pooled, buffer exchanged in storage buffer (50 mM Tris-HCl (pH 8.0), 150 mM NaCl, 30% glycerol) and concentrated before storage.

### v. Phosphatase Assays

To measure phosphatase activity, increasing concentrations of EYA4 (0-200 nM, alone or pre-incubated with ssDNA) were incubated in the presence of 400 nM potential substrates (peptides sourced from Genscript, see list and sequence in figures) in 50 μL of reaction buffer A (50 μM MES pH 6, 2 mM MgCl₂, 50 μM DTT) for 1 h at 37° C. At the end of the first incubation, the reaction mix containing malachite green was added to phosphatase or ATPase reactions, and the mixture was further incubated for 30 minutes at 37° C. Plates were read at 620 nm in a plate reader. The quantity of phosphatase released was inferred from the standard curve prepared as per manufacturer recommendation (Sigma).

### vi. Electrophoretic Mobility Shift Assay (EMSA)

Electrophoretic Mobility Shift Assay was performed with primers (short system, IDT, see DNA substrates (below)) or Phi-X DNA (long system, sourced from NEB) as previously described (C. Wiese et al., Mol Cell 28, 482-490 (2007), M. H. Dunlop et al., J Biol Chem 286, 37328-37334 (2011)). Increasing concentrations of purified protein was incubated with fixed amounts of DNA in 10 μL of reaction buffer (50 mM Tris-HCl (pH 7.5), 150 mM NaCl, 1 mM MgCl₂, 1 mM dithiothreitol, 100 μg/mL BSA and 2.5 mM ATP) at 37° C. for 20 min. The reaction mixtures were resolved in 8% polyacrylamide gels in TBE buffer (100 mM Tris-borate (pH 8.3), 2 mM EDTA), for the short system, followed by imaging at 488 or 647 nm wavelength and quantification using QuantumStudio (BioRad). The long system EMSA were resolved on a 1% Agarose gel, stained with ethidium bromide, destained in water, and imaged under UV excitation on a BioRad imager then quantified.

### vii. Microscale Thermophoresis (MST)

A total of 12 concentrations of ligand proteins (e.g., EYA (1-365)) were serially diluted by 1:1 using MST buffer from 5 uM to 2.4 nM, 10 ul of each dose of ligand protein was mixed with 10 ul of cy5-labeled dsDNA/ssDNA supernatant. The mixture of EYA and DNA was loaded into premium capillaries for MST assay with parameters set up at auto-detected excitation power and medium MST power. Binding affinity was analyzed by MO. Affinity Analysis software (version 2.1.3, NanoTemper Technologies) using the signal from an MST-on time of 20 s. Three individual experiments were merged to generate standard deviation.

### viii. Electron Microscopy

The reaction was carried out at 37° C. in buffer containing 2 mM ATP, 2.5 mM MgCl₂, 50 mM KCl, and no BSA. To assemble nucleoprotein filaments, RAD51 (2.4 μM) was incubated with an 83mer Oligonucleotide polydT (7.2 μM nucleotides) for 5 min and then the reaction mixtures were diluted 8-fold with the same buffer, and a 4 μl aliquot was applied to 400-mesh grids coated with carbon film and which had previously been glow-discharged in air. After staining for 30 s with 4% uranyl acetate, the samples were examined in a JEOL JSM-6610LV electron microscope equipped with a tungsten filament at 100 keV. Digital images were captured with a charge-coupled device camera at a nominal magnification of ×63,000.

### ix. Immunoprecipitation (IP) and Mass Spectrometry

1.2×106 HeLa cells were reverse transfected with 1 μg of DNA (empty vector or pEGFPC1-EYA4). Media was changed at 24 hrs, and 48 hrs post transfection cells were irradiated (or not) with 10 Gy, incubated for an additional 1 hr, and proteins were extracted by autolyze on ice, using 1 mL of extraction buffer for each condition. Resuspended cells were sonicated, spun down at 14,000 rpm at 4° C. for 10 minutes. Cell extracts were rocked with 200 μL of resin (IgG anti-mouse or nanobody anti-GFP, immobilized on beads) for 2 hrs at 4° C. Beads were washed 4 times in 50 mM Tris-HCl (pH 8.0), 750 mM NaCl, 10% glycerol, and proteins bound were eluted in Laemmli buffer, loaded on gel, digested, and subjected to analysis by mass spectrometry (UTHSCSA Core facility). Data were visualized and analyzed using Scaffold.

### x. Affinity Pull-Down

Affinity pull-downs were performed as described elsewhere (E. Dray et al., Proc Natl Acad Sci USA 108, 3560-3565 (2011), E. Dray et al., Nat Struct Mol Biol 17, 1255-1259 (2010)). 2 μg (2.5 μL) of GFP-tagged EYA4 fusion protein immobilized on beads was incubated with 2 μg of purified proteins (H2A, H2B) in 10 μL of reaction buffer (25 mM Tris-HCl (pH 7.5), 120 mM KCl, 1 mM β-mercaptoethanol) for 30 minutes at 4° C. Unbound proteins were collected as supernatant (S). Beads were washed (wash W) three times with 100 μL of the same buffer and proteins complexed with EYA4 were eluted (E) in 20 μL of Laemmli buffer. 10 μL of the supernatant (S), first wash (W), and eluation (E) were analyzed by SDS-PAGE.

### xi. Cancer Cell Line Encyclopedia (CCLE) Analyses

Protein-level expression data from cell lines in the CCLE dataset with available EYA4 protein levels (n=90) were downloaded (D. P. Nusinow et al., Cell 180, 387-402 e316 (2020)). Chromosome instability (CIN70) and homologous recombination deficiency (HRD) scores in corresponding cell lines were determined as previously described (L. Carter, et al., Nat Genet 38, 1043-1048 (2006), G. Peng et al., Nat Commun 5, 3361 (2014)).

### xii. Chromosomal Break In Vitro Reporter System and Flow Cytometry

The HEK293-puro-DR-GFP cell line has been described elsewhere (P. Caron et al., Cell Rep 13, 1598-1609 (2015), J. O'Sullivan, et al., J Vis Exp 10.3791/62553 (2021)). Cells were reversed co-transfected with 3 μg I-SceI expression plasmid (pCOASce; (54)) and 500 ng MISSION TRC2 pLKO.5-Puro empty vector or shRNA constructs, as indicated. Transfected cells were kept in regular growth medium and analyzed by flow cytometry after 72 hrs to measure the percentage of cells expressing GFP (E. Dray et al., Proc Natl Acad Sci USA 108, 3560-3565 (2011)). For complementation, cells were co-transfected with shRNA1 and pcDNA3.1 Myc/His EYA4 or containing a mutant version of EYA4.

### xiii. Cell Cytotoxicity Assay (MTT)

HeLa cells (empty vector or knocked down for EYA4) were seeded in 96-well plates. After 24 hrs, increasing concentrations of PARP inhibitor (olaparib) were added. Cell cytotoxicity was measured following manufacturer's protocol (Abcam ab211091). Briefly, 50 μL serum-free media and 50 μL MTT reagent was added to each well and incubated at 37° C. for 3 hrs. MTT media was replaced with 150 μL of MTT solvent and incubated with agitation for 15 min. Absorbance was measured at 590 nm.

### xiv. Anti-GFP Nanobody Agarose Production

Plasmid encoding the sequence of the anti-GFP nanobody was obtained and the nanobody was purified (M. J. Schellenberg, et. al., Protein Sci 27, 1083-1092 (2018)). The protein was expressed in E. coli BL21 (DE3) strain with an induction with 0.1 mM of IPTG for 16 hrs at 16° C. Cells were harvested and resuspended in lysis buffer (50 mM Tris-HCl (pH 8.0), 1 M NaCl, 1 mM TCEP, protease inhibitors: aprotinin, chymostatin, leupeptin, and pepstatin A at 3 μg/ml each). The resuspended cells were sonicated with 5 cycles of 15 seconds with 30 seconds cooling periods (50% maximal output). Subsequently, lysed cells were clarified by centrifugation at 100,000×g for 45 minutes at 4° C., the supernatant was collected and incubated with 5 mL of Ni2+—NTA beads at 4° C. with agitation for 2 hrs. Beads were collected, washed with 200 mL of 50 mM Tris-HCl (pH 8.0), 1 M NaCl, and the protein eluted in 10×5 mL fractions in 50 mM Tris-HCl (pH 8.0), 150 mM NaCl, 300 mM imidazole. Fractions containing the nanobody, as identified by SDS-PAGE gel, were pooled, diluted in 50 mM Tris-HCl (pH 8) and passed over a 5 mL HiTrap Q fast flow column (Cytiva). The flow-through containing the unbound nanobody was concentrated to 5 mL and resolved onto a Hi load 16/200 Superdex 200 preparative column run in 0.1 M NaHCO₃ (pH 8.4) with 0.5 M NaCl. Fractions containing the purified nanobody were pooled together and used for the coupling reaction. Purified anti-GFP nanobody was coupled to cyanogen bromide-activated agarose (Sigma-Aldrich) following the manufacturer's indications. After coupling, blocking and washing, the beads were resuspended in 50 mM Tris-HCl (pH 8.0), 150 mM NaCl, 50% glycerol and kept at 4° C. until further use.

### xv. Protein Expression and Purification

Full length m-cherry EYA4 or the N-terminus domain of EYA4. Full length m-cherry EYA4 or the N-terminus domain of EYA4 (residues 1-365), was sub-cloned as a Strep-mCherry-EYA4-His construct in pFastBacl. Plasmids were used to make viral particles by first establishing a bacmid in DH10Bac (Invitrogen) then infecting Sf9 cells. 200,000 particles/ml were used to infect 400 ml of Sf9 cells (1.2×10⁶ cells/ml), grown 72 h at 27° C., and harvested by centrifugation (5 minutes 1000×g). Pellets were resuspended in lysis buffer (50 mM CAPS (pH 10.9), 100 mM NaCl, 20% sucrose, 10% glycerol, 0.5 mM EDTA, 1 mM TCEP, 1 mM PMSF, Igepal and protease inhibitors), sonicated 15 times for 20 seconds (50%). Lysed cells were clarified by centrifugation at 40,000×g for 40 minutes at 4° C. Supernatant was incubated with Ni-NTA²⁺ resin for 3 hr with agitation. The resin was washed with 10CV Buffer B, 1° C. V Buffer C+10 mM Imidazole, then eluted in buffer C+200 mM Imidazole. Elutions were then pooled, loaded onto a Q column, and eluted using a salt gradient 0-100% as described for the GFP-WT protein. Fractions containing EYA4 were pooled, dialyzed against buffer A to lower the conductivity, and loaded onto a monoQ column. The protein was eluted using a 15-60% gradient of buffer C. Fractions containing EYA4 were pooled, buffer exchanged in storage buffer (50 mM Tris-HCl (pH 8.0), 150 mM NaCl, 30% glycerol) and concentrated before storage at −80° C.

The C-terminus domain of EYA4 (residues 358-639). The C-terminus domain of EYA4 (residues 358-639), was sub-cloned as a GST-EYA4 (358-639) construct. The protein was expressed in E. coli C43 (DE3) strain with an induction with 0.1 mM of IPTG for 16 hrs at 16° C. Cells were harvested and resuspended in lysis buffer (50 mM Tris-HCl (pH 8.0), 150 mM NaCl, 20% sucrose, 10% glycerol, 0.5 mM EDTA, 1 mM TCEP, 1 mM PMSF and protease inhibitors), sonicated 10 times for 10 seconds. IGEPAL CA 630 was added (1% v/v), then lysed cells were clarified by centrifugation at 100,000×g for 45 minutes at 4° C. Clarified supernatant was then incubated with 5 mL affinity resin (Glutathione sepharose, Cytiva) for 90 min under agitation at 4° C. The resin was washed with 20 CV of buffer B, followed by 10 CV of buffer C. The protein was then eluted in 5×5 mL fractions in elution buffer D (50 mM Tris-HCl (pH 8.0), 150 mM NaCl, 25 mM glutathione, 10% glycerol). Fractions with EYA4 (358-639), as identified on SDS-Page, were pooled (approx. 25 mL), diluted with dilution buffer (50 mM Tris-HCl (pH 8), 10% glycerol) and loaded onto a Fast Flow Q column. The protein was fractionated in 3 mL fractions using a linear gradient to 100% of buffer B (50 mM Tris-HCl (pH 8.0), 1 M NaCl, 10% glycerol). Fractions containing the peak EYA4 were pooled and resolved onto a Superdex200 column run in Tris-HCl (pH 8.0) with 300 mM NaCl. Fractions containing EYA4 (358-639) were pooled together and concentrated before storage.

RAD51 WT and mutants cloned in pET11. RAD51 WT and mutants cloned in pET11 were expressed in BLR DE3 pLyS E. coli strain, and purified following previously described procedures (C. Wiese et al., Mol Cell 28, 482-490 (2007)). For GFP-RAD51 proteins, RAD51WT was a kind gift from Roland Kanaar (Erasmus); Y54F, Y315F and Y54D, Y315D were sub-cloned from pEt11d into pEGFP-C1 and all constructs were expressed in 293 suspension cells. Pellets from 250 ml suspension were resuspended in RAD51 lysis buffer (T. T. Paull, Curr Opin Genet Dev 71, 55-62 (2021).) containing complete Roche anti-proteases, homogenized in a Dounce homogenizer, sonicated 10 times at 50% for 20 seconds, and spun at 40,000×g for 30 minutes at 4° C. The clarified soluble proteins extract was loaded onto a 30 ml Fast Flow Q column and eluted with a 0-100% gradient of KCl in T buffer. Fractions eluted from 38-mM salt were combined, dialyzed against T buffer, and loaded onto a 5 ml macrohepatite column. Fractions containing RAD51 were pooled and incubated with 1 ml of GFP-nanobody agarose beads as described above. After 2 h incubation with agitation at 4° C., 400 μL of beads were collected, washed in T+1000, T+150, then resuspended in storage buffer (T+300 mM NaCl, 30% glycerol), aliquoted in 10 μL aliquots, and frozen at −20° C. to be used for pulldowns. The 600 uL beads left over were eluted by adding 200 mM Glycine pH2 in T+150 mM NaCl, to the resin and collected in 10 uL NaOH to neutralize the pH. Elutions were pooled, pH was adjusted to 9, diluted 1:1 in T buffer to lower the sample conductivity, and loaded onto a monoQ column. Fractions containing RAD51 were combined, concentrated, and frozen.

### xvi. DNA Substrates

Cy5-labeled oligo P1 (T. T. Paull Curr Opin Genet Dev 71, 55-62 (2021)). from IDT (TTATATCCTTTACTTTGAATTCTAT-GTTTAACCTTTTACTTATTTTGTATTAGCCGGATCCTTATTTCAATTATGTTCAT; SEQ ID NO:1) was used as the ssDNA substrate. dsDNA was generated by annealing Cy5-labeled oligo P1 with its exact complement P2 ATGAACATAATTGAAATAAGGATCCGGCTAATACA-AAATAAGTAAAAG GTTAAACATAGAATTCAAAGTAAAGGATATAA (SEQ ID NO:2). The annealed product was purified by SDS-PAGE then concentrated and quantified. For the long system assays, substrates were sourced from NEB. The ssDNA is the ϕX174 virion DNA and the dsDNA its replicative form ϕX174 RF I DNA.

### xvii. ATPase Assay

To measure ATPase activity, previously published protocols were followed (R. Prakash et al., Genes Dev 23, 67-79 (2009)).

### xviii. Mass Photometry

To investigate the oligomerization vs polymerization states of RAD51 species, samples were diluted to mass photometry analysis on (Reyfen). Proteins were diluted in EMSA reaction buffer containing ATP and MgCl₂ to 50 nM and the contrast was recorded over 60 seconds. For DNA binding analysis, DNA was added to the buffer+protein sample, allowed to equilibrate for 60 seconds, then recorded for 60 seconds. Covalbumin (75 kDa), Aldolase (158 kDa) and Ferritin (440 kDa) were used to establish the mass calibration.

Altematiely, samples were diluted to mass photometry analysis on (Reyfen). Proteins were diluted in Buffer A or reaction buffer. Covalbumin (75 kDa), Aldolase (158 kDa) and Ferritin (440 kDa) were used to establish the mass calibration.

### xix. EYA4-Deficient Cells Accumulate DNA Damage

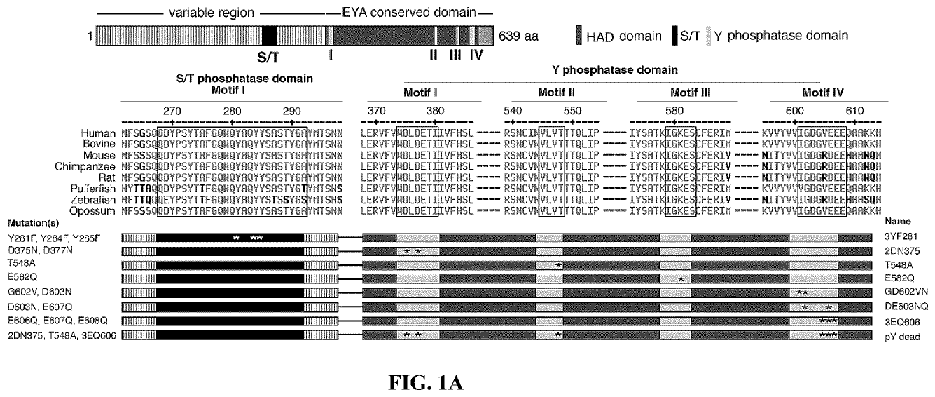

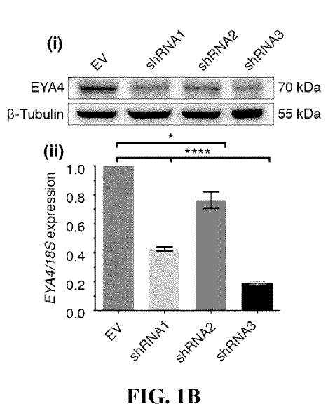

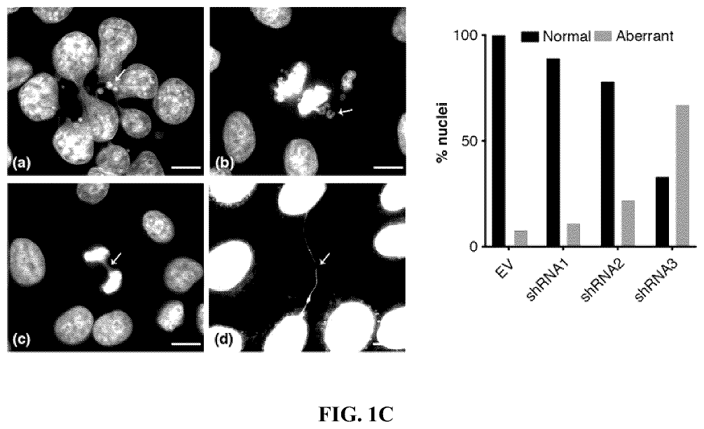

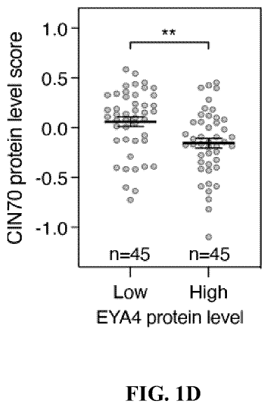

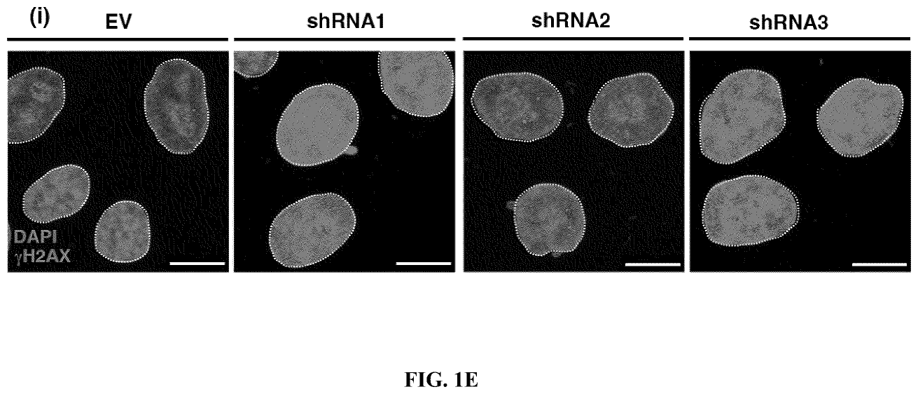

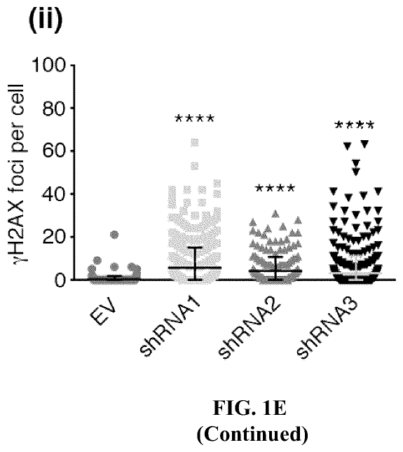

Complete knockout of EYA4 is lethal in most mouse strains shortly after birth (F. F. Depreux et al., The Journal of Clinical Investigation 118, 651-658 (2008)), and poorly tolerated in several lung cell lines (I. M. Wilson et al., Oncogene 33, 4464-4473 (2014)) and other cell lines. However, using short hairpin RNAs (shRNAs) a significant decrease EYA4 expression and protein levels in HeLa cells is observed (FIG. 1B), HEK-293T cells, and MDA-MB-231 cells, and mutants of the EYA4 catalytic domains were established (see FIG. 1A for details).

Referring to FIG. 1A, a representative depiction illustrating that two domains of EYA4 that possess phosphatase activity are well conserved among vertebrates is shown. Mutations used in this study are indicated by stars. FIG. 1B shows representative data illustrating that incomplete knock down can be achieved by shRNA, and leads to decrease of both proteins.

Upon knocking down EYA4, HeLa cells readily acquired nuclear defects indicative of genomic instability (FIG. 1C). Cells depleted for EYA4 showed aneuploidy, DNA bridges, and accumulation of micronuclei. This is consistent with various mitotic defects, particularly in metaphase, previously observed upon EYA4 knockdown in Mitocheck screens (Table 2)(Y. Cai et al., Nature 561, 411-415 (2018)). To further assess this phenotype in a broader panel of cell lines, 90 cell lines were identified in the Cancer Cell Line Encyclopedia (CCLE) dataset with available EYA4 protein-level data (D. P. Nusinow et al., Cell 180, 387-402 e316 (2020)). The CIN70 signature was used to evaluate the degree of genomic instability (S. L. Carter, et al., Nat Genet 38, 1043-1048 (2006)). Cell lines with low EYA4 protein levels showed significantly higher protein expression levels of CIN70 signature proteins, a surrogate for the degree of genomic instability (Y. Cai et al., Nature 561, 411-415 (2018)), when compared to cell lines with high EYA4 protein levels (p=0.0028, t test) (FIG. 1D).

Referring to FIG. 1C, representative data illustrating that cells depleted for EYA4 present with large numbers of nuclear aberrations (bridges, micronuclei, lost chromosomes; quantification N>300) is shown. FIG. 1D representative data illustrating that EYA4 depletion leads to elevated γH2AX foci (i) microscopy and (ii) quantification is shown.

TABLE 2
Mitocheck Primary Screen

| Screen ID | DSR00029388 |
|---|---|
| Library Name | Silencer Pre- |
| | designed siRNA |
| Company | Ambion |
| Phenotypes | Observed with |
| | x/y EYA4 siRNAs |
| Grape | 1/4 |
| Mitotic delay/arrest | 2/4 |

Mitocheck Validation Screens

| | | |
|---|---|---|
| Screen ID | DSR00054740 | DSR00054741 |
| Library Name | Silencer Select | Silencer Select |
| | Pre-designed siRNAs | Pre-designed siRNAs |
| Company | Ambion | Ambion |
| Validation Screen | Validation screen 1 | Validation screen 2 |
| Number | | |
| Phenotypes | Observed with | Observed with |
| | x/y EYA4 siRNAs | x/y EYA4 siRNAs |
| Metaphase alignment | 1/4 | 3/4 |
| problems/including no | | |
| metaphase | | |
| Metaphase delay/arrest | 1/4 | 3/4 |
| Segregation | 0/4 | 1/4 |
| problems/chromatin | | |
| bridges/lagging | | |
| chromosomes/multiple | | |
| DNA masses | | |
| Strange nuclear shape | 1/4 | 0/4 |

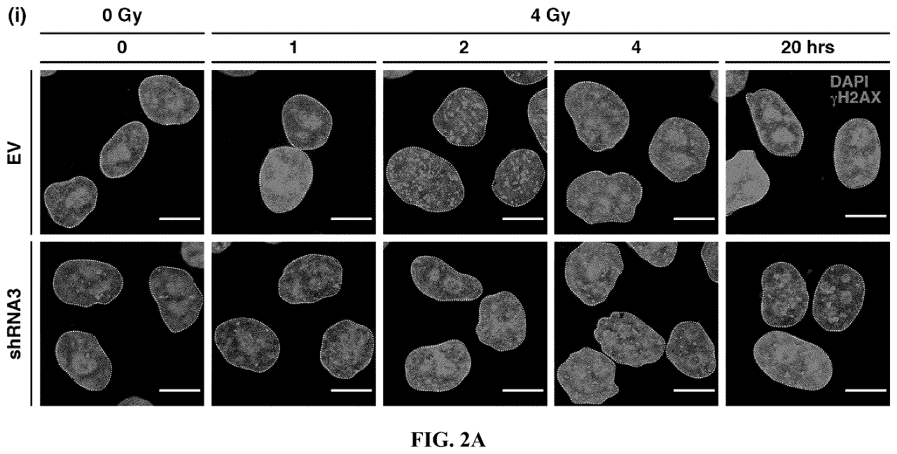

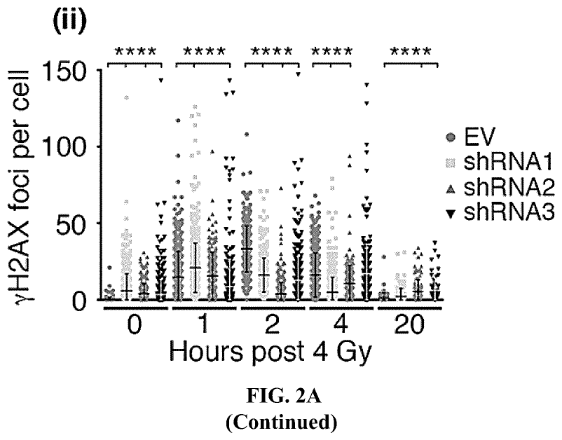

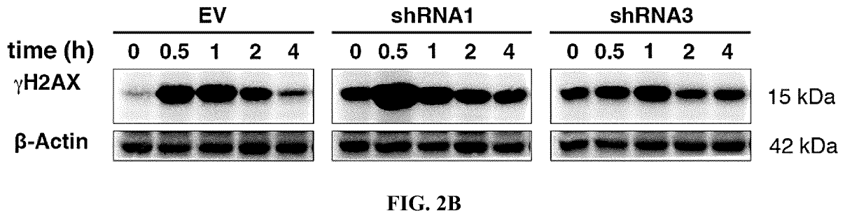

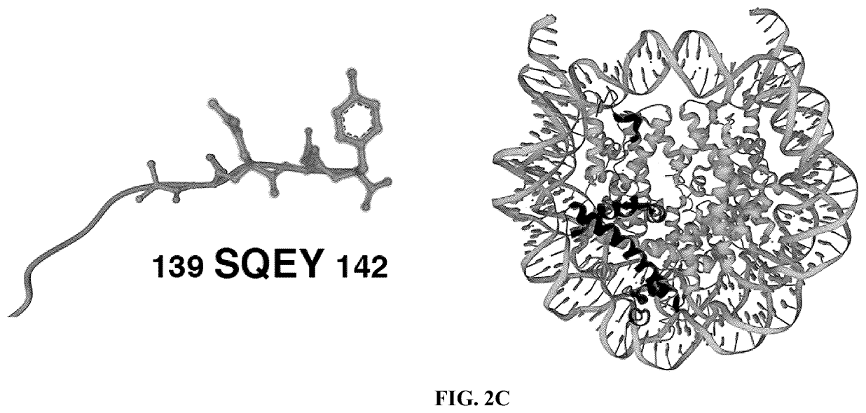

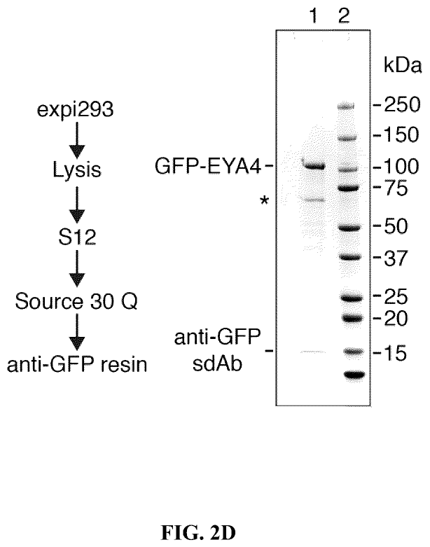

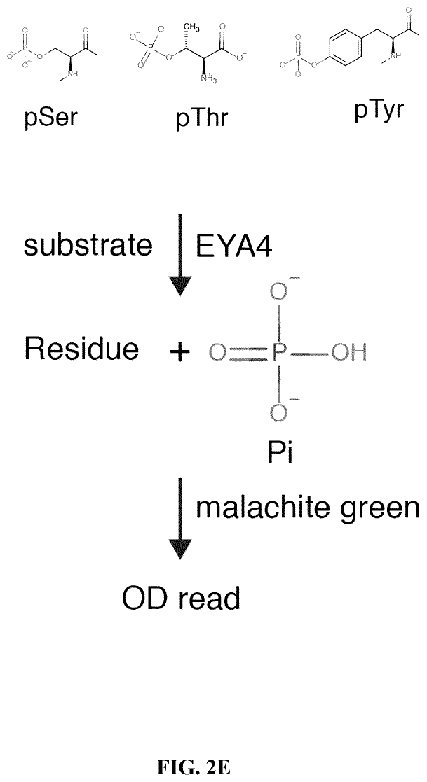

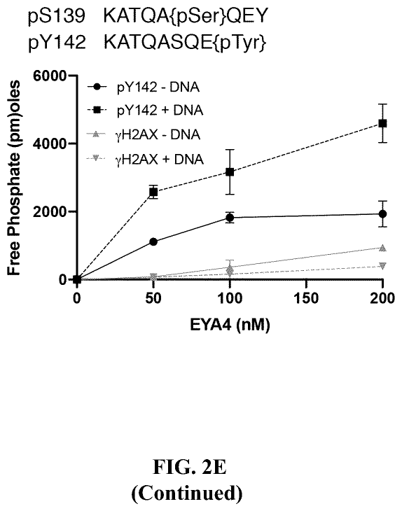

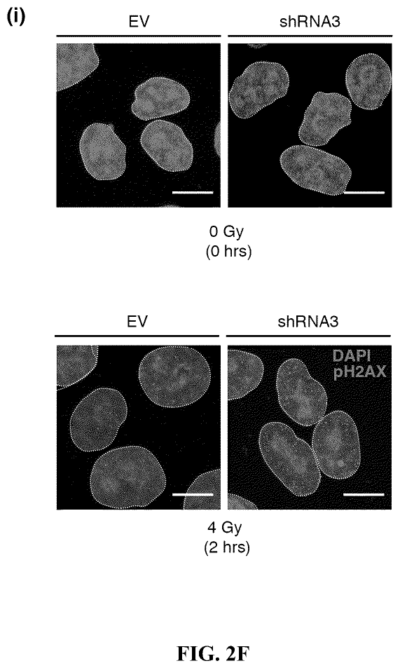

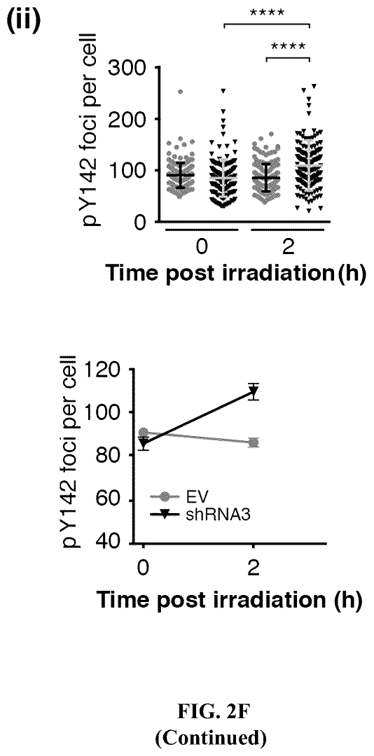

The level of γH2AX was investigated in control cells and cells depleted for EYA4 by any of three shRNAs. When compared to cells expressing a non-targeting shRNA, more than 50% of EYA4-depleted cells exhibited an overall 8-fold increase of γH2AX foci in normal growth conditions (5% CO₂, 37° C.) without any exogenous stress and contained 10 or more discrete γH2AX foci (FIG. 1E). Accumulation of γH2AX is a reliable marker for DSBs, however there is the possibility that H2AX S139 could be a target of EYA4 phosphatase activity and thus remain phosphorylated regardless of the DNA repair status, even in the absence of EYA4. A test for whether EYA4-depleted cells accumulate unrepaired DNA breaks or whether they are proficient for repair but deficient for dephosphorylating H2AX was created by causing DSBs with irradiation, then followed the stimulation and resolution of γH2AX foci. A time course of γH2AX foci by immunofluorescence (FIG. 2A) and measurement of protein levels by Western blotting (FIG. 2B) both show that γH2AX is constitutively elevated in cells depleted for EYA4, but still increases following irradiation. Interestingly, some DSBs are repaired in EYA4-depleted cells because γH2AX is resolved over time and returns to its initial, elevated levels, 2-4 hours post irradiation (FIG. 2A and FIG. 2B). These observations demonstrate that EYA4 depleted cells exhibit accumulation of DNA damage but repair, at least partially, some irradiation-induced DSBs.

Referring to FIG. 1E, representative data illustrating the CIN70: chromosomal instability score based on expression levels of 70 proteins, showing CIN linked to EYA4 expression is shown. 90 cell lines from CCLE split in two equal-size groups: EYA4 low and EYA4 high with median EYA4 protein expression level used as cut-off. Means+/−SEM are shown. P value: p=0.0028, t test. FIG. 2A, representative data illustrating the kinetics of γH2AX foci formation over time, in cells proficient and deficient for EYA4, and after irradiation is shown. Specifically, in (i) representative images are shown and in (ii) foci are quantified and plotted+/−standard deviation. FIG. 2B shows representative data illustrating γH2AX protein levels followed by Western blot after irradiation (10Gy).

### b. Histone Variant H2AX pY142 but not pS139, is a Substrate of the EYA4 Phosphatase Activity

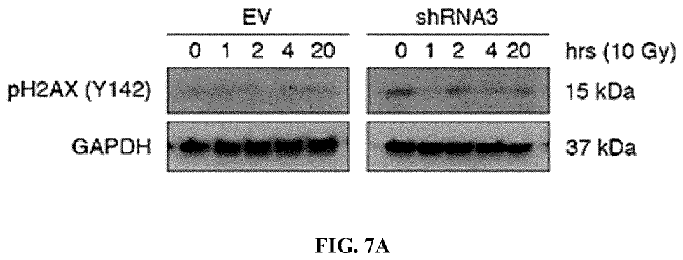

PP4 and WIP1 are the known phosphatases for γH2AX (D. Chowdhury et al., Mol Cell 31, 33-46 (2008) and H. Cha et al., Cancer Res 70, 4112-4122 (2010)) and EYA4 is unlikely to dephosphorylate S139, but EYA1-3 proteins have been found directly or indirectly to dephosphorylate residue Y142 of H2AX upon DNA damage (R. S. Hegde, et al., Crit Rev Biochem Mol Biol 55, 372-385 (2020), I. M. Wilson et al., Oncogene 33, 4464-4473 (2014)), and by similarity, EYA4 was suggested to possess the same activity (I. M. Wilson, et al., Oncogene 33, 4464-4473 (2014)). The two residues are in close proximity (FIG. 2C) and while pY142-H2AX is a low abundance modification (A. A. Hatimy, et al., International Journal of Mass Spectrometry 391, 139-145 (2016).), it is thought to be essential for the promotion of DNA damage repair over apoptosis (P. J. Cook et al., Nature 458, 591-596 (2009), S. Solier, et al., Cell Cycle 8, 1853-1859 (2009)). The dephosphorylation of H2AX by EYA4 was investigated, EYA4 was expressed and purified as a GFP-fusion protein using previously described chromatography conditions (FIG. 2D). Purified EYA4 was incubated with phosphorylated H2AX peptides (FIG. 2E, and Table 3) containing either pS139 or pY142 at 37° C. for 60 min. Released phosphate was quantified by a colorimetric method using malachite green-molybdate (FIG. 2E). Both threonine and tyrosine phosphatase domains of EYA4 have been described as functional in vitro (Y. Okabe, et al., Nature 460, 520-524 (2009)). Under conditions optimal for EYA4 tyrosine phosphatase activity (acidic, with Mg²⁺), the synthetic pY142 peptide was dephosphorylated 5-fold more efficiently than pS139 peptides (FIG. 2E). Using the serine threonine-optimized conditions (described in supplemental methods), pS139 was still minimally dephosphorylated and pY142 was not dephosphorylated, indicating that the experimental conditions are specific for each of the activities tested. EYA4 is not the phosphatase for γH2AX but its efficient dephosphorylation of H2AX on pY142 could promote DNA damage repair signaling following DNA damage. The results show that the phosphatase activity of EYA4 is greatly stimulated by addition of single-stranded DNA in the reaction (+/−DNA, FIG. 2E). Verification of the results was obtained of the in vitro findings in cells, using a specific antibody directed against pY142 of H2AX, for immunofluorescence and in Western blots. Higher levels of pY142 were detected in EYA4 depleted cells compared to controls (FIG. 2F and FIG. 7A). Y142 phosphorylation level decreases in response to DNA damage (P. J. Cook et al., Nature 458, 591-596 (2009)), but also an accumulation of pY142-H2AX in EYA4 depleted cells, at 2 hours following irradiation (FIG. 2F and FIG. 7A). Without wishing to be bound by theory, this suggests that EYA4 could target H2AX pY142 for dephosphorylation, but also contributes to its accumulation, possibly by targeting upstream kinases.

Referring to FIG. 2C representative data illustrating that residues S139 and Y142 are on the extreme C-terminal position of the histone tail, H2AX (PDB P16104) is depicted in darker shade on a nucleosome structure is shown. FIG. 2D shows representative data illustrating a protein purification scheme (left) used to purify GFP-EYA4. SDS PAGE and Coomassie stain (right) shows the purified protein. FIG. 2E shows representative data illustrating a schematic of the malachite green colorimetric assay (left) used to investigate possible substrates of EYA4 (right) such as residues pS139 and pY142 in H2AX. FIG. 2F shows representative data illustrating the Foci formation in EYA4 depleted cells using an antibody specific of phospho-tyrosine 142.

TABLE 3

| Protein | Residues | Peptide |
|---|---|---|
| pH2AX | Tyrosine 142 | KATQASQE{pTYR} |
| γH2AX | Serine 139 | KATQA{pSER}QEY |
| RAD51 | Tyrosine 54 | VA{pTYR}APKKEL |
| RAD51 | Tyrosine 315 | TRICK{pTYR}IDS |

### c. EYA4 is a DNA Binding Protein and Interacts with DNA, Histones and Nucleosomes

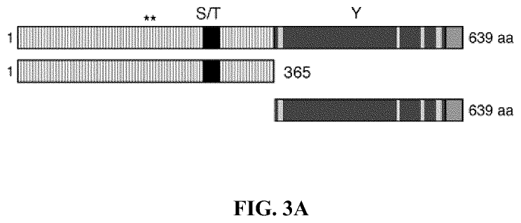

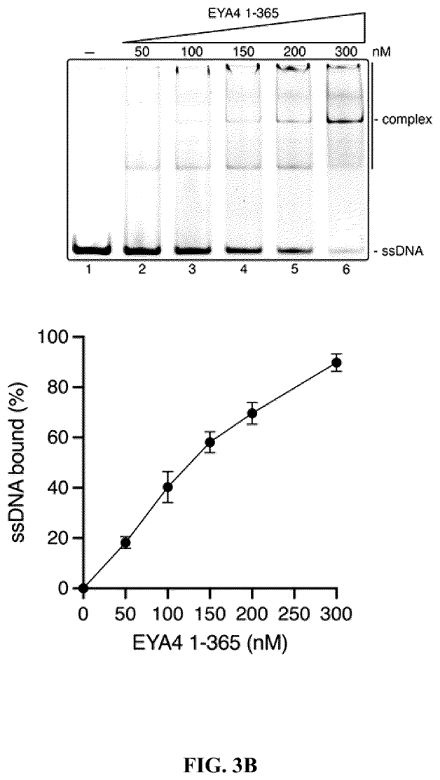

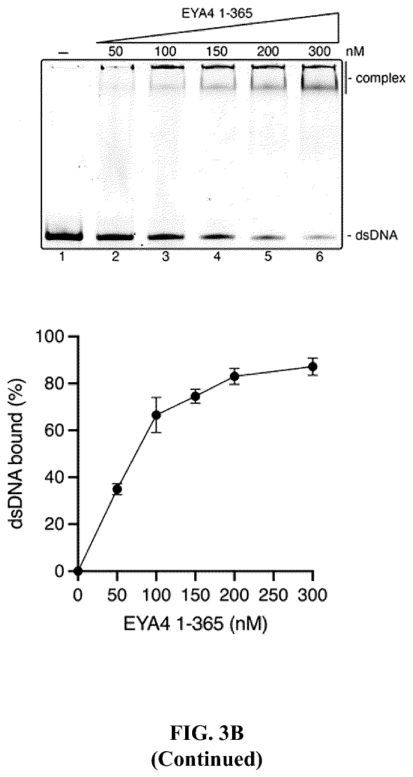

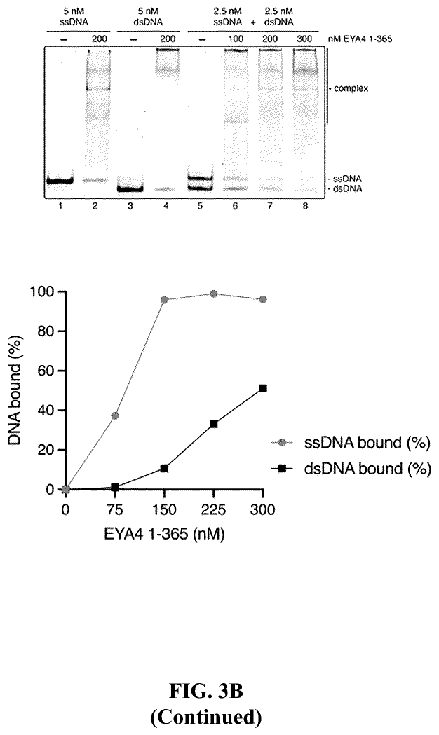

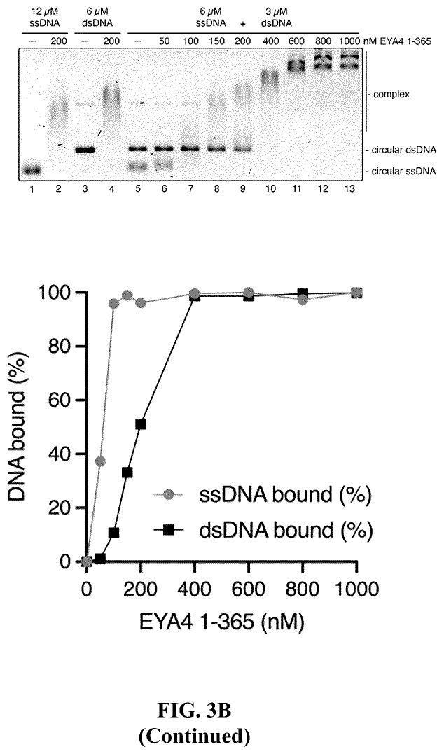

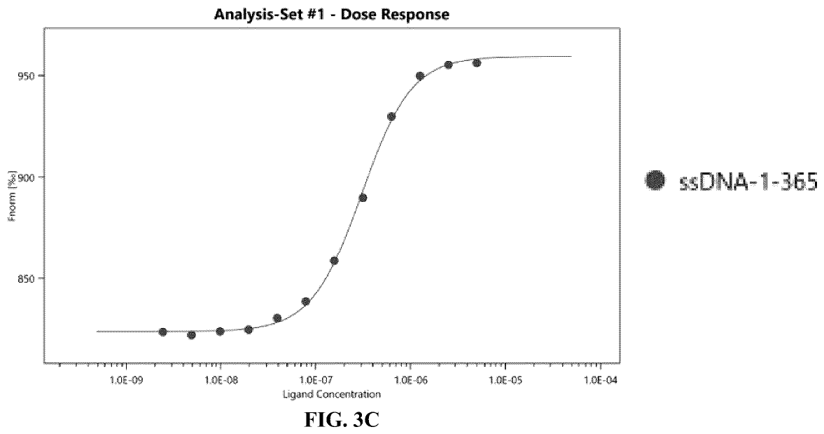

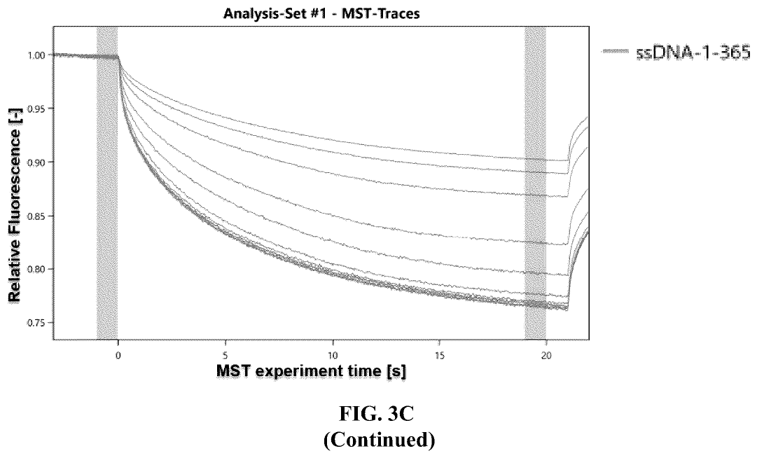

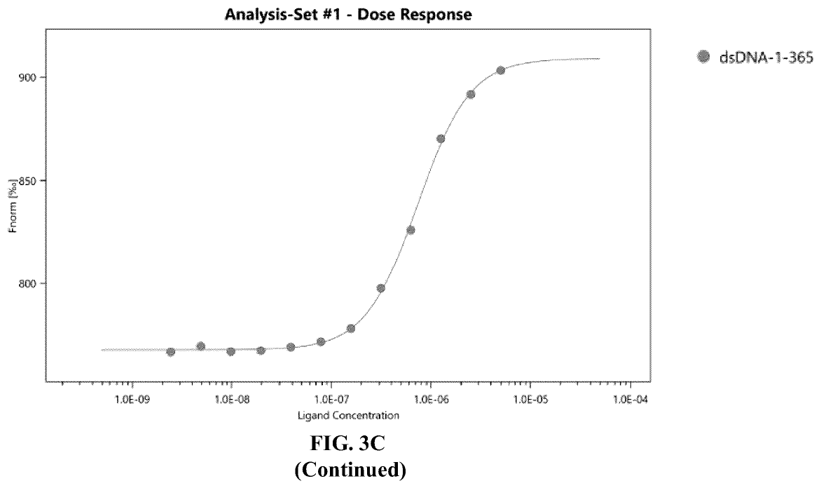

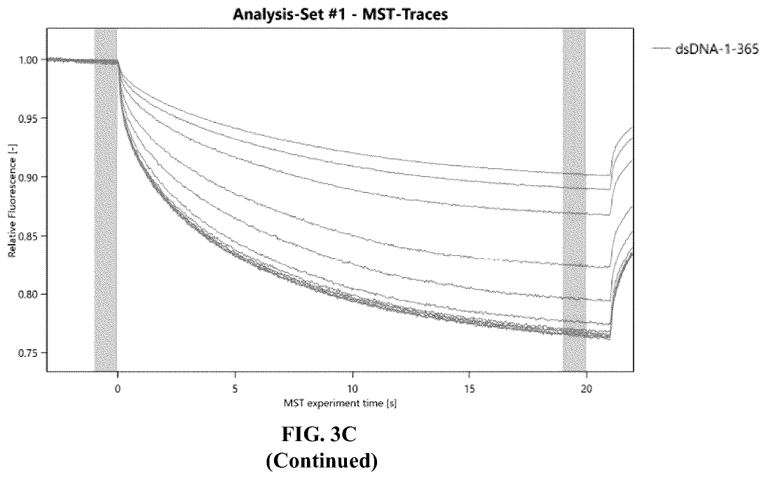

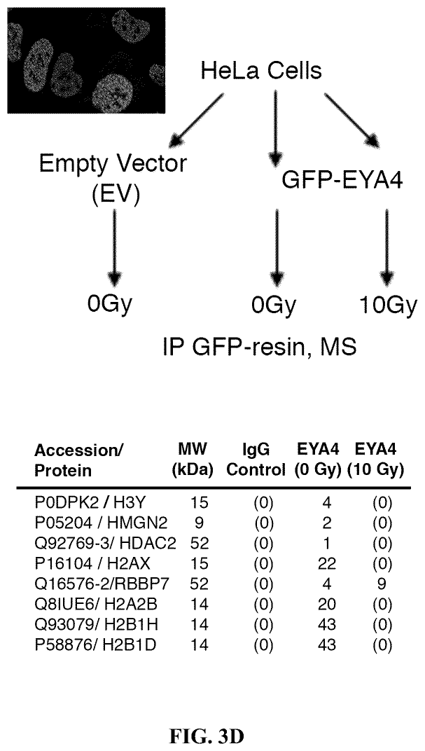

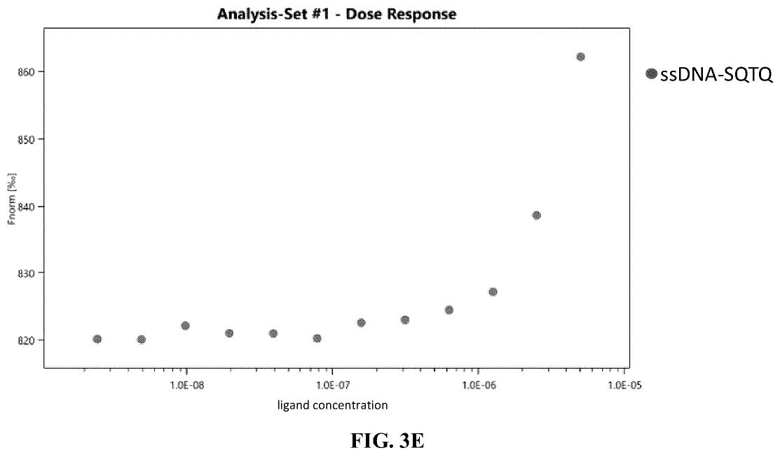

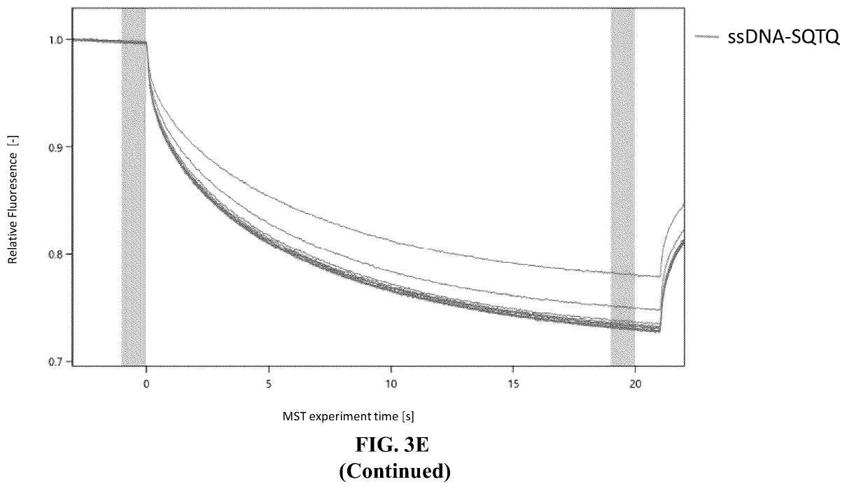

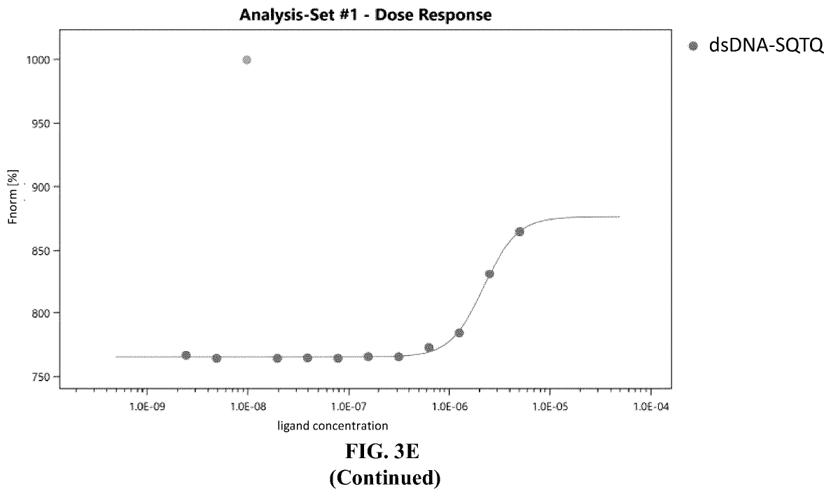

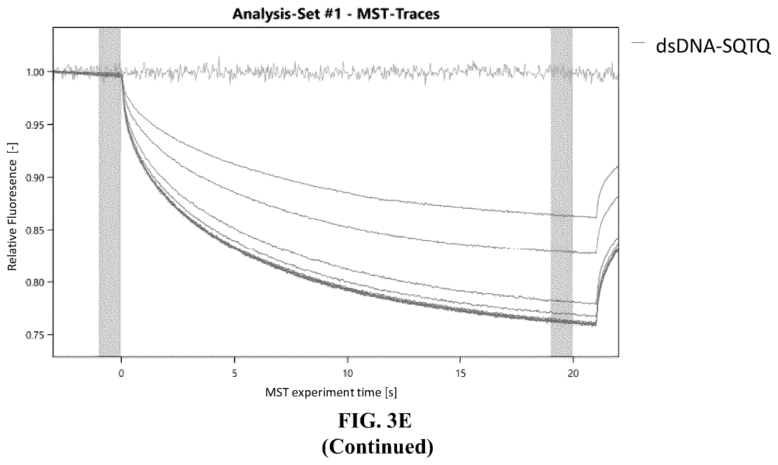

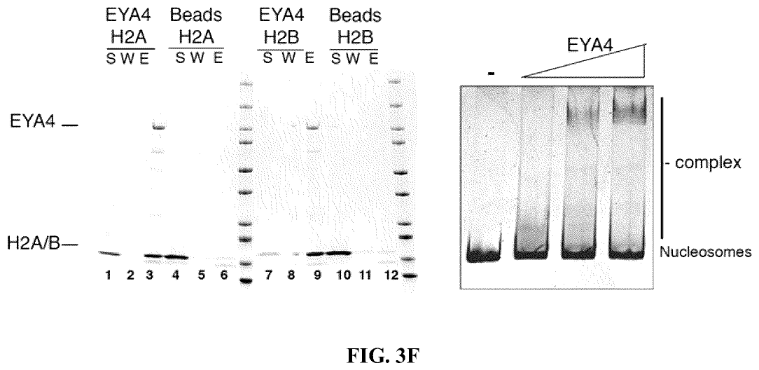

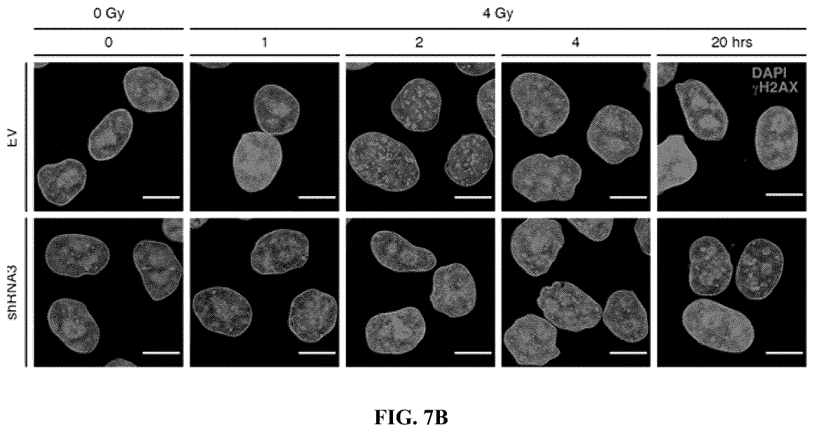

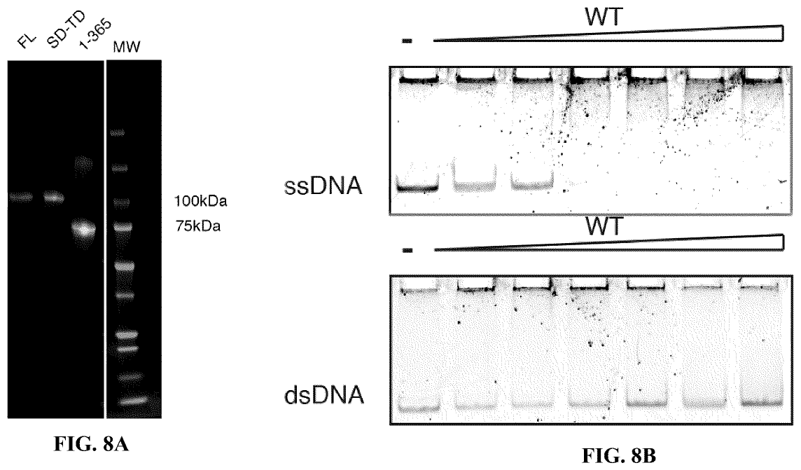

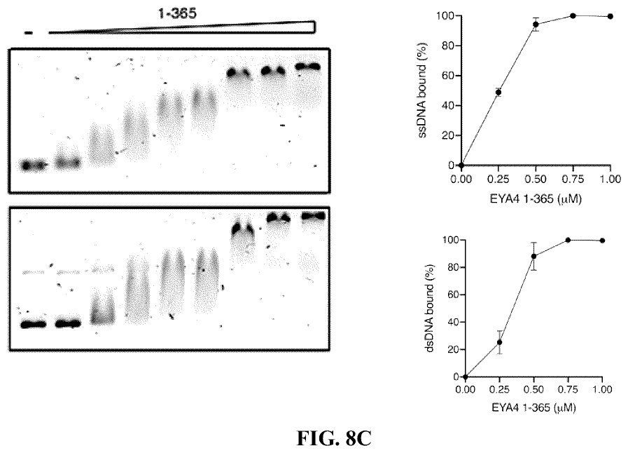

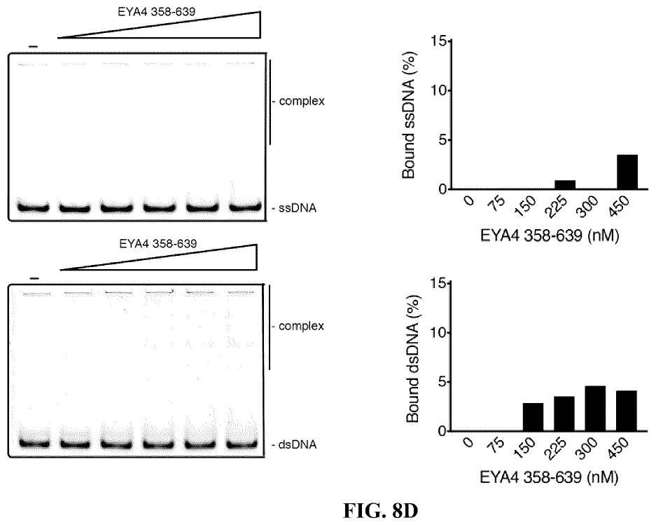

The unexpected stimulation of phosphatases reaction by DNA raised the possibility that EYA4 could interact directly with DNA. Purified EYA4, GFP full length (FIG. 2D), N-terminally m-cherry-tagged EYA4 (aa 1-365, FIG. 3A and FIG. 8A), and GST-C-terminal EYA4 (aa 358-639; FIG. 8A) was used to explore this possibility. Electromobility shift assay (EMSA) using purified N-EYA4 (1-365) and synthetic substrates to mimic ssDNA or dsDNA was performed. It was found that EYA4 binds to both ssDNA and dsDNA (FIG. 3B and FIG. 8B) and in a competition, identified substrate specificity for ssDNA. The N-terminus of EYA4 contains the DNA binding activity and exhibits the same substrate specificity as full-length EYA4, namely preferring ssDNA over dsDNA using a “short system” where substrates are purified oligonucleotides (FIG. 3B) or a “long system”, which utilizes intact plasmids with no DNA end available (FIG. 3B). The C-terminus EYA4, encompassing the tyrosine domain (358-639) does not possess DNA binding activity (FIG. 8D). Super-shifts observed with increasing concentration of EYA4 in both the short and long systems indicate that it accumulates on the phage DNA and does not bind solely to DNA ends. Dephosphorylation of H2AX and DNA binding activity makes EYA4 a good candidate for a role in DNA repair. Immunoprecipitation was performed followed by mass spectrometry in HeLa cells expressing endogenous levels of EYA4 and subjected to either no irradiation or 10 Gy irradiation. Among other interactions, EYA4 was found strongly associated with chromatin, and bound to histones and associated proteins in the absence of IR. Binding to histones is weakened or lost following irradiation (FIG. 3D), suggesting that EYA4 could be an early responder to DNA damage and is displaced following dephosphorylation of H2AX, possibly to allow chromatin accessibility by repair machineries. Looking at EYA4 sequence, ATM-ATR consensus site SQTQ in position 209-212 was identified. Mutations were introduced to create a phosphomimetic EYA4 double mutant: S209D, T21 ID. The DNA binding experiments were repeated, and observed a near complete abolishment of the DNA binding activity in the double mutant (FIG. 3E and TABLE 4). This partially explains the observation that EYA4 relocates from the chromatin following DNA damage, likely upon phosphorylation by ATM. Since EYA4 binds to DNA, it was to be determined if the histones binding observed in IP is direct or through DNA. Using the GFP-EYA4 protein immobilized on GFP-nanobody beads (M. J. Schellenberg, et al., Protein Sci 27, 1083-1092 (2018)) and purified histones, in vitro pull-downs were performed, and extremely robust interactions between EYA4 and H2A or H2B were identified (FIG. 3F). Despite its preference for ssDNA, EYA4 also binds strongly to dsDNA nucleosomes (FIG. 3F).

Referring to FIG. 3A representative data illustrating FL WT, or FL Mutated (** indicate S209, T211residues), N-terminal 1-365 or C-terminal construct were purified for testing DNA binding activity is shown. FIG. 3B shows representative data illustrating increasing concentrations (0-300 nM) of EYA4 1-365 were incubated with ssDNA, dsDNA or a mixture of both. Electromobility shift assay was visualized by tracking the Cy5-DNA on gel under UV (Biorad imager). The percentage of bound DNA was quantified and plotted. Error bars=mean±SEM of n>3. FIG. 3C shows representative data illustrating MST experiment shows binding of EYA4-N to ssDNA binding (KD 303 nM) and dsDNA (KD 732 nM). FIG. 3D shows representative data illustrating IP-MS workflow and a subset of peptides pulled-down with GFP-EYA4 in HeLa cells non-irradiated (0 Gy) or 1 hr after irradiation (10Gy). FIG. 3E shows representative data illustrating a mutant of EYA4 that mimics phosphorylation on residues S209 and T211 does not bind DNA as shown by MST (KD>2 μM). FIG. 3F shows in vitro pull-down with purified histones H2A and H2B(left) or nucleosomes (NEB kit). FIG. 8A shows M-cherry constructs (FL WT, FL (S209D, S211D), and 1-365) or a GST(359-639) construct were expressed and purified to test DNA binding properties of EYA4. FIG. 8B shows EYA4 full length WT was found to bind ssDNA using phage DNA. FIG. 8C shows that using the 1-365 construct, a comparable ssDNA binding activity was observed, and at concentration>500 nM 1-365 also binds to dsDNA. FIG. 8D shows conversely, the 358-639 fragment does not possess significant DNA binding activity, for ssDNA nor dsDNA.

TABLE 4

| Parameter | ssDNA 1-365 | | ssDNA SQTQ | | dsDNA 1-365 | | dsDNA SQTQ | |
|---|---|---|---|---|---|---|---|---|
| Unbound | 823.91 | | 736.31 | | 767.82 | | 765.29 | |
| Bound | 959.47 | | 902.77 | | 908.91 | | 876.46 | |
| EC₅₀ | 303.45 | nM | 514.95 | nM | 732.85 | nM | 2197.1 | nM |
| EC₅₀ | ±10.7044 | nM | ±18.1 | nM | ±26.3085 | nM | ±97.0 | nM |
| Confidence | | | | | | | | |

| | | | | |
|---|---|---|---|---|
| Hill Coeff. | 1.6539 | 2.662 | 1.6489 | 2.6104 |
| Signal to Noise | 74.3 | 59.0 | 91.6 | 75.8 |
| Resp. Amplitude | 135.5586 | 166.4557 | 141.0935 | 111.17 |

### d. EYA4 is Part of DNA Damage Repair Foci

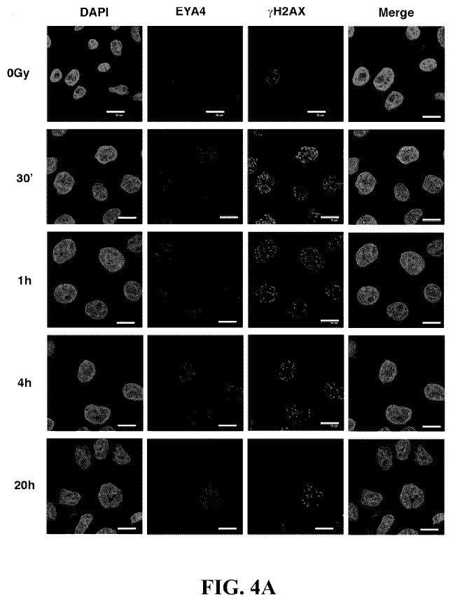

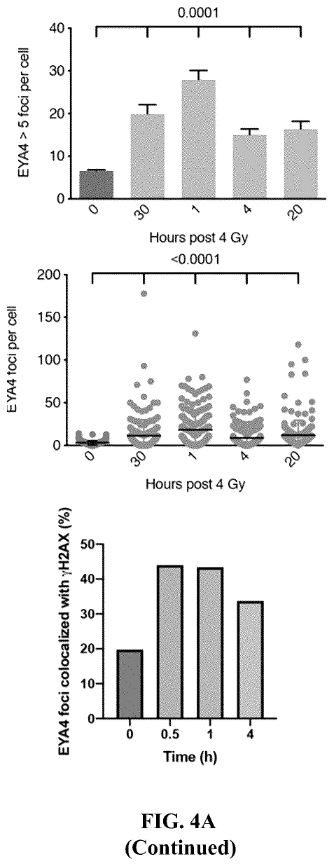

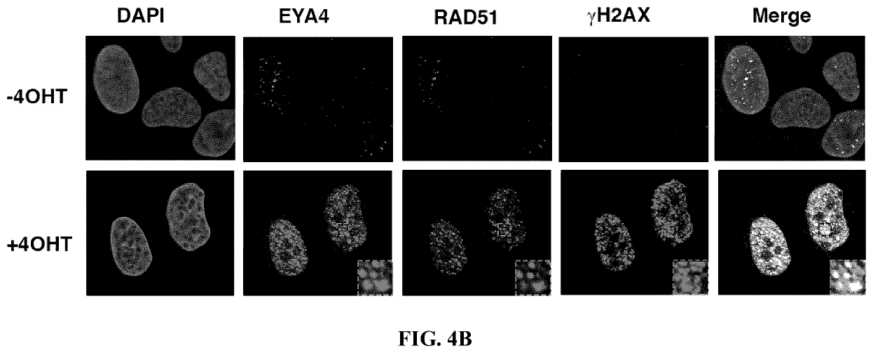

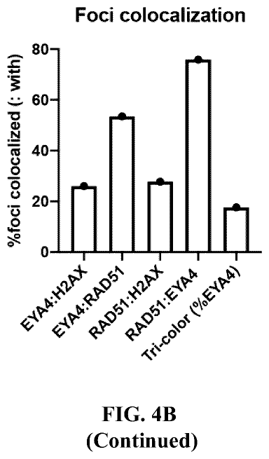

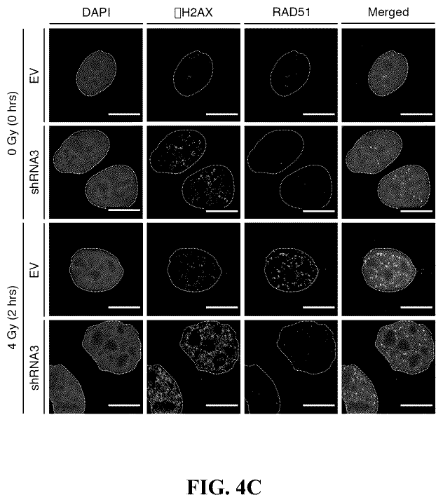

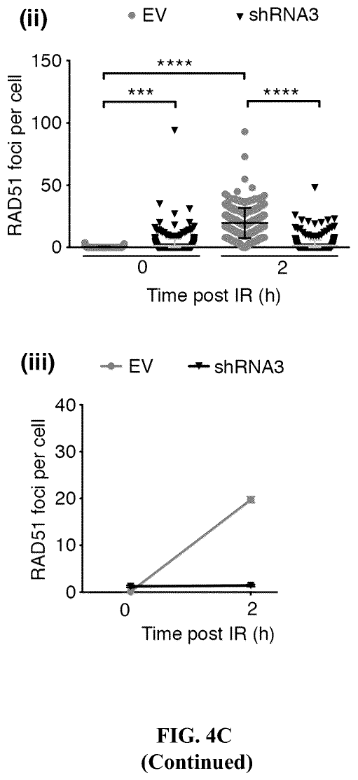

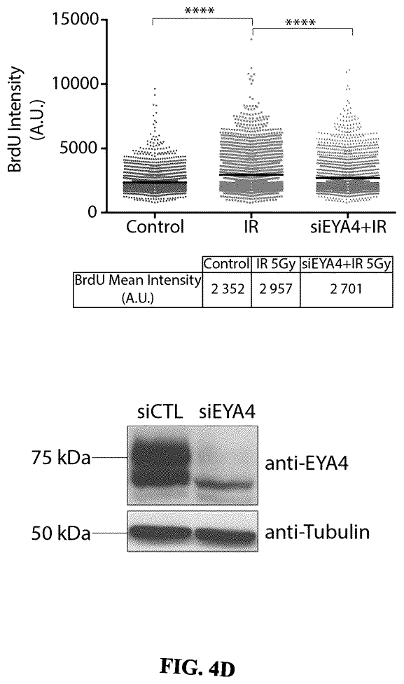

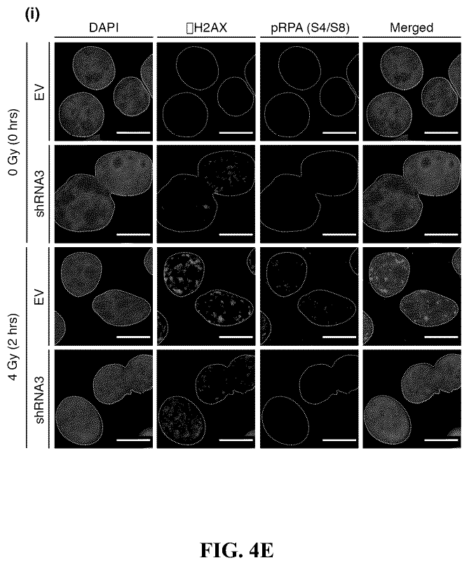

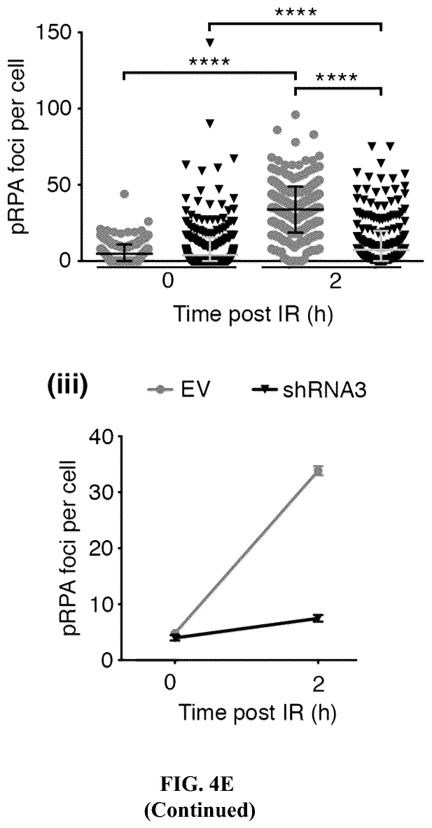

To gain a better understanding of EYA4 cellular behavior, the GFP-tagged version of EYA4 in cells was followed over time. EYA4 forms spontaneous foci in cells. Untreated cells possess an average of 20, small discrete EYA4 foci, that appear randomly distributed in the nucleus (FIG. 3D) and fuse into bigger structures in G2 phase. When using an antibody directed against the endogenous EYA4 in WT cells, it was found that irradiation promptly stimulates EYA4 foci formation, and these colocalize with γH2AX (FIG. 4A). Further interrogation of the colocalization of EYA4 with other DNA repair proteins, but also the utilization of a different system to confirm that the observations are not due to the irradiation or the antibody was performed using the DIvA system (DSB inducible via AsiSI (P. Caron et al., Cell Rep 13, 1598-1609 (2015)), provided by the Legube lab, transfected with GFP-EYA4, probing for the GFP tag during immunolocalization. In untreated DIvA cells, a low basal level of γH2AX foci, and low or absent RAD51 foci were observed (FIG. 4B, top panel). Cells expressing highest levels of EYA4 were also found to exhibit RAD51 foci, even in the absence of γH2AX (FIG. 4B, top panel). Without wishing to be bound by theory, this suggest that EYA4 co-localizes with non-repair RAD51 foci, and that RAD51 accumulates in the presence of EYA4. Following the addition of tamoxifen and subsequent translocation of AsiSI, DSBs were introduced as shown by increased γH2AX, and EYA4 formed robust foci, a large proportion of which colocalize with RAD51 foci (54%, FIG. 4B, bottom panel) and with γH2AX (25%).

Referring to FIG. 4A shows representative data illustrating EYA4 (2^{nd} column) and γH2AX (3^{rd} column) foci were observed by indirect immune-fluorescence on samples fixed after no irradiation (OGy) or 4 Gy irradiation, at the indicated time points. Foci were quantified and plotted. FIG. 4B shows representative data illustrating GFP-EYA4 transfected DIVA cells, with no DSB (−4OHT) or after induction of DSB by hydroxytamoxifen (+4OHT) were imaged and co-localization of EYA4, RAD51, and γH2AX at the break was quantified and plotted.

### e. In the Absence of EYA4, Homologous Recombination Efficiency is Compromised

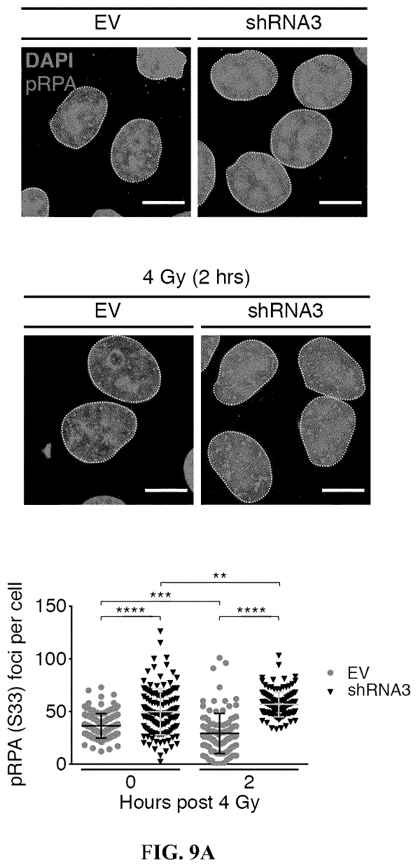

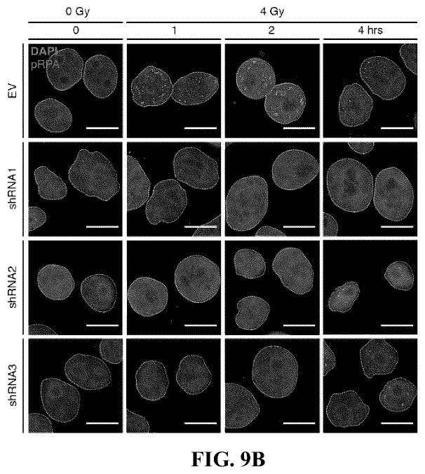

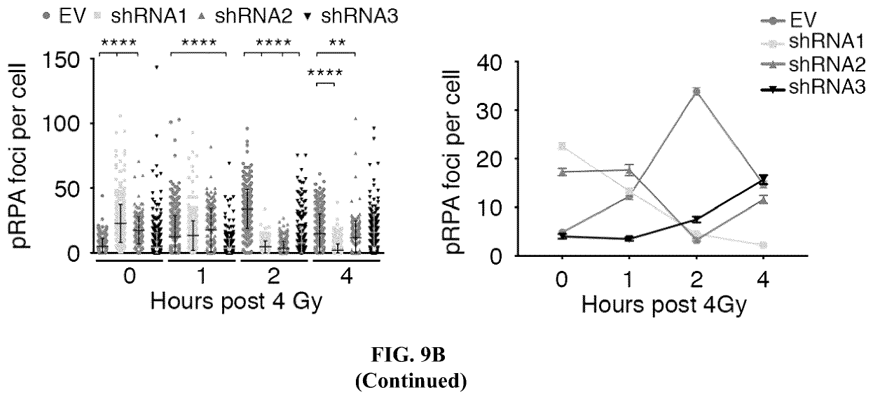

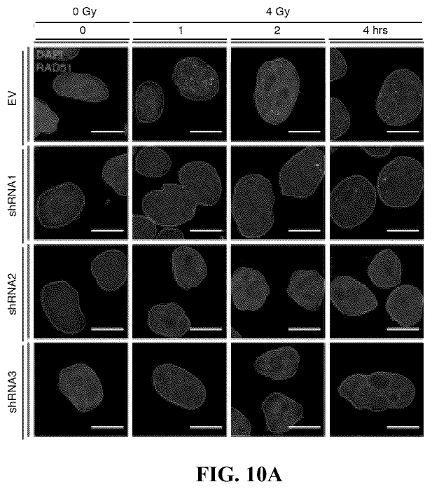

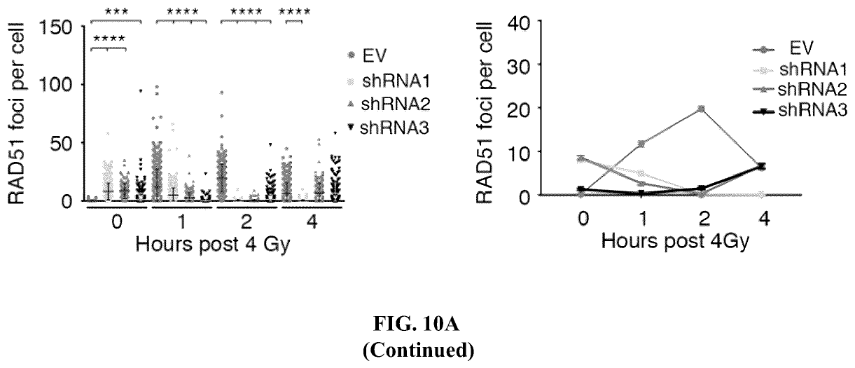

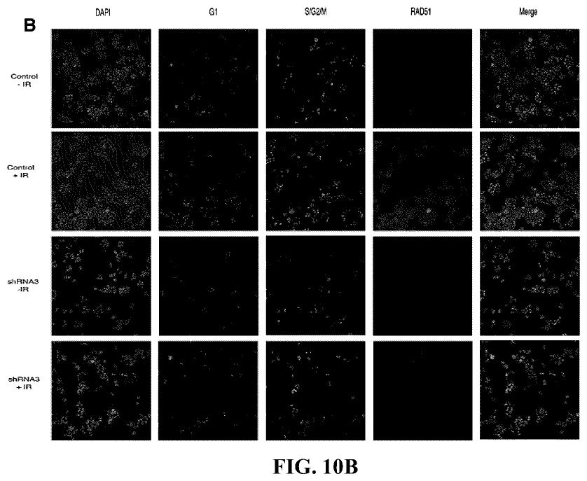

Accumulation of unrepaired DSBs in EYA4-depleted cells and the colocalization of EYA4 with γH2AX and RAD51 indicate that EYA4 might be a hitherto unidentified player of the DNA damage repair response. The repair of DSBs in the absence of EYA4, after no or 4Gy irradiation was investigated. Ionizing radiation-induced foci formation by γH2AX, pRPA, and RAD51 was followed. EYA4 depleted cells were found to accumulate γH2AX foci and replicative pRPA (phosphorylated at residue S33; FIG. 9A) but failed to form pRPA (S4/S8) foci that are surrogate markers of end resection at the break (FIG. 4C and FIG. 9B). This suggests that the signaling of the break is perturbed in the absence of EYA4 and processing of the break might not be fully proficient, which was verified using a previously published protocol, where incorporation of BrdU is measured in native conditions (J. O'Sullivan, et al., J Vis Exp 10.3791/62553 (2021)). The lack of a denaturing step allows to measure exclusively ssDNA, as a readout for resection efficiency. Use of this technique provided evidenced that cells depleted for EYA4 exhibit diminished resection compared to control cells, in response to IR (FIG. 4D). Consistent with this, RAD51 is recruited less efficiently to the break in EYA4 depleted cells, and later than in control cells (FIG. 4E and FIG. 10A). Using cells stably expressing the Fucci system (R. Tropee et al., Breast Cancer Res Treat 185, 601-614 (2021)), in which clover-geminin and mKO-cdt1 label cells in green when in S-G2-M and in red when in G1 respectively, it was verified that EYA4 depleted cells present no significant defect in cell cycle that would explain decreased RAD51 foci formation (FIG. 10B).

Referring to FIG. 4C representative data illustrating γH2AX and pRPA foci (S4/S8) were observed overtime, after no (0 Gy) or 4 Gy irradiation, in controls (EV) or cells depleted for EYA4 (shRNA3) is shown. Foci were imaged (i) and quantified (ii). FIG. 4D shows representative data illustrating BrdU incorporation in control cells or silenced for EYA4 after irradiation allows indirect measurement of resection. FIG. 4E shows representative data illustrating that the same experiment as in FIG. 4C was performed to follow γH2AX and RAD51 IR-induced foci. FIG. 9A shows the accumulation of RPA phosphorylated at S33 under normal and stress conditions are observed in the absence of EYA4. Representative images of foci formation are shown (i; scale bar 10 μm) and quantified (ii; mean±SD; n≥100; **P≤0.01, ***P≤0.001, ****P≤0.0001). Induction is plotted (iii). FIG. 9B shows the time-course of pRPA (S4/S8) following radiation (4Gy) induced DSB formation at T=0 (untreated) and T=1, 2 and 4 hours (4 Gy) are shown for three hairpins that deplete EYA4 (shRNAT-3) and a control (EV) Scale bar 10 μm. Nuclear foci were quantified and plotted as mean±SD; n≥300; **P≤0.01, ***P≤0.001, ****P≤0.0001. FIG. 10A shows the time-course of RAD51 foci formation following radiation (4Gy) at t=0 (untreated), t=1, 2 and 4 hours post irradiation. Scale bar 10 μm. Nuclear foci were quantified and plotted as mean±SD; n≥300; **P≤0.01, ***P≤0.001, ****P≤0.0001. FIG. 10B shows that nuclear RAD51 foci in HeLa Fucci cells were observed without irradiation, or 2 h post 10 Gy-irradiation. Images were acquired by high throughput content imager (Operetta) and analyzed using the GE analysis software.

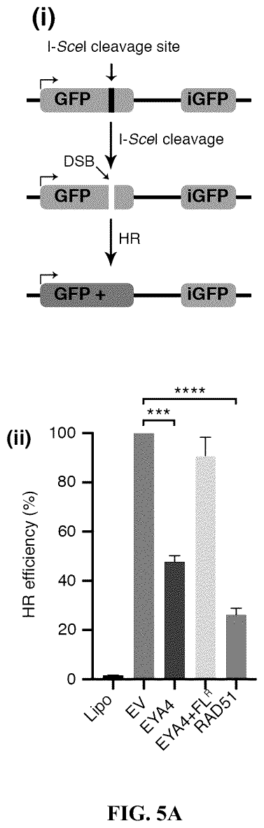

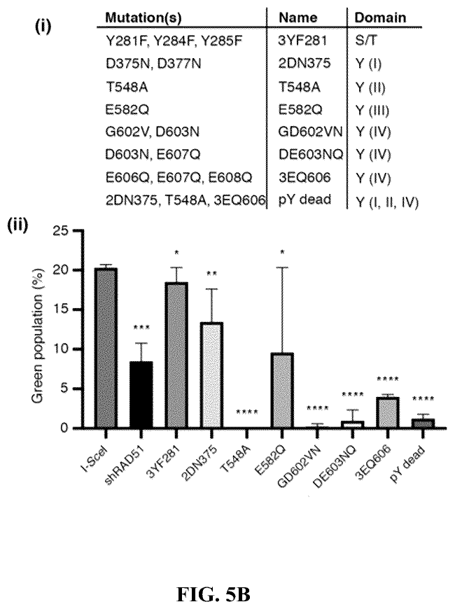

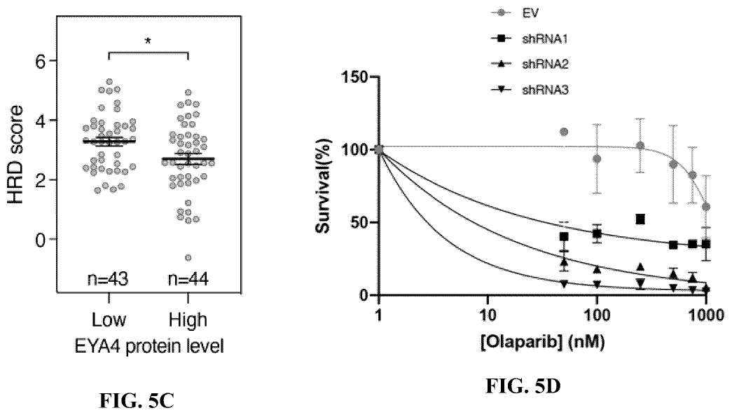

Lack of RAD51 foci indicate that recombination-directed DSB repair might be affected. To ascertain this, the use of the in vitro system developed by the Jasin group (A. J. Pierce, et al., Genes & Development 13, 2633-2638 (1999), K. Nakanishi, et al., Methods in Molecular Biology 745, 283-291 (2011)) was made, which allows direct measurement of HR. EYA4 depleted cells were found less proficient in repairing DSBs by HR (FIG. 5A). Repair was restored to control levels by re-introduction of the full-length EYA4. The EYA4 depleted cells were systematically complemented with various EYA4 constructs containing one or more mutations in the catalytic domain (FIG. 1A). Doing so evidenced that the tyrosine phosphatase domain of EYA4 is essential for HR (FIG. 5B).

Referring to FIG. 5A shows representative data illustrating that the DR-GFP system (i) was used in cells and the percentage of GFP-positive cells was quantified by flow cytometry on >100,000 cells to estimate HR efficiency. shRNA used are indicated. FIG. 5B shows representative data illustrating where mutations were introduced in EYA4 to inactivate phosphatase activity (i). These constructs were used to transfect DR-GFP cells and quantify their individual contribution to HR (ii).

To assess whether reduced EYA4 protein levels might cause HR deficiency more broadly, previously defined HR deficiency scores (G. Peng et al., Nat Commun 5, 3361 (2014)) were determined in 87 CCLE cell lines (Y. Cai et al., Nature 561, 411-415 (2018)). This revealed that cell lines with low EYA4 protein levels show significantly higher HR deficiency compared to cell lines with high EYA4 protein levels (p=0.0167, t test) (FIG. 5C). In accordance with a HR repair defect, cells depleted for EYA4 were also found sensitive to the PARP inhibitor Olaparib in an MTT assay (FIG. 5D).

FIG. 5C shows representative data illustrating 87 cell lines from CCLE. Split in two equal-size groups: EYA4 low and EYA4 high with median EYA4 protein expression level used as cut-off. Means+/−SEM are shown. P value: p=0.0028, t test. HRD score: homologous recombination deficiency score, as defined by Peng et al (G. Peng et al., Nat Commun 5, 3361 (2014)). FIG. 5D shows representative data illustrating a MTT assay was used to measure survival in response to the PARP inhibitor olaparib. For all panels *P<0.05, **P<0.01, ***P<0.001, ****P<0.0001.

### f. RAD51 Y315 is a Target of EYA4

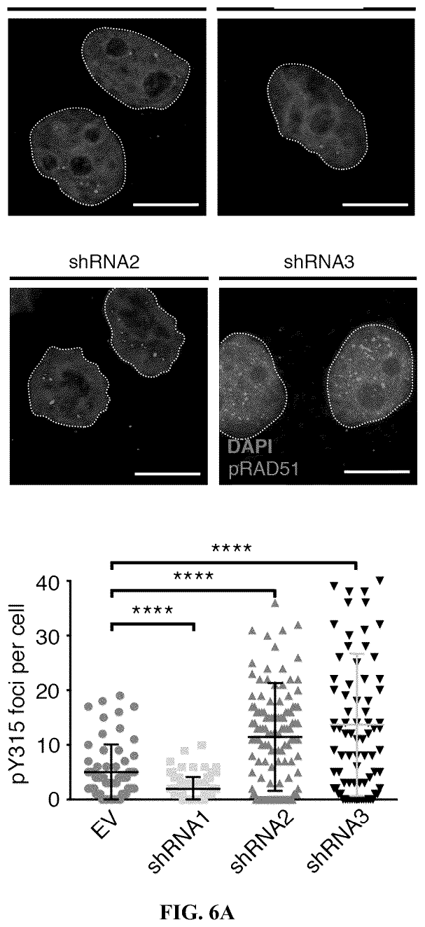

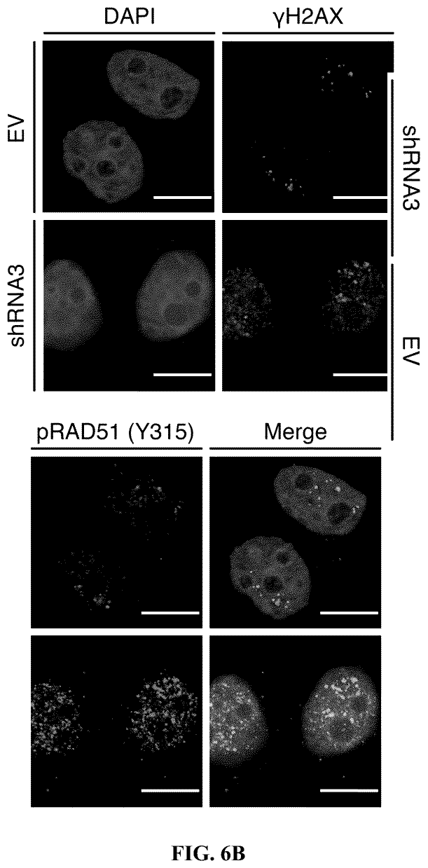

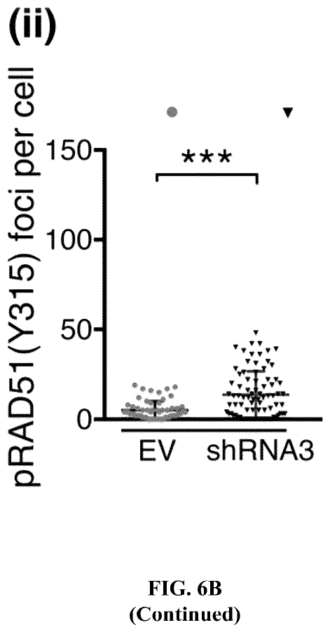

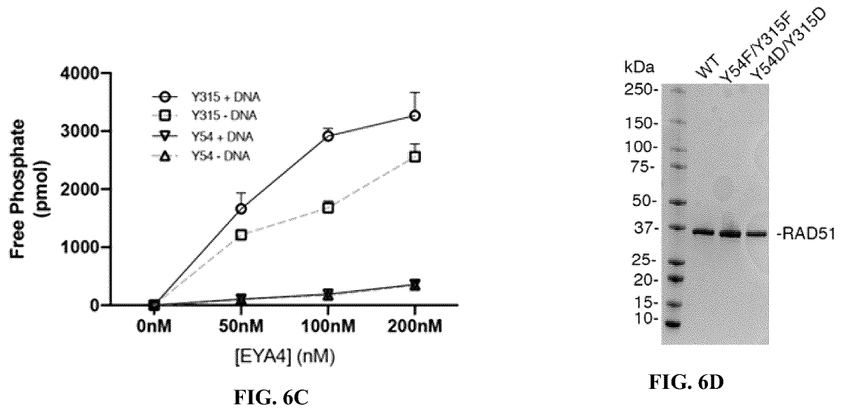

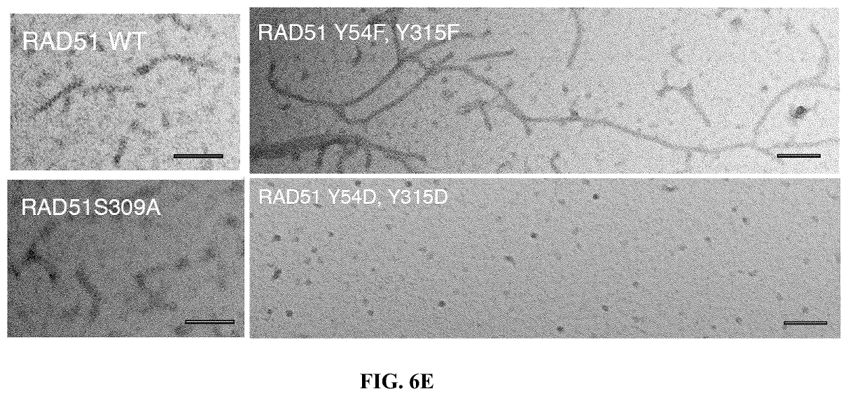

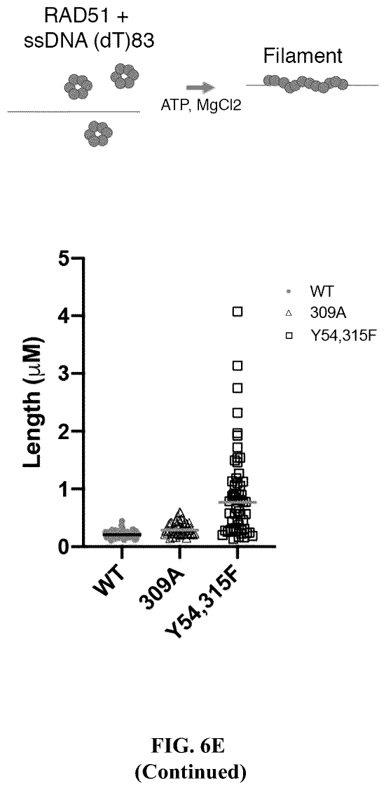

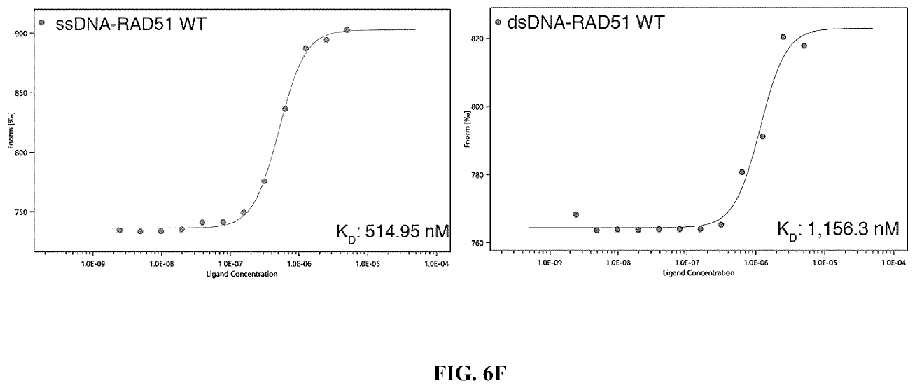

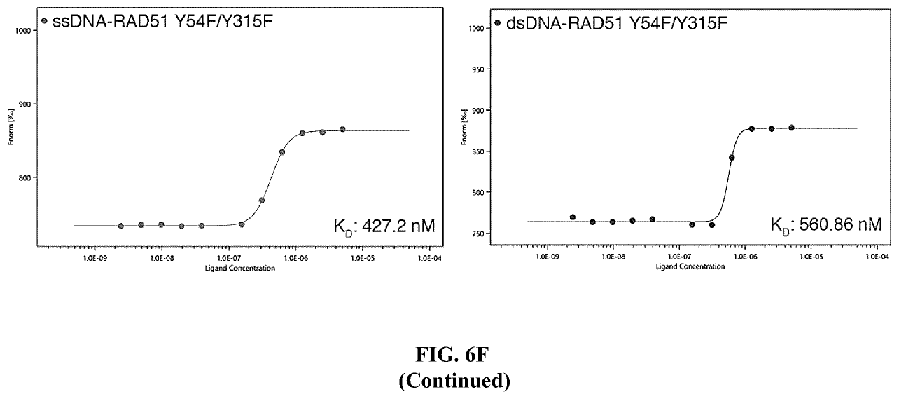

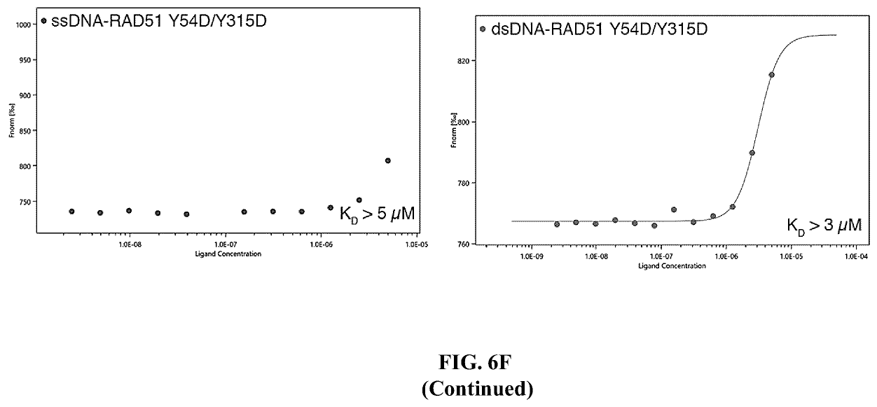

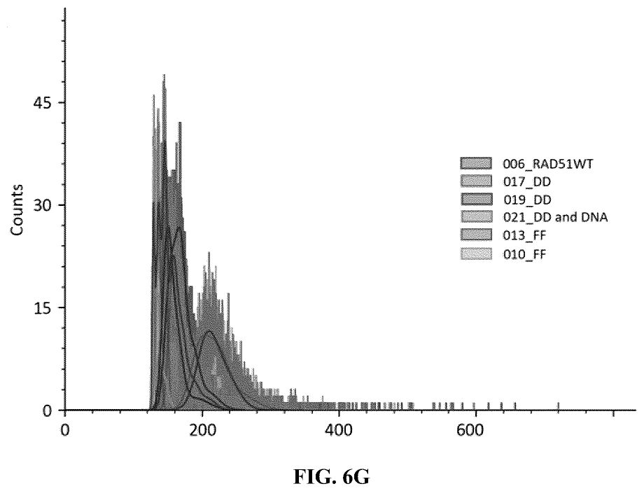

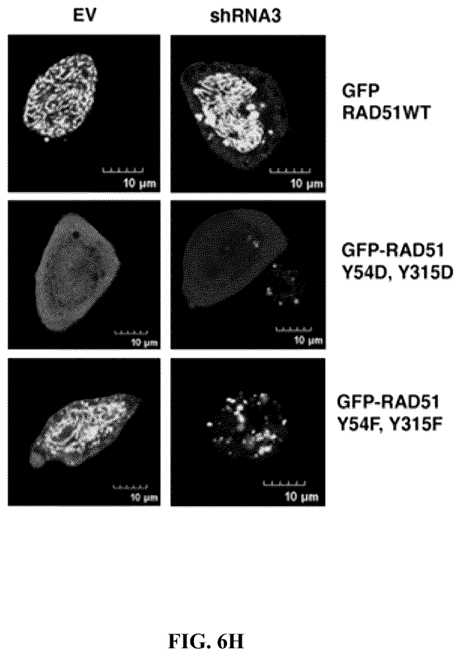

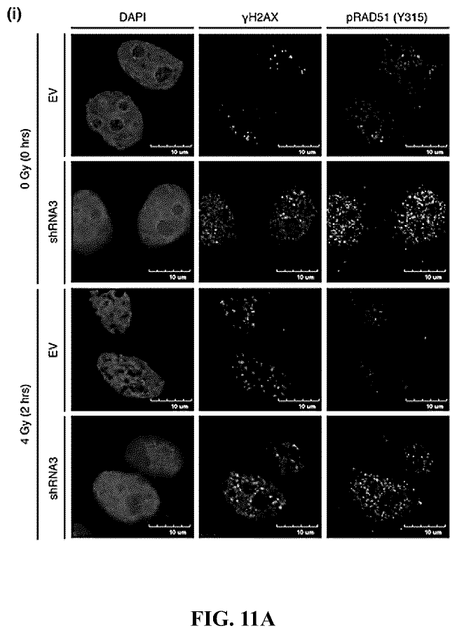

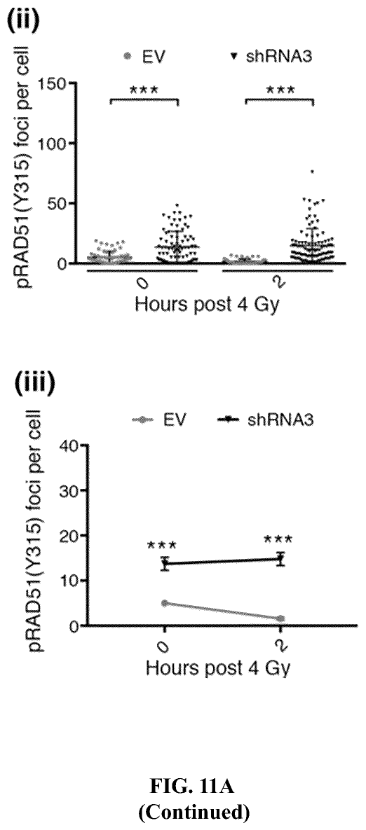

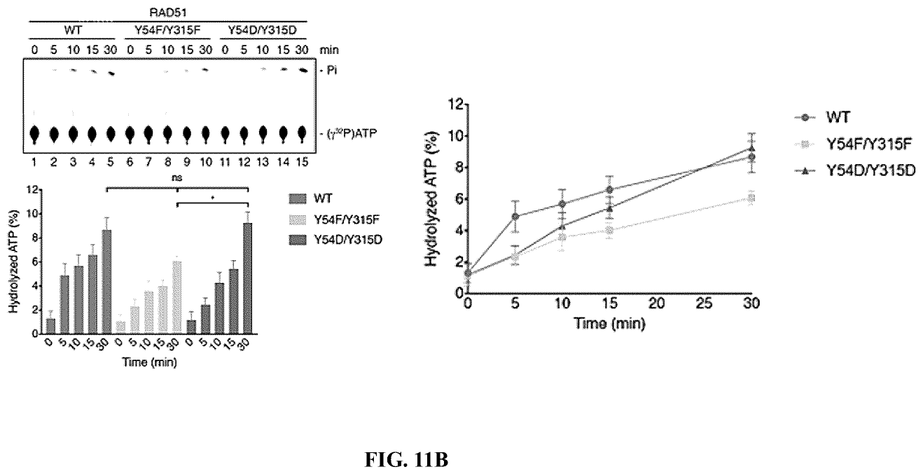

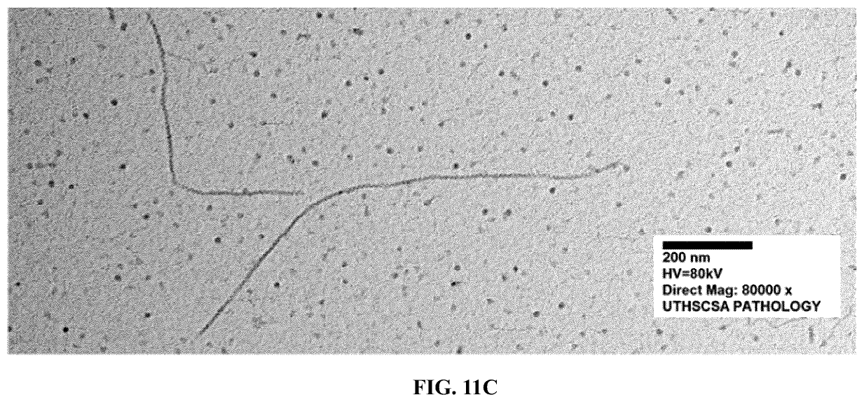

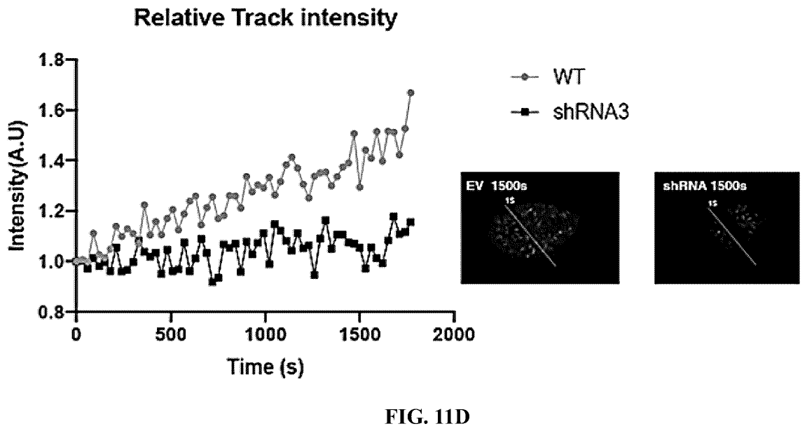

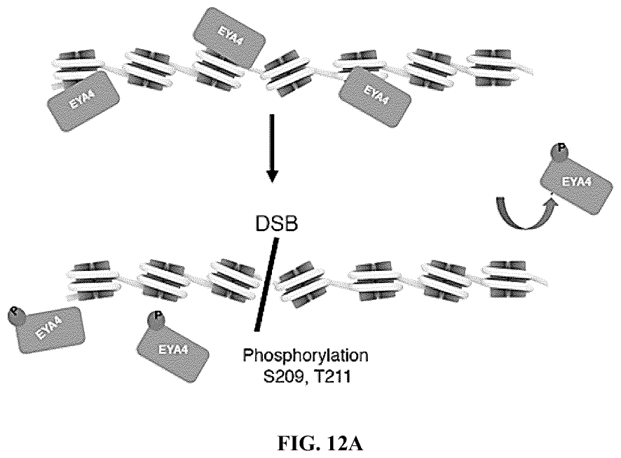

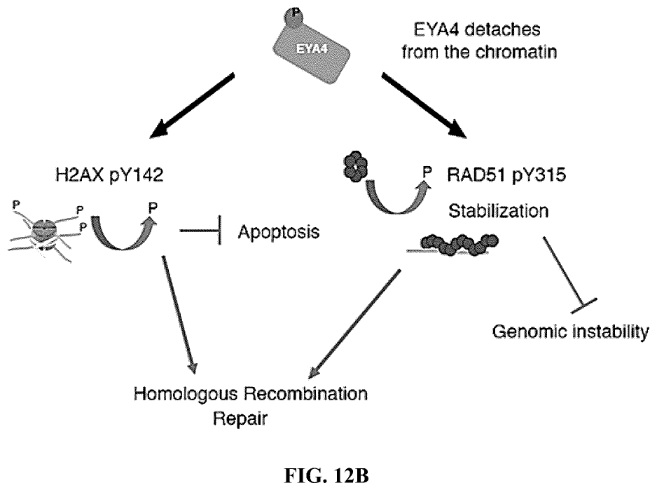

RAD51 phosphorylation levels on residues Y54 and Y315 could influence its function (D. Chowdhury et al., Mol Cell 31, 33-46 (2008), H. Cha et al., Cancer Res 70, 4112-4122 (2010)). The tyrosine kinase c-ABL, or its oncogenic fusion protein BCR/ABL, phosphorylates RAD51 in a sequential manner, on residue Y315 followed by Y54 (M. Popova et al., FEBS Letters 583, 1867-1872 (2009)). This sequential phosphorylation is important for RAD51 recruitment to DSB sites and its strand exchange activity (S. Subramanyam, et al., PNAS 113, E6045-E6054 (2016)). Persistent phosphorylation of Y315, and consequently double-phosphorylated status of RAD51, is known to have an inhibitory effect on HR, due to a defect in nucleofilament formation (B. Alligand, et al., Biochimie 139 (2017)). Using a specific antibody recognizing pY315, it was observed that cells depleted for EYA4 exhibit elevated foci of pY315RAD51, compared to control cells (FIG. 6A). Interestingly, control cells exhibit some pRAD51 in the absence of stress, which decrease in response to IR (FIG. 6B). However, in cells depleted for EYA4, pY315 remains elevated (FIG. 6B, FIG. 11A). Using the malachite green in vitro phosphatase assay, Y315 was identified, but not Y54, to be a substrate for EYA4 (FIG. 6C). Interestingly, the dephosphorylation of RAD51 by EYA4 was again found stimulated by the addition of ssDNA suggesting a common activation mechanism for its phosphatase function. Biochemical characterization of Y54 and Y315 have been performed independently ((M. Popova et al., FEBS Letters 583, 1867-1872 (2009), S. Subramanyam, et al., PNAS 113, E6045-E6054 (2016), B. Alligand, et al., Biochimie 139 (2017)) and very elegant studies from the Spies and Fleury laboratories evidences the role of these residues in RAD51 recombinase activity. However, there are instances where dual phosphorylations can have a very distinct effect than single PTM, as previously exemplified by S139, Y142, in H2AX (J. A. Brown, et al., FEBS Open Bio 2, 313-317 (2012)). Therefore, the combined action of these two residues was investigated. Hence, the double-mutant RAD51 (Y54F, Y315F) that cannot be phosphorylated was purified, as well as the double phospho-mimetic RAD51(Y54D, Y315D), which mimics a phosphorylated tyrosine that cannot be dephosphorylated (FIG. 6D). Based on the cell-based data and the literature available on these RAD51 tyrosine residues, the ability of RAD51(Y54F, Y315F) to polymerize onto ssDNA and form nucleoprotein filaments was investigated. Surprisingly, this mutant was observed by electron microscopy and negative staining (FIG. 6E) to be much more proficient at polymerizing than the WT protein (FIG. 6E). It can form filaments across ˜10 lengths of ssDNA (83dT) in the mutant (FIG. 6E), while WT RAD51 coats ssDNA as individual filaments, without any tethering activity (FIG. 6E). In light of these surprising data, a T309A mutant that is less efficient in HR (P. B. Narayanaswamy, et al., Cell Death Dis 7, e2383 (2016)) but does not affect filament formation was purified and used. This mutant did not exhibit significant differences when compared to WT RAD51. The RAD51 (Y54D, Y315D) phosphomimetic formed globular structures that are reminiscent of the hexameric ring. Minimum variations in the ATPase activity were observed between RAD51 WT and mutants (FIG. 11B), that cannot fully explain the phenotype observed. DNA binding experiments by microscale thermophoresis (MST) were conducted (FIG. 6F). Surprisingly, it was found that Y54F, Y315F binds to ssDNA with an affinity comparable to the WT RAD51. However, it is much more avid for dsDNA. In this experiment, the hill coefficient was very high at 7.07 (Table 5 and FIG. 11C), which is consistent with cooperative binding, phase separation and/or condensation. These data indicate a different DNA binding modality for the Y54F, Y315F mutant, possibly by aggregation onto the DNA. Even more surprisingly, the phospho-mimetic Y54D, Y315D was found to bind neither ssDNA nor dsDNA (FIG. 6F) with a good affinity. While this was consistent with our EM observations, it could not rule out that this phospho-mimetic can bind to nucleic acid. Indeed, in two independent protein preparations (no added DNA and no ATP nor MgCl₂ in the storage buffer), rare, long filament structures were observed (FIG. 11C). Whether RAD51 (Y54D, Y315D) co-purifies with specific sequences of DNA, RNA, or forms spontaneous filamentous polymers on its own remains to be elucidated. Mass Photometry analysis (Reyfen) confirmed that the WT RAD51 forms hexameric rings in solution at the expected −200 kDa mark, the Y54D, Y315D and Y54F, Y315F readings indicated possibly different configurations (FIG. 6G). When introducing GFP-fused WT; Y54F, Y315F or Y54D, Y315D constructs in cells either expressing EYA4 (EV) or not (shRNA3), it was observed that the WT forms structures as previously described for GFP-RAD51 (M. Reuter et al., J Cell Biol 207, 599-613 (2014)) in the EV cells. However, the Y54D, Y315D is found non-nuclear, consistent with its inability to bind to DNA (FIG. 6H). In cells transfected with shRNA3, both the WT and the phospho-mutant constructs form condensates that are consistent with the MST data, namely that dephosphorylated RAD51 is prone to phase separation on DNA. It had been previously observed that the overexpression of EYA4-WT in cells led to an increase of RAD51 staining in otherwise untreated cells (see FIG. 4B). Since EYA4 overexpression leads to accumulation of RAD51 in vivo (FIG. 4) and dephosphorylates RAD51, the question remained whether RAD51 (Y54F, Y315F) might be more stable than RAD51 WT or RAD51 (Y54D, Y315D). Cells expressing GFP-RAD51 WT were subjected to photo bleaching experiments. Analysis of fluorescence intensity overtime after photodamage showed that RAD51 WT mobility is diminished in cells depleted for EYA4 when compared to control cells expressing EYA4 (FIG. 11D).

Referring to FIG. 6A shows representative data illustrating phosphorylation at residue Y315 can be observed in cells using a specific antibody. FIG. 6B shows representative data illustrating the pY315 levels in cell respond to irradiation. FIG. 6C shows representative data illustrating EYA4 dephosphorylates residue pY315 in a malachite green assay and the activity is stimulated by DNA. FIG. 6D shows representative data illustrating RAD51 WT, phospho-mutant Y54F, Y315F and phospho-mimic Y54D, Y315D were purified near homogeneity. FIG. 6E shows representative data illustrating representative electron microscopy micrographs of the RAD51 proteins on DNA. FIG. 6F shows representative data illustrating microscale thermophoresis measurements of RAD51 proteins on ssDNA or dsDNA and the affinity constants. FIG. 6G shows representative data illustrating mass photometer measurement of apparent sizes for RAD51 polymers in suspension. FIG. 6H shows representative data illustrating that introduction of RAD51 proteins as GFP-constructs in HeLa cells allows live imaging and the tracking of RAD51 structures formation and their subcellular localization. FIG. 11A shows pY315RAD51 form dynamic foci that decrease in response to irradiation. Top panel (OGy) is depicted in the main figures, bottom panel shows response to irradiation (2 h post 4Gy). FIG. 11B shows ATPase activity was measured by thin layer chromatography in the presence of 32P labelled ATP and hydrolyzed ATP was quantified over time and plotted. FIG. 11C shows a fraction of the RAD51 Y54D, Y315D mutant protein preparation forms long pre-synaptic filament-like structures without added nucleic acid. FIG. 11D shows WT-GFP-RAD51 transfected in control or EYA4 depleted (shRNA3) cells shows variation of mobility over time following laser (405) micro ablation. GFP intensity of the track was measured using Olympus CellSens and relative intensity to t0 (prior laser) is plotted.

TABLE 5

| | ssDNA | ssDNA | ssDNA | dsDNA | dsDNA | dsDNA |
|---|---|---|---|---|---|---|
| Parameter | WT | YF, YF | YD, YD | WT | YF, YF | YD, YD |
| Unbound | 736.31 | 734.29 | NA | 764.48 | 763.98 | 767.4 |
| Bound | 902.77 | 863.69 | NA | 822.95 | 877.97 | 828.49 |
| EC₅₀ | 514.95 nM | 427.2 nM | > 5000 nM | 1156.3 nM | 560.86 nM | 3.0719 μM |
| EC₅₀ | ±18.1 nM | ±7.3 nM | NA | ±162.5 nM | ±11.7 nM | ±0.4951 μM |
| Confidence | | | | | | |
| Hill Coeff. | 2.662 | 3.312 | NA | 2.2959 | 7.07 | 2.6695 |
| Signal to | 59.0 | 95.2 | NA | 18.0 | 37.9 | 45.7 |
| Noise | | | | | | |
| Resp. | 166.4557 | 129.4027 | NA | 58.474 | 113.98 | 61.0865 |
| Amplitude | | | | | | |

Without wishing to be bound by theory, the combination of this increase in stability coupled to a high avidity for dsDNA and a slightly decreased ATPase activity of RAD51 (Y54F, Y315F) could explain RAD51 strong staining in cells overexpressing EYA4.

Taken together, the data demonstrate that EYA4 promotes DNA repair by homologous recombination. It is phosphorylated upon DNA damage, dephosphorylates histone H2AX to promote repair over apoptosis, and contributes to HR repair through the dephosphorylation and subsequent stabilization of RAD51.

### g. The Timing of Repair Events is Affected in EYA4 Depleted Cells

EYA4 depleted cells exhibit elevated levels of pY412-H2A as shown by western blotting and immunofluorescence. When cells were subjected to irradiation (10Gy for western blots or 4Gy for foci formation), minimal variations were observed over time by western blotting (FIG. 7A). Without wishing to be bound by theory, this could be explained by the low abundance of the protein modification, which has been described previously as rare in proteomics studies, and the difficulty to protect phospho-tyrosines during protein extraction. In cells, the accumulation of H2AX-pY142 was more evident and we observed elevated pY142 in the cells depleted for EYA4 when compared to the controls. In addition, pY142 remains elevated at 4 h post irradiation in EYA4 depleted cells and decrease in the controls from 2 h. At 20 h post irradiation, the shRNA treated cells only exhibit large clusters of pY142 stain (FIG. 7B).

### h. EYA4 Mapping of the DNA Binding Domain

Various constructs were used to investigate the DNA binding activity of EYA4. EYA4 is a very difficult protein to purify, as it is largely disorganized from residues 1 to 320. EYA4 is prone to aggregation and phase separation, and for these studies, it could not be expressed or purified from E. coli despite trying many constructs. Using new generation fluorescent tags that are super folder and can help solubilize proteins in vivo, it was possible to express and purify GFP or m-cherry (FIG. 8A) in human cell suspensions and Sf9 insect cells, respectively. Full length EYA4, as well as the N-terminal (1-365) and the C-terminal (358-639) halves of the protein, were expressed and purified (FIG. 8A). EMSA was conducted using these three constructs and it was observed that EYA4 full length binds ssDNA with a high affinity, but in the range of concentration used (10-500 nM; FIG. 8B) it exhibits little activity in dsDNA binding. Using the two halves of EYA4, it was verified that the N-terminus contains the full activity (FIG. 8C) while the C-terminus portion does not contain any DNA binding activity. As 1-365 is easier to purify and concentrate, and, thus, more suited for experiments such as MST than the full length WT EYA4, subsequent DNA binding activity investigations were performed using 1-365. Interestingly, the full length EYA4 containing the SQTQ mutations S209D, S21 ID is extremely well expressed, can be purified in large amounts, and can be easily concentrated.

### i. Discussion

Phosphorylations are frequent post-translational modifications on proteins and they play key roles in a wide range of essential cellular functions. This includes regulation of DNA damage repair pathways, as kinases trigger cascades of protein activation and orchestrate their timely recruitment to DNA breaks and adducts. Most phosphorylation events in DNA repair that are well characterized and now utilized as readout of repair progression and efficiency, involve serine and threonine residues. Here, it is described that tyrosine residues on H2AX and RAD51, which have been described before as phosphorylated by the c-Abl kinase, are favored substrates of the protein phosphatase EYA4. Like the kinase DNA-PK, the activity of EYA4 is greatly stimulated by DNA binding. A fraction of EYA4 is nuclear, and it locates at the chromatin in dividing cells, through its N-terminal domain, which we here show directly interacts with histones H2A and H2B, as well as with DNA. Following DNA damage, EYA4 dephosphorylates pY142 on H2A, detaches from the chromatin likely after being phosphorylated by ATM, and dephosphorylates RAD51 to promote HR.

In recent years, it has become clear that RAD51 plays an important role in DNA metabolism, beyond vegetative double strand break repair and its function is not always mediated by BRCA2 alone (F. Prado, Genes (Basel) 12 (2021); M. J. Cabello-Lobato et al., Cell Rep 36, 109440 (2021); M. Tarsounas, D. Davies, S. C. West, Oncogene 22, 1115-1123 (2003)) or even BRCA-dependent. Here, it is shown that dephosphorylated RAD51 is more stable than its phosphorylated version, in cells and in vitro, and more proficient to nucleate onto ssDNA. It exhibits DNA binding modalities that diverge strongly from the WT protein, and was found to accumulate rapidly onto dsDNA. Whether this promotes duplex capture for homologous recombination or targets different DNA structures warrants further studies, including a biophysical characterization of the nucleoprotein filaments it can assemble. RAD51 is often upregulated in p53 deficient cancers. It is known to be overexpressed in breast, cervical, ovarian, pancreatic and other cancers (T. Takenaka et al., CInt J Cancer 121, 895-900 (2007); H. Maacke et al., Int J Cancer 88, 907-913 (2000)), where it causes drug resistance (H. L. Klein, DNA Repair (Amst) 7, 686-693 (2008); Y. Feng, et. al., Cancer Cell Int 21, 249 (2021); M. M. Hoppe et al., EMBO Mol Med 13, e13366 (2021)). Lack of phosphorylation by cAbl or excessive dephosphorylation of RAD51 by EYA4 could both lead to its accumulation and pathogenicity. The exact mechanism by which EYA4 undergoes post-translational modifications in response to DNA damage and how it regulates its interaction with other DNA repair proteins remains to be investigated. However, overall, these data start explaining the link between EYA4 and carcinogenesis. Decreased EYA4 levels in breast cancer samples might help identify HR deficiency, while overexpressed or hyperactive tyrosine phosphatases could predict RAD51 stabilization, accumulation, and resulting drug resistance. Targeting EYA4 could be of interest for the future development of novel cancer therapies, especially these aimed at decreasing uncontrolled DNA damage repair in drug resistant tumors.

### 3. EYA4 Inhibition Afford Broad Spectrum Utility in Cancer Treatment

As detailed herein above, the atypical protein phosphatase EYA4 plays a central role in regulating and coordinating DNA damage repair pathways. By promoting faithful double strand break repair through dephosphorylation of RAD51 and H2AX, and limiting non-homologous end joining, by dephosphorylating 53BP1 EYA4 is a protector of genomic stability and integrity. It limits gross chromosome rearrangements, chromosome fusions, and steers cells toward repair pathways rather than apoptosis following DNA damage.

DNA repair is tightly regulated and accumulation of DNA repair proteins is almost always as deleterious as their depletion or mutation. Increase in either homologous recombination or DNA end joining can lead to illegitimate recombination, break point fusions, and severe genomic rearrangements. Overexpression of 53BP1 and RAD51 for instance, are linked to aggressive cancers and drug resistance.

EYA4 is often found overexpressed in breast cancer and in esophageal cancer, and its accumulation can destabilize the genome by promoting homologous recombination even in G1 phase of the cell cycle, when it should be avoided at all cost and end joining is to be the dominant repair mechanism. EYA4 could thus be an ideal target for drug development, and its inactivation could be used to treat the many tumors overexpressing it. Here, the synthesis and characterization of a novel class of molecules that inhibit specifically and efficiently the tyrosine phosphatase activity of EYA4 is described. It is found that blocking the tyrosine phosphatase activity of EYA4 recapitulates major phenotypes observed in cells depleted for EYA4. Chemical blockade of the tyrosine dephosphorylation activity results in the accumulation of phosphorylated RAD51, which is less stable than its dephosphorylated counterpart and less prone to hyper-recombination. It is also found that DNA damage repair function by homologous recombination is lessened by increasing doses of the inhibitor, and higher doses of EYA4 inhibitor kills cancer cells of various subtypes by promoting apoptosis. Taken together, these results evidence that EYA4 could be efficiently targeted in a broad range of cancers. These include tumors that present excessive DNA repair capabilities acquired by secondary mutations in the course of chemotherapy, as well as tumors overexpressing RAD51 and other DDR proteins. In addition, tumor growth and metastasis could be tamed or even fully suppressed by EYA4 inhibition, in cancers known to overexpress EYA4 such as esophageal and triple negative breast cancers.

### a. Materials and Methods

### i. Chemical Synthesis

All operations were carried out at room or ambient temperature, that is, in the range of 18-25° C.; evaporation of solvent was carried out using a rotary evaporator under reduced pressure with a bath of up to 50° C.; reactions were monitored by thin layer chromatography (tlc) and reaction times are given for illustration only. Unless otherwise indicated all reactions were conducted in standard commercially available glassware using standard synthetic chemistry methods and setup. All air- and moisture-sensitive reactions were performed under nitrogen atmosphere with dried solvents and glassware under anhydrous conditions. Starting materials and reagents were commercial compounds of the highest purity available and were used without purification (See list of specific reagents below). Solvents used for reactions were indicated as of commercial dry or extra-dry or analytical grade. Analytical thin layer chromatography was performed on aluminum plates coated with Merck Kieselgel 60F254 and visualized by UV irradiation (254 nm) or by staining with a solution of potassium permanganate. Flash column chromatography was performed on Biotage Isolera One 2.2 using commercial columns that were pre-packed with Merck Kieselgel 60 (230-400 mesh) silica gel. Final compounds for biological testing are all >95% purity as determined by HPLC-MS and ¹H NMR. ¹H NMR experiments were recorded on Agilent DD2 400 MHz spectrometers at ambient temperature. Samples were dissolved and prepared in deuterated solvents (CDCl₃, CD₃OD and DMSOd₆) with residual solvents being used as the internal standard in all cases. All deuterated solvent peaks were corrected to the standard chemical shifts (CDCl₃, dH=7.26 ppm; CD₃OD, dH=3.31 ppm; DMSOd₆, dH=2.50 ppm). Spectra were all manually integrated after automatic baseline correction. Chemical shifts (d) are given in parts per million (ppm), and coupling constants (J) are given in Hertz (Hz). The proton spectra are reported as follows: d (multiplicity, coupling constant J, number of protons). The following abbreviations were used to explain the multiplicities: app=apparent, b=broad, d=doublet, dd=doublet of doublets, ddd=doublet of doublet of doublets, dddd=doublet of doublet of doublet of doublets, m=multiplet, s=singlet, t=triplet. All samples were analyzed on Agilent 1290 series HPLC system comprised of binary pumps, degasser and UV detector, equipped with an auto-sampler that is coupled with Agilent 6150 mass spectrometer. Purity was determined via UV detection with a bandwidth of 170 nm in the range from 230-400 nm. The general LC parameters were as follows: Column—Zorbax Eclipse Plus C18, size 2.1×50 mm; Solvent A: 0.10% formic acid in water, Solvent B: 0.00% formic acid in acetonitrile; Flow rate—0.7 mL/min; Gradient: 5% B to 95% B in 5 min and hold at 95% B for 2 min; UV detector—channel 1=254 nm, channel 2=254 nm. Mass detector Agilent Jet Stream—Electron Ionization (AJS-ES).

### ii. FUCCI Analysis by Live Imaging

Experimental set up: Red and Green U2932 cells were synchronized using a double thymidine block prior to the start of this experiment. After release from the last thymidine block, the cells were allowed to rest in fresh media for 5 hours to prevent synchronization related cell death. Following this rest period, approximately 10-50 cells were added to a 96 well plate that was pretreated with Poly-L-Ornithine (increases cell adhesion to plate, keeping cells in frame for the experiment). The cells were then treated with alisertib, Aurkin A, EYA4, or combination therapy with a final volume of 1% DMSO (in 100 uL well-RPMI 1640 with 10% FBS). Each drug condition had a 3× replicate. Plates were analyzed via an Incucyte SX5 plate reader, scanning at 20× in both red and green fluorescence every 30 minutes.

Analysis: Cell by cell analysis was preformed via the Incucyte software. Red and Green fluorescent gating was preformed based on the control image time course. The average fluorescent (colorless G0, red G1, green S-G2-M, yellow G1/S transition) cell counts for each experimental group (each data point is an average of 3× replicates). The number of red, green, and yellow fluorescent cells was added together to determine the total cell fluorescent count for each experimental group. Each color was then divided by the total fluorescent cell count to get a percentage fluorescence (raw data with formals found in the excel spread sheets attached). This was the Red and Green conditions were then graphed in Prism (data file with graphs attached). Colorless cells (late M/G0/senescent) population was excluded from the analysis.

### b. Synthesis of Molecules and Description of the Libraries

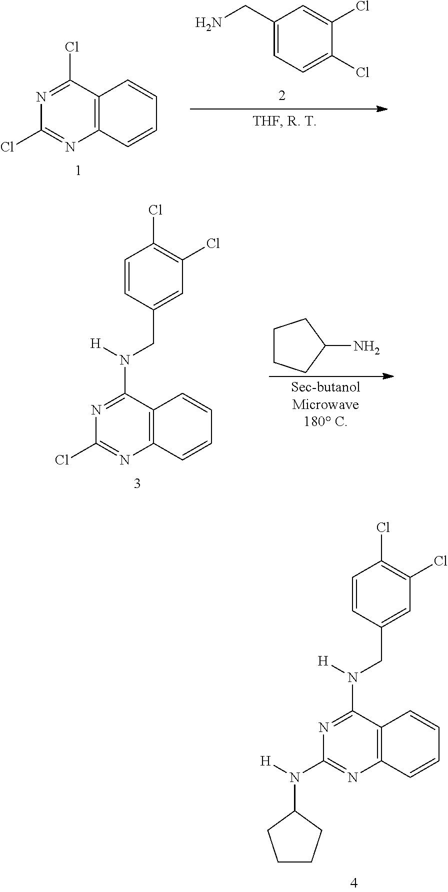 [SMILES: Clc1nc(Cl)c2ccccc2n1 ; NCc1ccc(Cl)c(Cl)c1 ; Clc1nc(NCc2ccc(Cl)c(Cl)c2)c2ccccc2n1 ; NC1CCCC1 ; Clc1ccc(CNc2nc(NC3CCCC3)nc3ccccc23)cc1Cl]

Libraries of compounds were synthesized using the procedures detailed elsewhere herein. For compounds 3 and 4 used in this study, the specific steps were followed: 2-chloro-N-(3,4-dichlorobenzyl)quinazolin-4-amine (3): To a stirring solution of 2,4-dichloroquinazoline 1 (5.0 g, 25.1 mmol) in THF (125 mL) at room temperature was added 3,4-dichlorobenzylamine 2 (4.0 mL, 30.3 mmol). The mixture was stirred for 24 hr at room temperature, during which time a precipitate formed. The slurry was filtered and washed with hexanes. The collected filtrate was slurried with DCM/hexanes and filtered. The filtrate was washed with hexanes, collected and dried under reduce vacuum to yield 4.4 g (52% yield) of 2-chloro-N-(3,4-dichlorobenzyl) quinazolin-4-amine (3) as a white powder.

For the N2-cyclopentyl-N4-(3,4-dichlorobenzyl) quinazoline-2,4-diamine (4, CIDD-0149689), synthesis was performed using the following procedure: To a 30 mL microwave reaction vial was added 2-chloro-N-(3,4-dichlorobenzyl)quinazolin-4-amine 3 (1.0 g, 2.95 mmol), sec-butanol (15 mL) and cyclopentylamine (251 mg, 2.95 mmol). The mixture was irradiated in an Anton Par microwave reactor for 30 minutes at 180° C. The reaction was cooled to room temperature, during which time a precipitate formed. The solids were filtered, washed with hexanes and dried under reduced pressure to yield N₂-cyclopentyl-N4-(3,4-dichlorobenzyl) quinazoline-2,4-diamine (970 mg, 85% yield) as a white powder.

### c. Discussion

EYAT-3 proteins have been described to dephosphorylate residue Y142 on the histone variant H2AX and the same activity was recently confirmed in EYA4. Using the synthetic peptide KATQASQE{pTyr} as substrate and a commercial colorimetric malachite green assay (Sigma) that allows to quantify ATP released in the buffer by a phosphatase, EYA4's tyrosine phosphatase activity was assayed in the presence of putative inhibitors.

In a malachite green assay, the tyrosine but not serine/threonine phosphatase activity was inhibited by CIDD molecule—149689. For this assay, full length EYA4 purified near homogeneity as previously described was incubated with increasing amounts of inhibitor, then mixed with either peptide KATQASQE{pTyr} from H2AX or with peptide from RAD51. In both cases, the phosphatase activity was significantly reduced. Interestingly, when a large polypeptide encompassing the first 365 residues of EYA4 was incubated, and which contains the serine/threonine phosphatase domain but not the tyrosine domain, with phosphatase inhibitors in the presence of a 53BP1-derived peptide, the phosphatase activity was not inhibited.

Both the serine/threonine and the tyrosine phosphatase domains contain similar catalytic residues, mostly acidic but the tyrosine inhibitor was found specific.

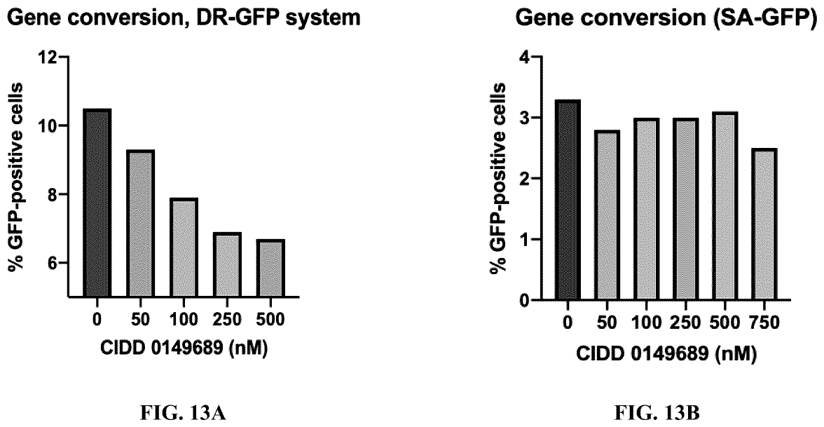

Referring to FIG. 13A and FIG. 13B, EYA4 tyrosine phosphatase inhibitor recapitulates the HR deficient phenotype but does not affect other DNA damage repair pathways.

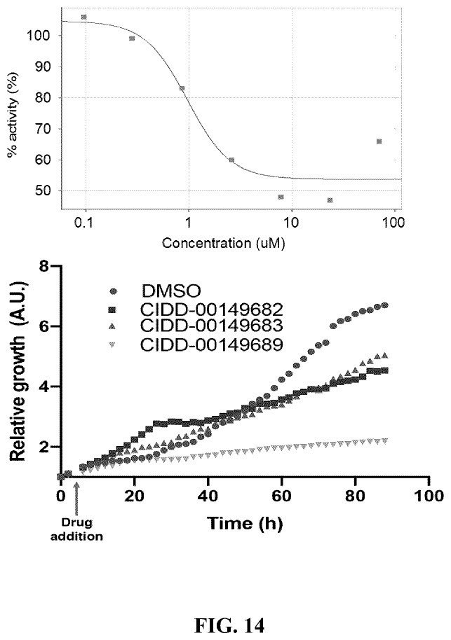

Referring to FIG. 14, an exemplary compound efficiently inhibits tyrosine phosphatase in the micromolar concentration range, and efficiently suppress cell growth in cells expressing high levels of EYA4.

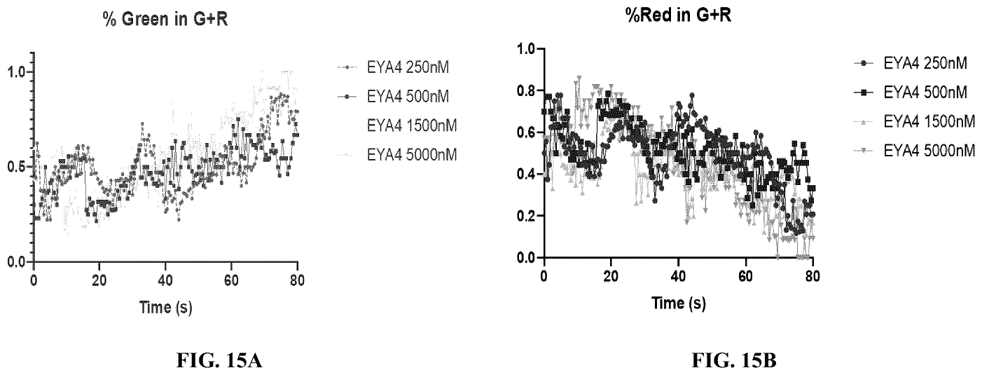

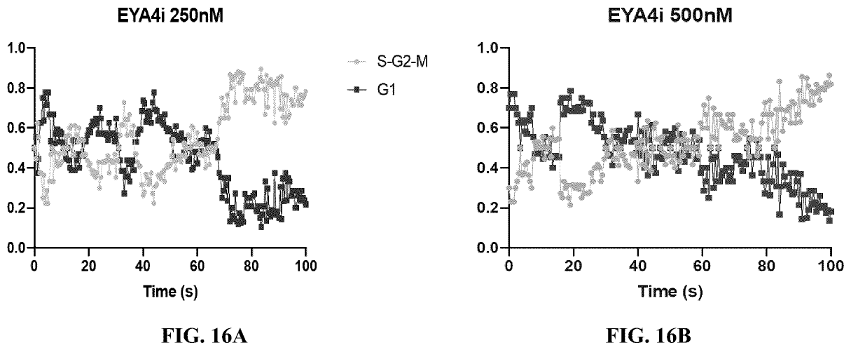

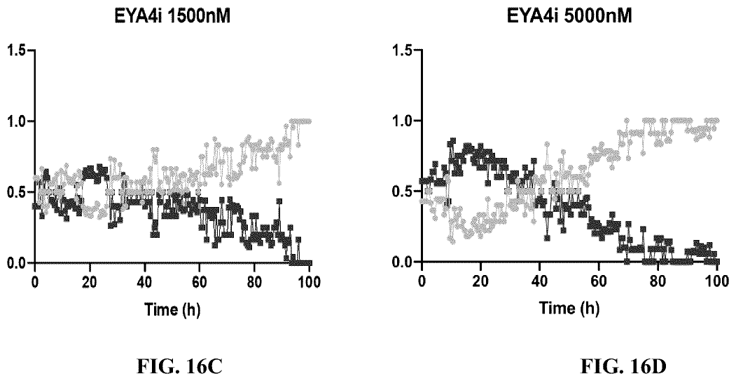

Referring to FIG. 15A, FIG. 15B, and FIG. 16A-D, EYA4i is synthetic lethal with kinase inhibitors in breast and leukemia cell lines.

### 4. EYA4 Drives Breast Cancer Progression and Metastasis Through its Novel Role in Replication Stress Avoidance

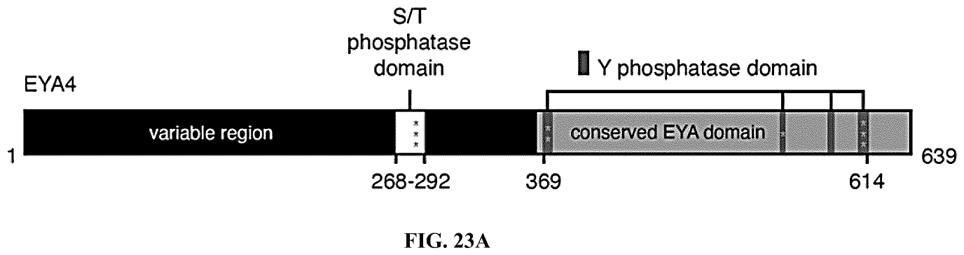

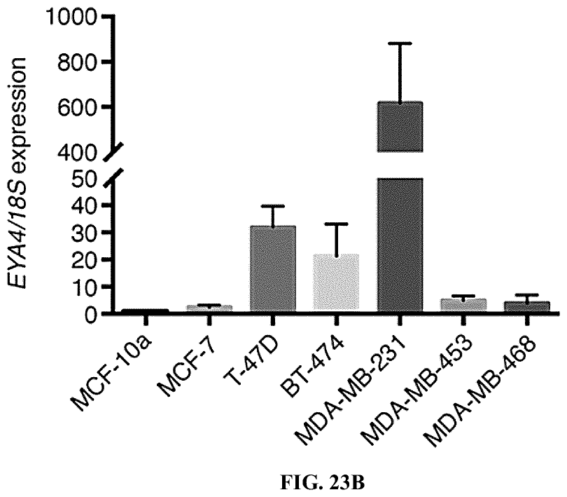

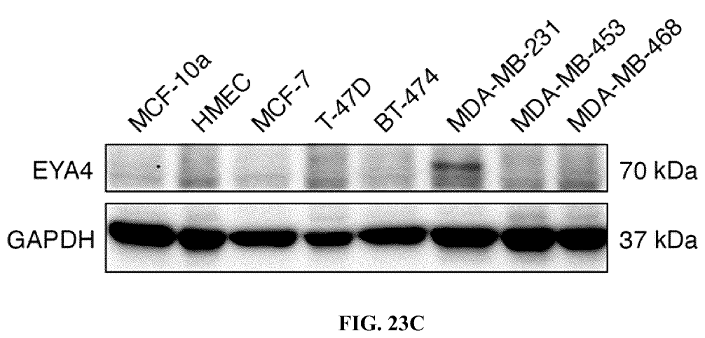

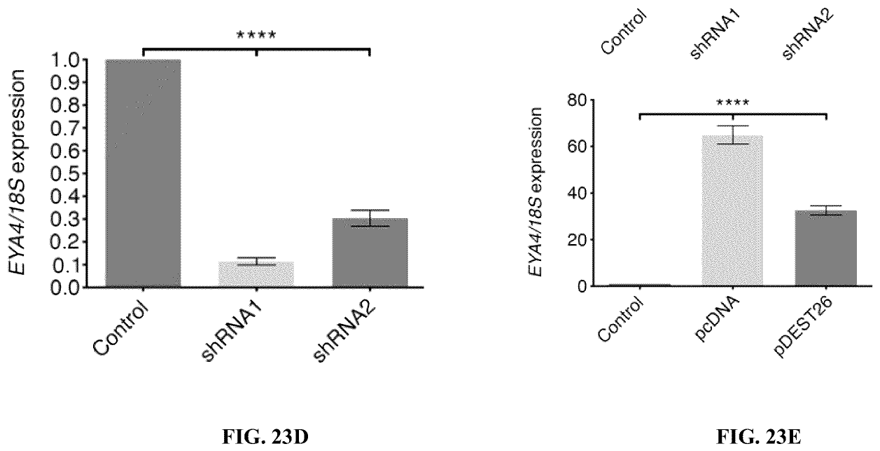

The Eyes Absent family (EYA1-4) is a unique group of dual-functioning protein phosphatases, which have been shown to promote cell proliferation, invasion, migration, and survival in a variety of cancers (Kong D, et al. (2019) American Journal of Translational Research 11(4): 2328-38; Xu H, J et al. (2019) Frontiers in Oncology 9(26); Zhu J, et al. (2021) World Neurosurgery 149: e1174-e9). Members of the EYA family possess N-terminal transcriptional co-activation and threonine phosphatase activity, and C-terminal tyrosine phosphatase activity (Tootle T L, et al. (2003) Nature 426: 299-302; Okabe Y, et al. (2009) Nature 460: 520-4; Rebay I. (2016) Molecular and Cellular Biology 36(5): 668-77). The highly conserved C-terminal domain, also known as the EYA domain (ED), contains a haloacid dehalogenase (HAD) signature sequence, making them the only known HAD-family tyrosine phosphatases (FIG. 23A) (Tootle T L, et al. (2003) Nature 426: 299-302). As the founding members of a new class of non-thiol-based protein tyrosine phosphatases, EYAs have a unique active site, using aspartic acid rather than cysteine as the nucleophile, making these atypical phosphatases attractive targets for specific inhibition with small molecules. However, the biological functions and cellular targets of these dual-phosphatases remain largely unknown, particularly for EYA4.

Defects in EYA4 have been linked to different developmental disorders including hearing loss (Morin et al. (2020) Scientific Reports 6213) and cardiomyopathy (Ahamadmehrabi et al. (2021) Human Genetics 140: 957-67). EYA4 has also been associated with cancer in various organs. In malignant peripheral nerve sheath tumors (MPNST) EYA4 is over-expressed (Miller et al. (2010) Oncogene 29(3): 368-79), whilst it is down-regulated in esophageal adenocarcinoma (Zou et al. (2005) Cancer Epidemiology, Biomarkers and Prevention 14(4): 830-4; Luo et al. (2018) Cancer Science 109(6): 1811-24), hepatocellular carcinoma (Hou et al. (2014) Annals of Surgical Oncology 21(12): 3891-9), lung cancer (Wilson et al. (2014) Oncogene 33(36): 4464-73) and colorectal cancer (Kim et al. (2015) Molecular Carcinogenesis 54(12): 1748-57), where the EYA4 gene promoter has been found to be hypermethylated. Consistent with this, our group and collaborators identified EYA4 as a potential novel breast cancer gene (Stirzaker (2015) et al. Nature Communications 6: 5899). Specifically, the observation that EYA4 is hypermethylated in the first intron-exon junction particularly in triple-negative breast cancer patients when compared to matched normal samples prompted the study of its role in carcinogenesis and its cellular functions. EYA4 was inactivated or overexpressed in a variety of cell lines and investigated the resulting phenotypes, including cell cycle progression and DNA replication efficiency.

Over-expression of EYA4 was shown to increase proliferation and migration in breast cancer cells, features that are linked with aggressive breast cancer in vivo. The function of EYA4 in promoting breast cancer growth and metastasis is also supported by in vivo xenograft studies showing that silencing of EYA4 expression in MDA-MB-231 cells leads to reduced cancer burden and distant metastasis. The serine/threonine phosphatase activity of EYA4 was found to be essential for breast cancer progression and metastasis, but not its tyrosine phosphatase. In cells, EYA4 depletion was revealed to promote endoreplication and consequently polyploidy, a phenomenon that can occur in response to stress (Lang L, et al. Science Direct. 2020; 54:85-92; Matsuda M, et al. Plant Cell Reports. 2018; 37:913-21) and can cause drug resistance (Shu Z, et al. Trends in Cell Biology. 2018; 28(6):465-74). The absence of EYA4 leads to spontaneous replication stress characterized by activation of key cell cycle checkpoints (pChk1 and pChk2), sensitivity to hydroxyurea, and accumulation of endogenous DNA damage, as indicated by increased γH2AX levels. Upon induction of replication stress by hydroxyurea in EYA4-depleted cells, enhanced levels of unresolved DNA breaks are observed. Without wishing to be bound by theory, EYA4 plays a crucial role in the repair of replication-associated DNA damage.

Taken together, these data indicate that EYA4 is a novel oncogene in breast cancer and could play a role in cell cycle maintenance. Without wishing to be bound by theory, this makes EYA4 an attractive, druggable target in cancer treatments, especially in triple-negative breast cancer, to limit metastasis and overcome chemotherapy resistance.

### a. MATERIALS AND METHODS

### i. Plasmids

MISSION TRC2 pLKO.5-Puro empty vector (EV) or EYA4 shRNA constructs (shRNAT, TRCN0000244430; shRNA2, TRCN0000218273; shRNA3, TRCN0000244429) were obtained from Sigma-Aldrich. pcDNA3.1-nV5 EYA4 full length (FL) and pDEST26-His EYA4 FL were cloned and sent for sequencing. pcDNA3.1-Myc-His EYA4 mutant (3YF281 and pY dead) were obtained from General Biosystems.

### ii. Cell Culture and Maintenance, Transfections, and Stable Cell Line Establishment

HeLa, MDA-MB-231 and MCF-7 cells were sourced from ATCC. Cells were cultured in Dulbecco's Modified Eagle's Medium (DMEM; Gibco) supplemented with 10% fetal bovine serum (FBS) at 37° C. in 5% CO₂ incubators and passaged at 80% confluence or less. MCF-7 cells were supplemented with 10 μg/mL insulin and 1 mM sodium pyruvate. 1.2×10⁶ HEK 293T cells were reverse-transfected using Lipofectamine 2000 reagent (Invitrogen) with pLKO.5 empty vector or EYA4 shRNA constructs and Lenti-vpak plasmids from OnGene to create lentivirus particles. Viruses were harvested at 48 and 72 h post-transfection, filtered through a 0.45 m filter, and used to infect HeLa or MDA-MB-231 cells with 4 μg/mL polybrene. Stable cell lines were selected using 1-2 μg/mL of puromycin. For complementation, stable HeLa or MDA-MB-231 cells expressing shRNAT were transfected with pcDNA3.1 Myc/His containing a mutant version of EYA4 in the S/T domain (Y281F, Y284F, Y285F; referred to as 3YF281) or pY dead (D375N, D377N, T548A, E606Q, E607Q, E608Q)) and selected with 500 μg/mL geneticin. MCF-7 cells were transfected with pcDNA3.1-nV5 EYA4 FL or pDEST26-His EYA4 FL and selected with 500 μg/mL geneticin. MDA-MB-231/Luc and MCF-7/Luc cells stably expressing firefly luciferase were established as described above. HeLa cells were transduced with FUCCI (red/green) plasmids (Sakaue-Sawano A, et al. (2008) Cell 132(3): 487-98) and FACS sorted to select homogenous positive cell populations. The origin of all cells was confirmed by short-tandem repeat (STR) profiling and tested regularly for Mycoplasma contamination.

### iii. RNA Extraction and Quantitative Reverse Transcription PCR (qRT-PCR)

Total RNA was isolated from transfected or transduced cells by phenol-chloroform extraction (TRIzol; Invitrogen) followed by nucleic acid precipitation. The GoScript Reverse Transcription System (Promega) was used to generate first-strand cDNA. Quantitative PCR was performed using TaqMan probes spanning across exons for human EYA4 (Invitrogen Hs01012406_mH) and human 18S (Invitrogen Hs99999901_s1) to amplify 70 bp and 187 bp fragments, respectively. The relative expression of EYA4 was determined using the 2^{-ΔΔCt} method with 18S as an endogenous control for normalization.

### iv. Immunoblotting

Immunoblotting analysis was conducted according to our standard procedures (C. Wiese et al., (2007) Mol Cell 28: 482-490). Cells were collected and lysed in RIPA buffer (10 mM Tris-HCl (pH 8.0), 1 mM EDTA, 0.5 mM EGTA, 1% Triton X-100, 0.1% sodium deoxycholate, 0.1% SDS, 140 mM NaCl) supplemented with cOmplete Mini EDTA-free protease inhibitor cocktail (Roche), 1 mM PMSF, 1 mM Na₃VO₄, 1 mM NaF, 1 mM benzamidine and 0.025 U/μL benzonase, followed by sonication for 2 min (40%) in an ultrasonic water bath (Sonics Vibra-Cell VCX400). Proteins were resolved in 4-20% Mini-Protean TGX gels (Bio-Rad) and transferred to Immobilon-P PVDF membranes (Merck). Membranes were then blocked with either 5% skim milk or bovine serum albumin (BSA) in TBS-T. Blots were incubated with primary antibody at either 4° C. overnight or room temperature (RT) for 2 h, washed, then incubated with secondary HRP-conjugated antibodies for 1 h at RT. Bands were visualized using the Clarity Western ECL substrate (Bio-Rad). Primary antibodies: EYA4 (Abcam ab93865), cyclin E1 (HE12; Cell Signaling #4129), CDK2 (78B2; Cell Signaling #2546), p21^{WAF1/CIP1} (12D1; Cell Signaling #2947), p27^{KIP1} (D69C12; Cell Signaling #3686), cyclin A (B-8; Santa Cruz sc-271682), pChk1 (S345) (133D3; Cell Signaling #2348), pChk2 (T68) (Cell Signaling #2661), γH2AX (S139) (Millipore 05-636), PCNA (PC10; Santa Cruz sc-56), GAPDH (14C10; Cell Signaling #2118) and β-Actin (C4; Santa Cruz sc-47778).

### v. Subcutaneous Tumor Xenografts in Immunodeficient Mice

For subcutaneous injections, MCF-7/Luciferase wild type (WT), pcDNA3.1-nV5 EYA4 FL and pDEST26-His EYA4 FL cells (1.0×10⁶) were resuspended in 100 μL of 0.9% (w/v) NaCl and injected in the left mammary fat pad (MFP) of 24 non-obese diabetic/severe combined immunodeficiency gamma (NSG, NOD scid gamma) female mice (6 weeks of age; 8 mice per cell line). A 17β-estradiol pellet (1.7 mg/pellet, 60-days release, Innovative Research of America) was implanted close to the neck using a precision trochar, 24 h prior to MFP injections. Weekly, all mice were weighed, tumor growth was measured by using a caliper and detected in vivo by bioluminescent imaging. For in vivo imaging, mice were first injected with D-luciferin (150 mg/kg, 10 min prior to imaging), anesthetized with 3% isoflurane and then imaged in an IVIS spectrum imaging system (Caliper, Newton, USA). Images were analyzed with Living Image software (Caliper, Newton, USA). Bioluminescent flux (photons/see/sr/cm²) was determined for the tumors. Tumor volume was calculated according to the following formula: (length×width²)/2. MCF-7/Luciferase mice were sacrificed before tumors reached 10 mm (8 weeks post-injection). Harvested tumor tissues were placed in liquid nitrogen and then frozen at −80° C. or fixed in 10% buffered formalin, embedded in paraffin, sectioned, and stained. Antibodies used: anti-Estrogen Receptor (SP1; Roche 790-4324; CC1 64 min), anti-Ki-67 (30-9; Roche 790-4286; CC1 64 min), anti-γH2AX (pS319; Abcam ab2893; CCT 64 min; 1:600).

### vi. Mouse Tail-Vein Assay

MDA-MB-231/Luciferase WT, EYA4 shRNAT and EYA4 shRNA2 cells (1.0×10⁶ cells/100 μL 0.9% (w/v) NaCl) were injected in the lateral tail-vein of 9 female NOD scid gamma mice (6 weeks of age; 3 mice per cell line). For complementation, MDA-MB-231/Luciferase WT, EYA4 shRNA1 and pcDNA3.1-Myc-His EYA4 mutant (3YF281 and pY dead) cells (1.0×10⁶ cells/T100 μL 0.9% (w/v) NaCl) were injected in the lateral tail-vein of 22 female NSG mice (7 weeks of age; 7 WT mice and 5 mice per cell line). Mice were detected every week for metastatic foci by bioluminescent imaging as described above. MDA-MB-231/Luciferase mice were monitored and culled 4-5 weeks post-injection. Bioluminescent flux (photons/s/cm²/sr) was determined. Organs in which metastatic foci were observed were harvested and fixed in 4% PFA, followed by 70% EtOH, then embedded in paraffin, sectioned, and stained. Antibodies used as described above.

### vii. Cell proliferation assay

Cells were seeded in a 96-well plate at 2.0×10³ cells/well. Phase contrast images of cells were acquired every 2 h using an IncuCyte Zoom (Essen BioScience) live imaging system. Proliferation was measured as a percentage of confluency.

### viii. In Vitro Migration Assay

Cells were cultured in a 96-well plate for 24 h to achieve 100% confluency. An IncuCyte Woundmaker was used to make a scratch in the cell monolayer. Cells were then incubated in serum-free media and automatically imaged every 2 h using an IncuCyte Zoom (Essen BioScience) live imaging system. The scratch gap width and confluence were measured at each time point and compared between groups.

### ix. Apoptosis

HeLa cells were seeded in a 96-well plate (100 cells/well). After 24 h, annexin V (red) reagent was added according to manufacturer's protocol (IncuCyte). Images (phase contrast/orange) were acquired every 2 h using an IncuCyte SX5 (Sartorius) live-cell imaging system. Apoptosis was measured as total integrated intensity (OCU×m²/image).

### x. Double Thymidine Block and Cell Cycle Progression (Flow Cytometry)

HeLa cells were synchronized in early S-phase by a double thymidine block. Briefly, cells were blocked with 2 mM thymidine for 18 h, released for 9 h, and blocked again with 2 mM thymidine for 17 h. After the second block, cells (asynchronized and synchronized) were released and collected according to time points, then fixed in ice-cold 70% ethanol at −20° C. for at least 24 h. DNA was stained with 38 mM trisodium citrate, 100 μg/mL RNase A and 150 μg/mL propidium iodide (PI) for 1 h at RT. A DNA control PI (trout erythrocytes) was used as an internal control to normalize the cell cycle. Data were collected using a CytoFLEX Flow Cytometer (Beckman Coulter) and cell cycle profiles were analyzed with FlowJo to determine the percentage of cells in GI, S and G2/M. 10,000 events were collected, and aggregated cells were gated out.

### xi. FUCCI

HeLa FUCCI cells stably transfected with empty vector or EYA4 shRNAs were seeded in a 96-well plate (100 cells/well). Phase contrast and green/orange images were acquired every 2 h to monitor cell cycle progression using an IncuCyte SX5 (Sartorius) live-cell imaging system. Images were analyzed using cell-by-cell analysis software and population subsets were classified based on green and red fluorescence. G1 phase (red), G1-S transition (green+red), S/G2/M phase (green) and M-G1 transition (non-fluorescent) (19).

### xii. Indirect Immunofluorescence

Indirect immunofluorescence was performed as described elsewhere (B. de la Pe5a Avalos, E. Dray. Journal of Visualized Experiments 160, e61447 (2020)). Cells were grown on coverslips for 24 h and treated with 4 Gy γ-irradiation (Gammacell40 Exactor unit) or 4 mM hydroxyurea. Cell nuclei were pre-extracted with nuclear extraction buffer (NEB; 10 mM PIPES (pH 6.8), 100 mM NaCl, 300 mM sucrose, 3 mM MgCl₂, 1 mM EGTA (pH 8.0), 0.5% Triton X-100) for 2 min at RT then fixed with 4% paraformaldehyde (PFA) for 10 mn at 4° C. Nuclei were blocked in 5% BSA and 0.3% Triton X-100 in PBS, immunoblotted with a primary antibody (1:500 in dilution buffer; 1% BSA and 0.3% Triton X-100 in PBS), followed by secondary antibody (2 μg/mL in dilution buffer). DNA was counterstained with DAPI. Slides were viewed on an Olympus FV3000 confocal microscope. Primary antibodies: CENP-F (H-260; Santa Cruz sc-22791), γH2AX (S139) (Millipore 05-636). Secondary antibodies: α-Rabbit (Abcam ab150081, Alexa Fluor 488), α-Mouse (Abcam ab150103, Alexa Fluor 647). Nuclear foci quantification was performed using CellProfiler.

### xiii. MTT Cell Cytotoxicity Assay

For genotoxic stress, cells were seeded into 96-well plates (200 cells/well). Twenty-four hours after seeding, increasing concentrations of ATR inhibitor (AZ20) or hydroxyurea were added to the culture (24 h pulse). Cell cytotoxicity was measured after 96 h following manufacturer's protocol (Abcam ab211091). Briefly, 50 μL serum-free media (no phenol red) and 50 μL MTT reagent was added to each well and incubated at 37° C. for 3 h. MTT media was replaced with 150 μL of MTT solvent and incubated with agitation for 15 mn. Absorbance was measured at 590 nm. The cell viability was calculated using the following equation:

$$Cell viability ( \% ) = \frac{O {D}_{treated}}{O {D}_{control}} \times 100$$

OD_{treated} and OD_{control} represented the absorbance of sampled and control, respectively.

### xiv. EdU Incorporation

HeLa control and EYA4 knockdown cells (4.0×10⁴ cells/well) were seeded in 12-well plates with coverslips for 24 h. 5-ethynyl-2′-deoxyuridine (EdU) incorporation was performed according to manufacturer's protocol (Base Click). Briefly, cells were treated with 4 mM hydroxyurea for 2 hours, released for 10 minutes, then labeled with 10 μM of EdU for 30 min at 37° C., then fixed with 4% PFA for 10 min at 4° C., followed by permeabilization with 0.3% Triton X-100 in PBS for 20 min at RT. Reaction cocktail with 6-FAM azide was added to fixed cells and incubated for 30 min at RT. DNA was counterstained with DAPI. Slides were viewed on an Olympus FV3000 confocal microscope. EdU-stained cells were quantified using CellProfiler.

### xv. DNA Fiber Assay

Exponentially growing HeLa cells (3.0×10⁵) were labeled with a 5-iodo-2′-deoxyuridine (IdU; 50 μM) pulse for 30 min. After labeling, cells were harvested, embedded in agarose and DNA was prepared then combed onto silanized coverslips using the FiberComb Molecular Combing System (Genomic Vision). Following combing, coverslips were baked for 2 h at 65° C. Combed DNA fibers were denatured with 0.5 M NaOH+1 M NaCl for 8 min at RT, neutralized with PBS (3 times, 3 min), then dehydrated in ethanol (70%-90%-100%, 3 min each), and air-dried. Combed DNA was blocked with BlockAid blocking solution (Invitrogen B10710) for 15 min at RT, followed by immunostaining with mouse α-BrdU (to detect IdU; BD Biosciences 347580) for 1 h at 37° C., washed with PBS-T, and probed with secondary antibody (α-mouse Cy3, SIGMA C2181) for 45 min at 37° C. Single-stranded DNA was counterstained with α-ssDNA mouse antibody (DSHB University of Iowa) for 2 h at 37° C., followed by α-mouse BV480 (Jackson ImmunoResearch 115-685-166) for 45 min at 37° C. Coverslips were washed in PBS, subjected to a graded ethanol series, air-dried, and then mounted with 25 μL of Vectashield mounting medium (Vector Laboratories). DNA fiber images were acquired on an Olympus FV3000 confocal microscope. Track lengths were measured with ImageJ. To calculate replication fork speed, the following equation was used to convert fork length from m to kb/min: length μm×2/labeling time in min=fork speed kb/mm (conversion factor of 2 kb/m specific for DNA combing method).

### xvi. Statistical Analysis

The statistical analyses were conducted using GraphPad Prism 9 and a p<0.05 was considered statistically significant. Student's t-test was used to test for significant differences between groups, considering a normal distribution. Unpaired two-tailed tests were applied to all data if not specified. Samples sizes were chosen according to previously published methods where significant biological conclusions were reported.

### b. EYA4 IS A NOVEL BREAST CANCER GENE

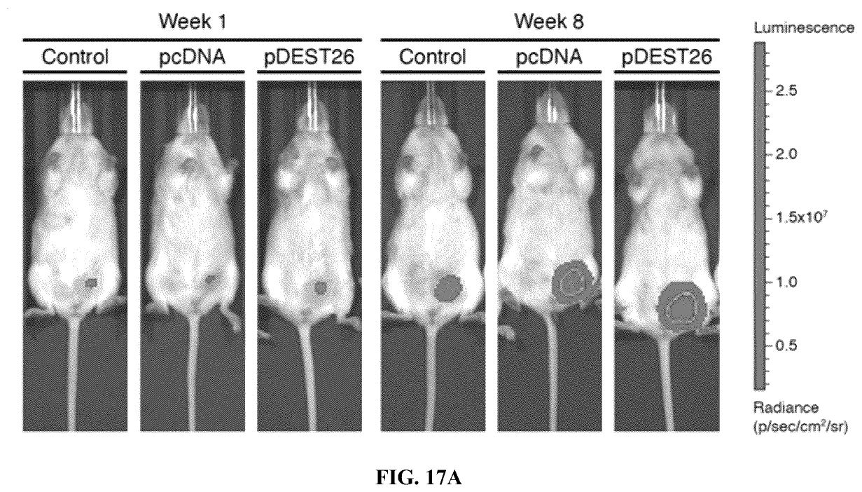

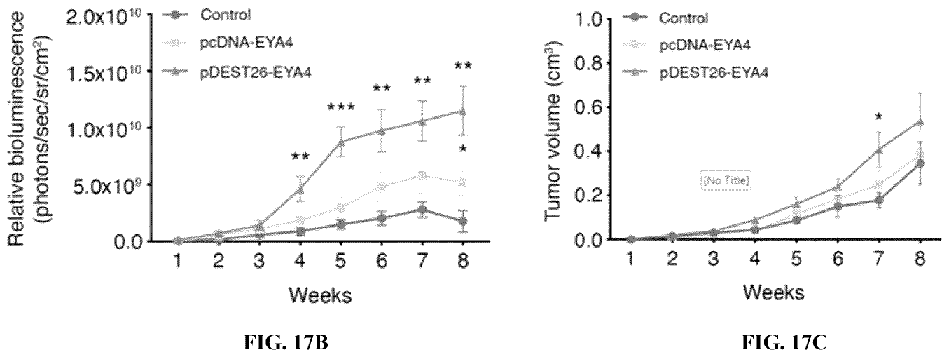

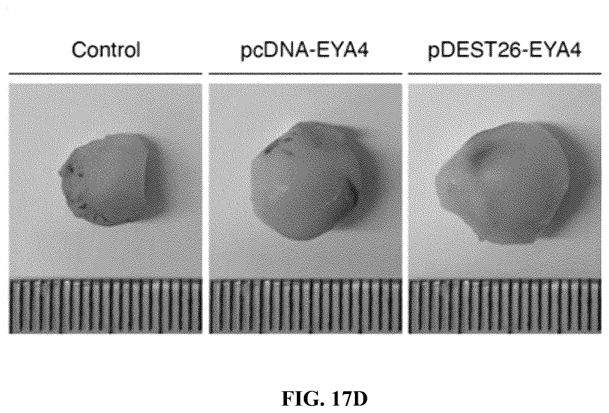

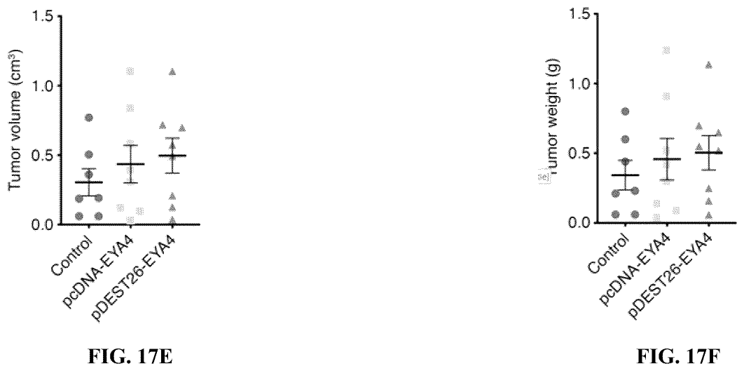

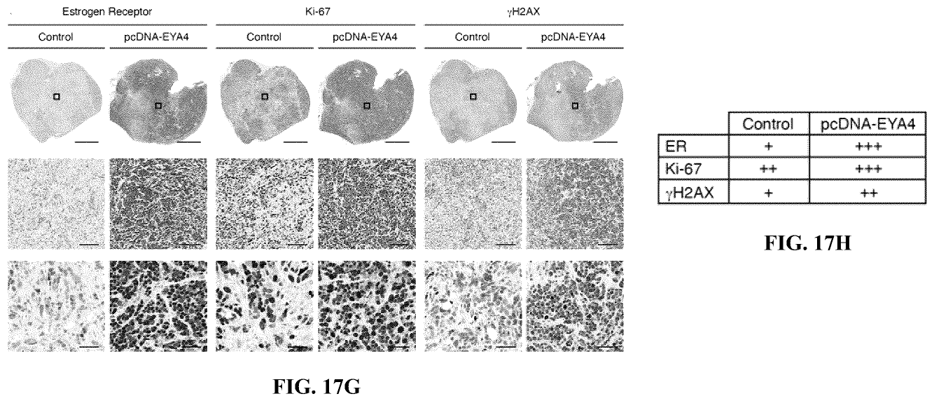

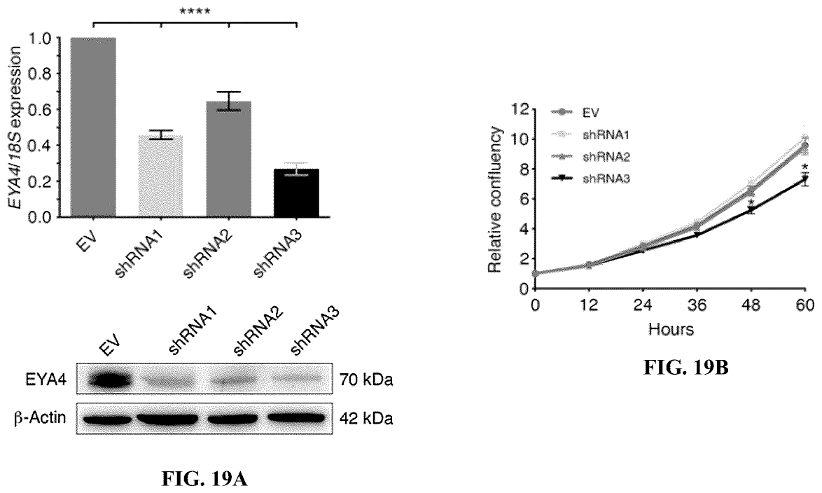

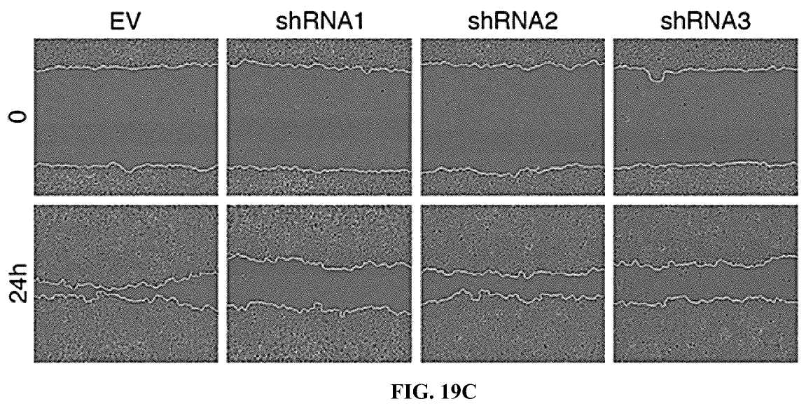

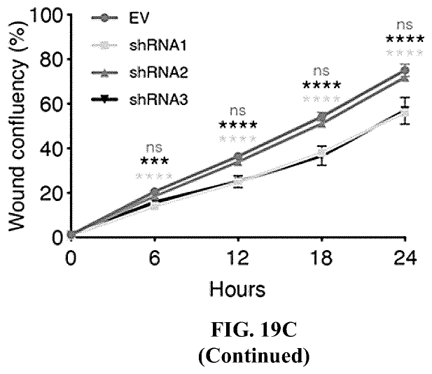

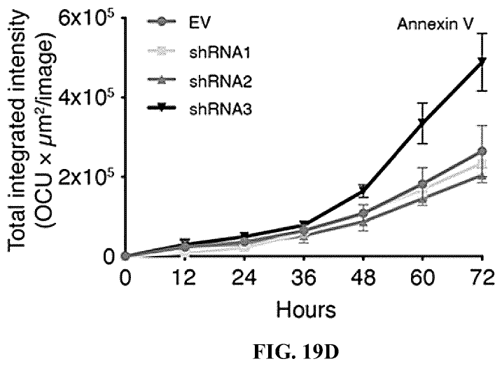

EYA4 was investigated as to whether it is expressed in specific breast cancer subtypes using real-time quantitative PCR and immunoblotting in several breast cancer cell lines (FIG. 23B and FIG. 23C). The expression of EYA4 varied greatly across cell lines, however, the triple-negative breast cancer cell line MDA-MB-231 showed the highest endogenous expression of EYA4. In most mouse strains, knockout of EYA4 is lethal shortly after birth (F. F. Depreux et al., (2008) The Journal of Clinical Investigation 118: 651-658) and is toxic in several lung cancer cell lines (I. M. Wilson et al., (2014) Oncogene 33: 4464-4473) and other cell lines that were tested. Using short-hairpin RNAs (shRNAs), EYA4 expression could be significantly decreased in MDA-MB-231 cells (FIG. 23D) or in HeLa cells (FIG. 19A). The most efficient hairpin, shRNA3, induces cell death in MDA-MB-231, indicating that EYA4 is essential in these cells. In parallel, EYA4 was over-expressed using two different vectors (FIG. 23E) in the ER⁺/PR⁺ breast cancer cell line, MCF-7, which expresses low or no detectable endogenous EYA4 (FIG. 23B and FIG. 23C and Cancer Cell Line Encyclopedia, https://sites.broadinstitute.org/ccle). The effects of EYA4 deregulation on primary cancer growth and metastasis were assessed in vivo using luciferase-expressing cell lines. A human tumor xenograft model was established using NOD scid gamma mice. MCF-7/Luc wild type (WT) and EYA4 over-expressing cells were injected subcutaneously into the left mammary fat pad (MFP) of female mice supplemented with 17β-estradiol and monitored by caliper measurement and in vivo imaging for 8 weeks. Following an intraperitoneal injection with D-luciferin (150 mg/kg), the firefly luciferase enzyme catalyzes this substrate, which results in light photons that are captured by a charge-coupled device (CCD) camera mounted within an IVIS® Spectrum Imaging System (Carceles-Cordon M, et al. Bio-Protocol. 2016; 6(6)). As shown in FIG. 17A and FIG. 17B, the bioluminescence intensity (BLI) signal measurement confirmed tumor engraftment for all mice. Primary tumors show a significant increase in volume when EYA4 is over-expressed. BLI signal correlated with caliper measurements as observed in FIG. 17C, and with tumor volume and weight (FIG. 17E and FIG. 17F) once surgically removed postmortem (FIG. 17D). EYA4 over-expression leads to a more aggressive breast cancer, as observed by immunohistochemistry (IHC) staining (FIG. 17G). These results correspond with previous reports that in MPNST, EYA4 is dramatically upregulated in cells and primary tumors, and its depletion leads to reduced cell adhesion and migration in vitro and has an inhibitory effect in tumorigenesis in vivo (Miller S J, et al. Oncogene. 2010; 29(3):368-79).

Estrogen receptor alpha (ER-α) co-stain was used to validate human cells. Interestingly, cells expressing high levels of EYA4 also showed high expression of ER-α, the proliferation-related antigen Ki-67, and γH2AX, a marker of DNA damage (FIG. 17G and FIG. 17H). ER-α has a well-established role in supporting estrogen-dependent breast tumor growth through its association with aberrant proliferation (up-regulating Ki-67), which can result in the accumulation of random DNA mutations (marked by γH2AX), and when highly expressed it is associated with poor prognosis in breast cancer (Thomas C, et al. Nature Reviews. 2011; 11:597-608, Liao X—H, et al. FEBS Journal. 2014; 281:927-42), which can explain the aggressive breast cancer subtype observed when EYA4 is over-expressed.

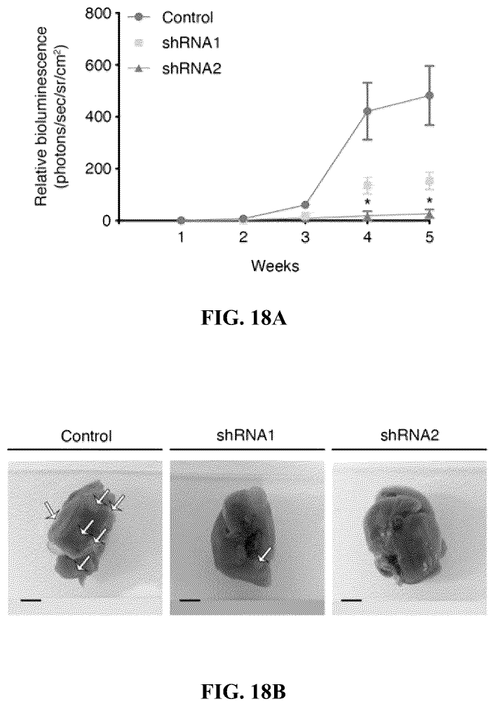

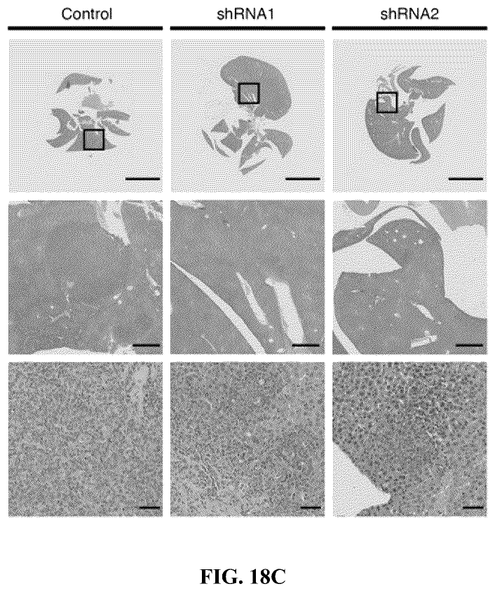

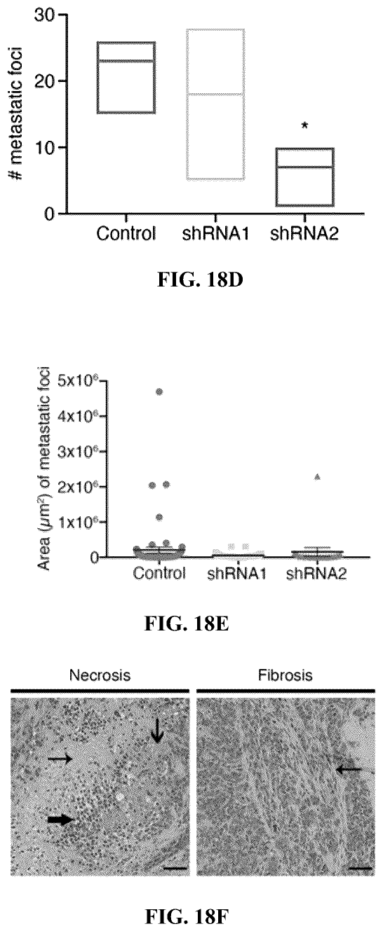

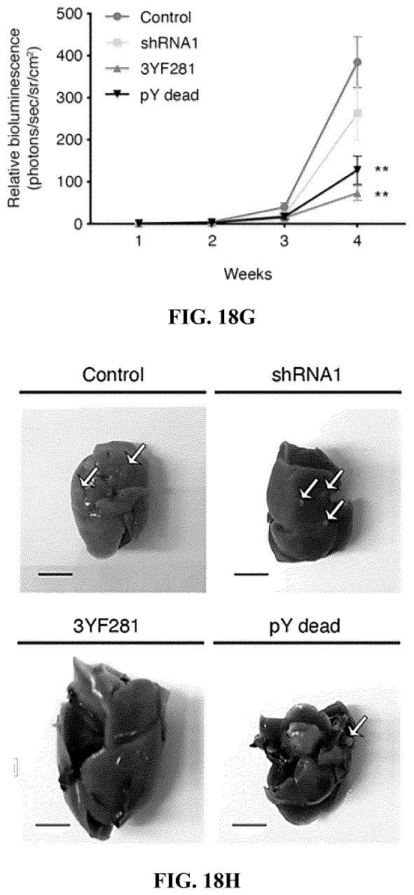

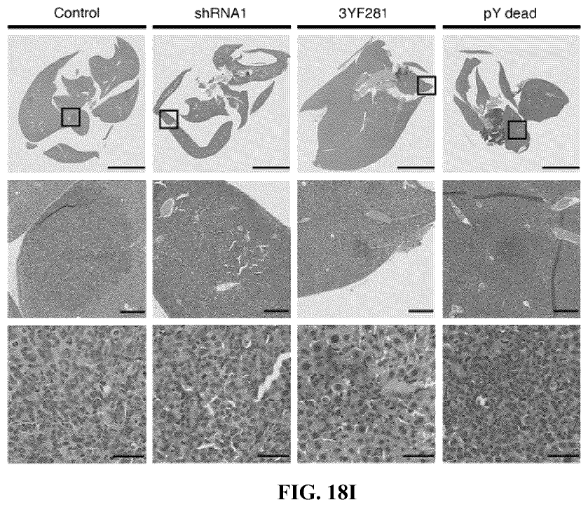

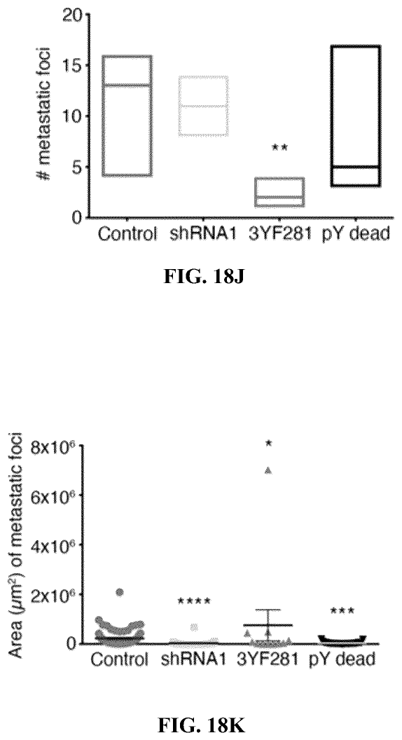

Since breast cancer subtypes are associated with unique patterns of metastatic spread, the metastatic capacity was assessed utilizing MDA-MB-231 stably expressing firefly luciferase. MDA-MB-231/Luciferase WT cells and cells in which EYA4 was stably knocked down (shRNA1 and shRNA2) were injected into the tail vein and monitored by in vivo imaging over 5 weeks. While WT and EYA4-depleted cells colonized the lungs as expected following systemic injection, a decrease in BLI signal was observed in mice injected with EYA4-depleted cells compared to the control (FIG. 18A). This was directly linked to a lesser number and a decrease in the area of metastatic foci observed in livers as revealed by histological analyses (FIG. 18B-E). Importantly, these IHC analyses also showed significant areas of central necrosis with inflammatory cells and blood vessel congestion (left panel) and scant fibrosis (right panel) was observed in the control group but not in the EYA4 knockdown mice (FIG. 18F). This particular observation could be due to the role that EYA4 plays in innate immune system regulation by enhancing the expression of IFN-β and CXCL10, in response to DNA stimulation (Y. Okabe, et al., Nature 460, 520-524 (2009)). In cancer cells, the cGAS-STING pathway is constitutively activated, inducing chronic IFN-β expression, triggered by the accumulation of DNA damage due to replication fork collapse or reactive oxygen species (ROS) that leads to the presence of DNA in the cytoplasm (Cheon H, et al. Trends in Cancer. 2023; 9(1):83-92). Altogether, the data suggest that EYA4 is a driver of breast cancer and that decreasing its expression reduces tumor and metastatic burdens.

### c. THE S/T PHOSPHATASE DOMAIN OF EYA4 CONTRIBUTES TO BREAST Cancer Development

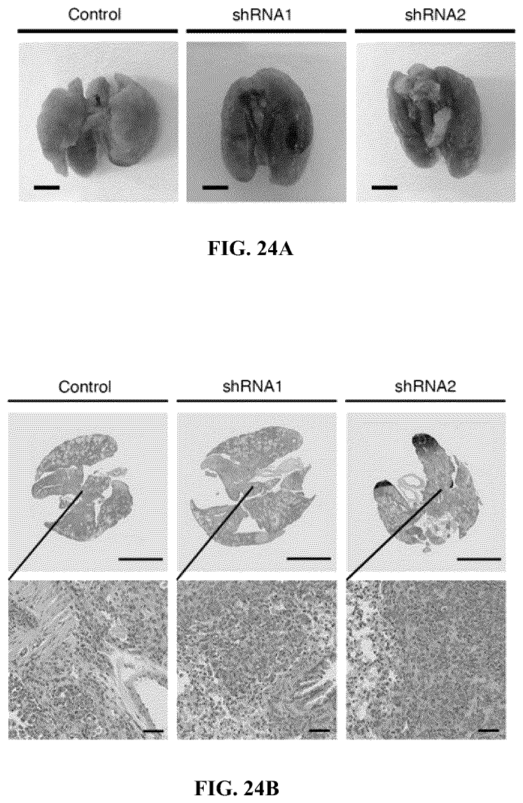

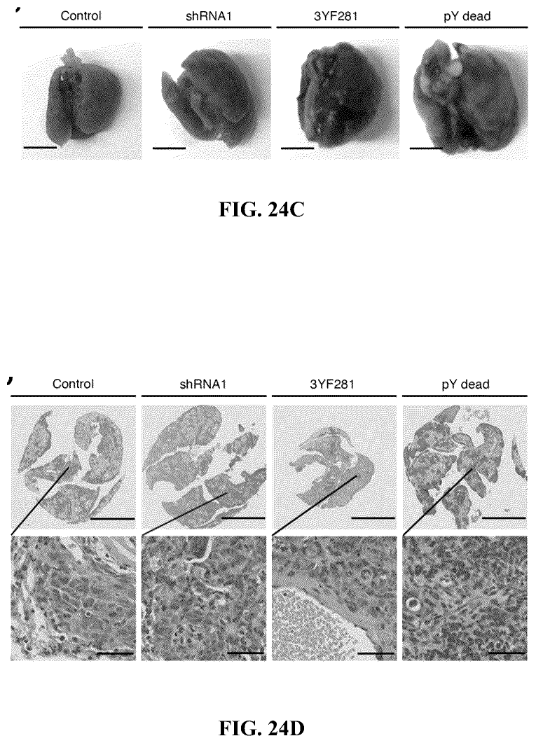

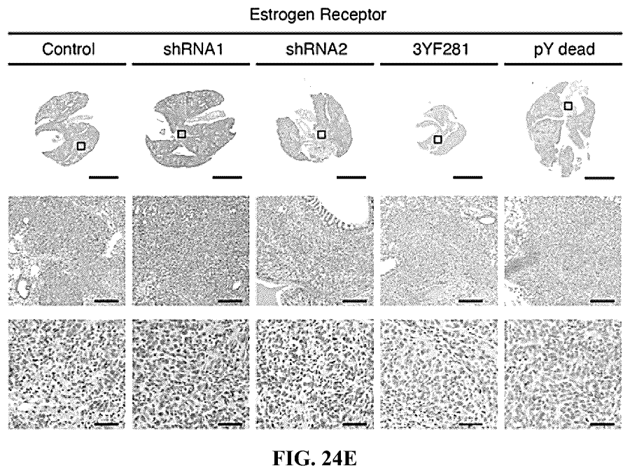

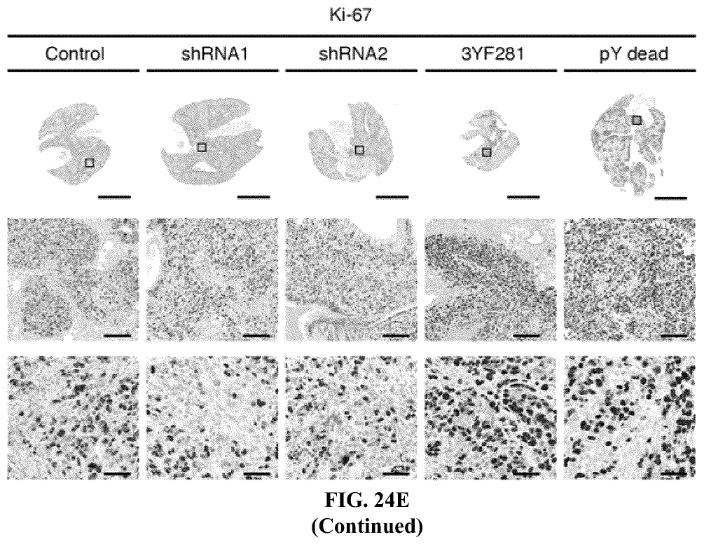

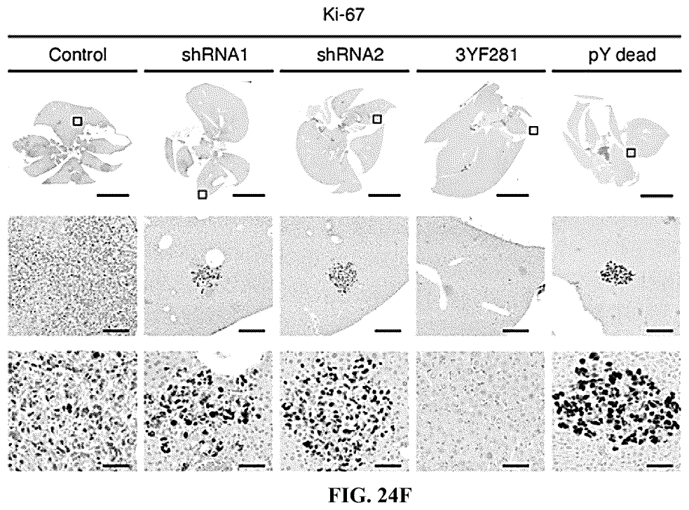

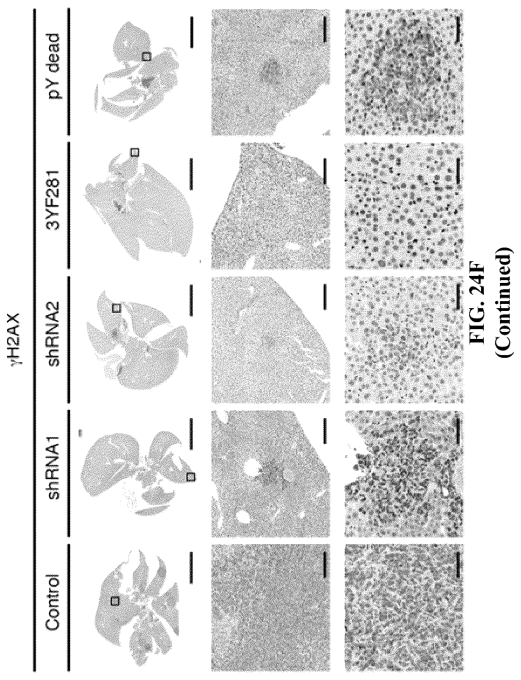

EYA4 possesses both serine/threonine (S/T) and tyrosine (Y) phosphatase activities (FIG. 23A) (Rebay I. Molecular and Cellular Biology. 2016; 36(5):668-77). To investigate the relevance of these activities on tumor growth, MDA-MB-231/Luc cells expressing either EYA4 mutated in the S/T domain (Y281F, Y284F, Y285F; henceforth, 3YF281) or the pY dead combination mutant (D375N, D377N, T548A, E606Q, E607Q, E608Q) were injected into the tail-vein and monitored by in vivo imaging for 4 weeks using the luciferase reporter. The phosphatase mutants (3YF281 and pY dead) caused even more significant outcomes that EYA4 depletion, especially the 3YF281 mutant. Both EYA4 phosphatase mutants did not complement EYA4 depletion with shRNA1, as observed by both BLI signal (FIG. 18G) and by metastatic foci observed in livers (FIG. 18H-K). However, the serine/threonine phosphatase activity of EYA4 (3YF281) is the one that shows more significant outcomes, as observed not only by decreased tumor burden to lungs (FIG. 24C-E), but also by a lesser number of metastatic foci to the liver, with an average of 2 foci for 3YF281, compared to 6 for EYA4 shRNA2 and 7 for pY dead (FIG. 18J). In addition, as observed by IHC staining, when a metastatic site is observed (marked by H&E) in mouse injected with 3YF281 cells, there is no stain by Ki-67 or γH2AX (FIG. 24F). For γH2AX, only a background level (mouse cells stained), can be observed. Notably, all mice injected with 3YF281 cells showed liver enlargement and hyperplasia (FIG. 18H and FIG. 18I), which could be driven by an increased hepatocyte number, prompting further investigation. Without wishing to be bound by theory, these data suggest that the serine/threonine phosphatase activity of EYA4 is essential for breast cancer progression and metastasis.

### d. EYA4 PROMOTES CELL PROLIFERATION AND MIGRATION

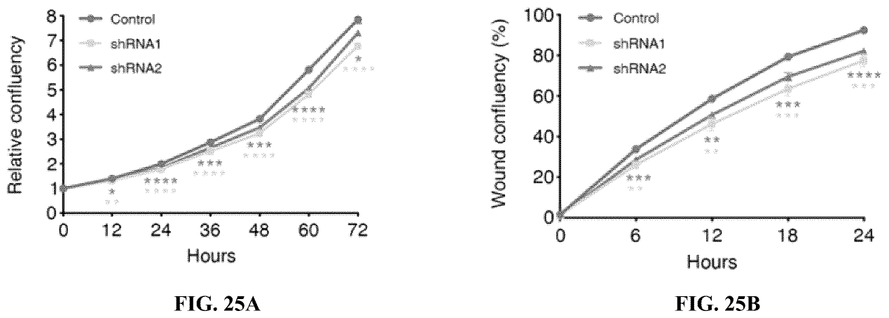

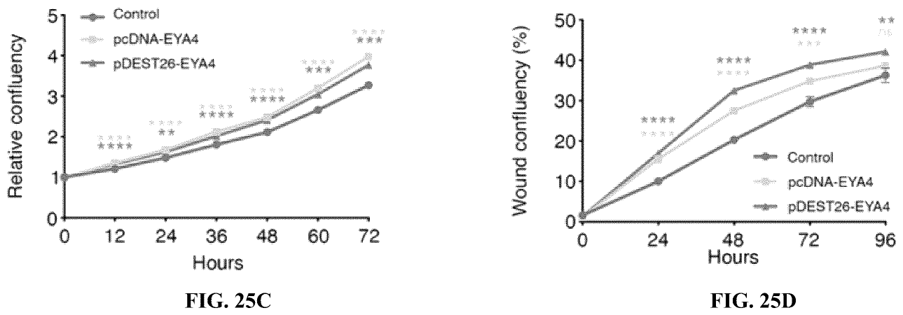

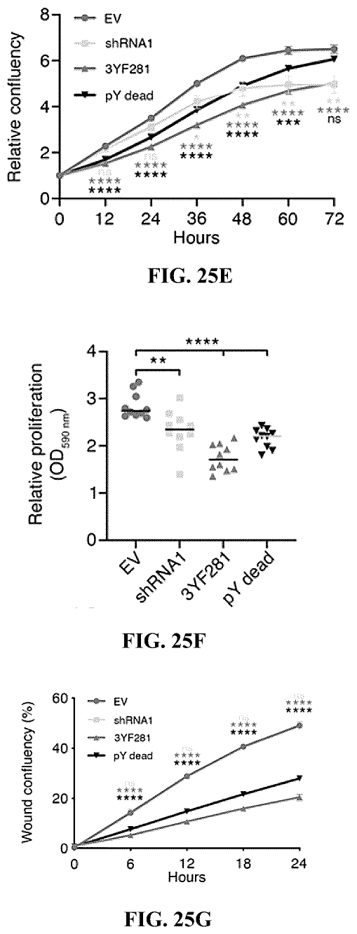

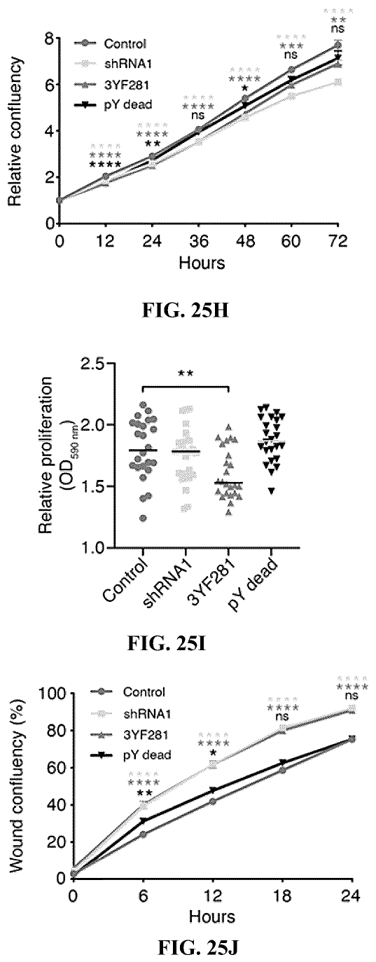

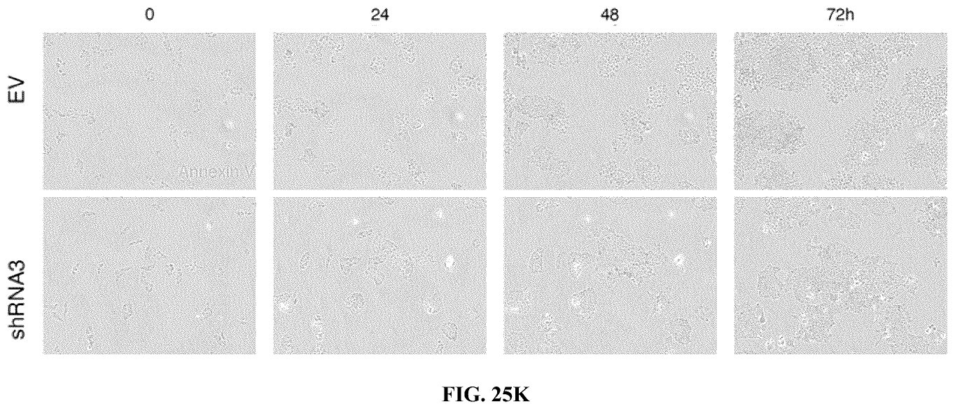

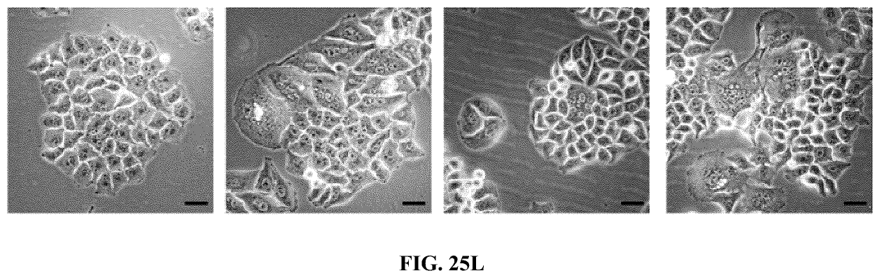

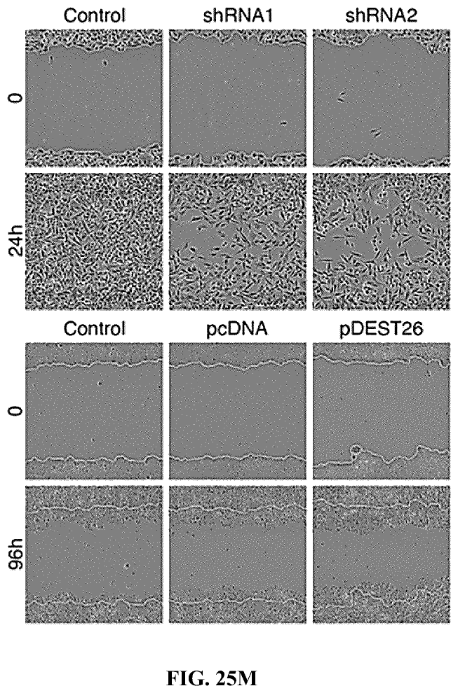

Without wishing to be bound by theory, one simple explanation for variations in primary tumor sizes is the accumulation of larger cells (Qiu J, et al. (2022) Cancer Management and Research 14: 2235-41; Zhou X, et al. (2022) Frontiers in Cell and Developmental Biology 10) or increased proliferation rates. Uncontrolled and unlimited cell proliferation is a hallmark of cancer (Hanahan D. (2022) Cancer Discovery 12(1): 31-46) and another member of the Eyes Absent family, EYA2, has been shown to increase cell proliferation in lung cancer (Li Z, et al. (2017) Oncotarget 8(67): 110837-48). Stable knockdowns were generated in HeLa cells, using three independent short-hairpin RNAs, and a significant decrease in EYA4 protein levels was achieved (FIG. 19A). Growth rates were followed by live-cell imaging. In both, HeLa (FIG. 19B) and MDA-MB-231 (FIG. 25A) cells, depletion of EYA4 led to lower proliferation rates compared to control. On the contrary, the over-expression of EYA4 in MCF-7 leads to higher proliferation rates when compared to control (FIG. 25C), suggesting that EYA4 promotes cell proliferation. In addition, the effect of EYA4 on cell migration was investigated by comparing the number of control, EYA4 knocked down and EYA4 over-expressing cells at the scratch wound at different time points by live-cell imaging. HeLa (FIG. 19C) and MDA-MB-231 (FIG. 25B) cells depleted for EYA4, exhibited significantly lower migratory capacity relative to cells expressing the empty vector (EV) control, whilst EYA4 over-expression in MCF-7 (FIG. 25D) primes the migration capacity of cells, indicating that EYA4 plays a role in driving cell migration. EYA4 phosphatase mutants, specially 3YF281, display a phenotype comparable, or even more dramatic, than EYA4 depleted cells when tested for proliferation and migration capacities in HeLa cells (FIG. 25E-G), showing a significant decrease for both. However, the same phenotype was not observed in MDA-MD-231 cells (FIG. 19H and FIG. 19J), suggesting that the role in cell migration might be cell line dependent. As the apparent slower proliferation being caused by cell death cannot exclude, HeLa control and EYA4 knockdown cells were followed after the addition of the apoptosis marker, annexin V. Compared to HeLa control cells, EYA4 shRNA3 showed a slight increase in apoptosis in normal growth conditions (FIG. 19D and FIG. 25K), which could explain, at least partially, the slower proliferation rate observed for shRNA3. The increase in apoptosis observed in HeLa EYA4 shRNA3 cells reflects the fact that this hairpin could not be used in MDA-MB-231 cells, as severe knockdown of EYA4 is incompatible with cell viability.

### e. EYA4 PERTURBS CELL CYCLE PROGRESSION

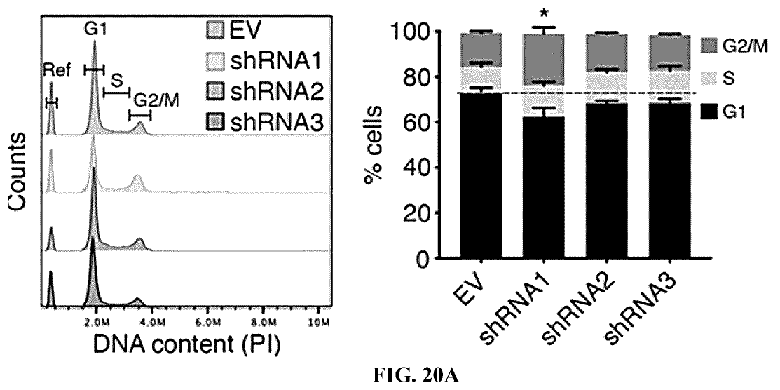

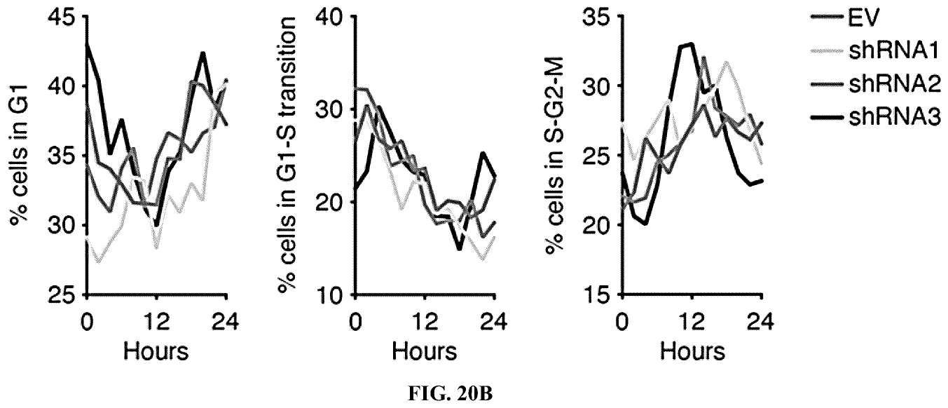

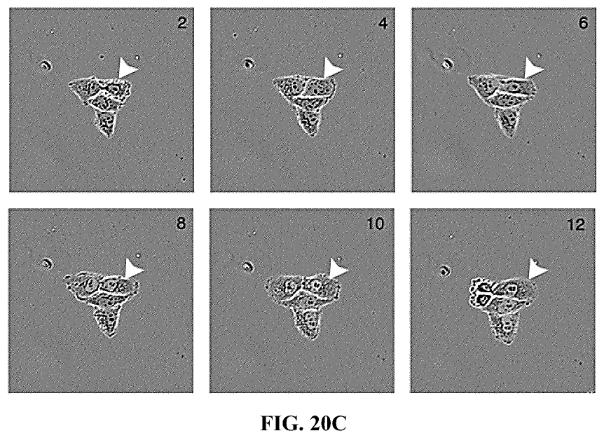

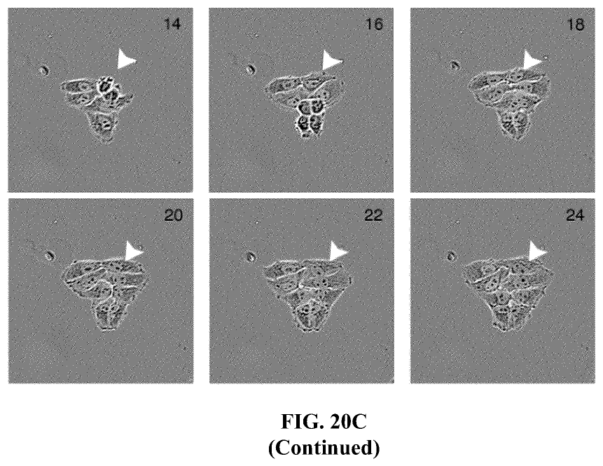

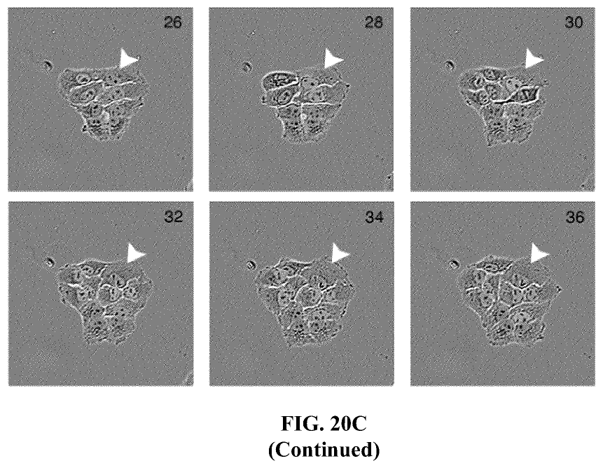

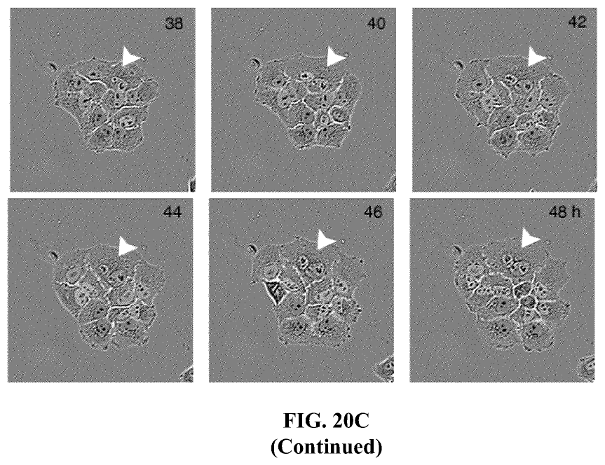

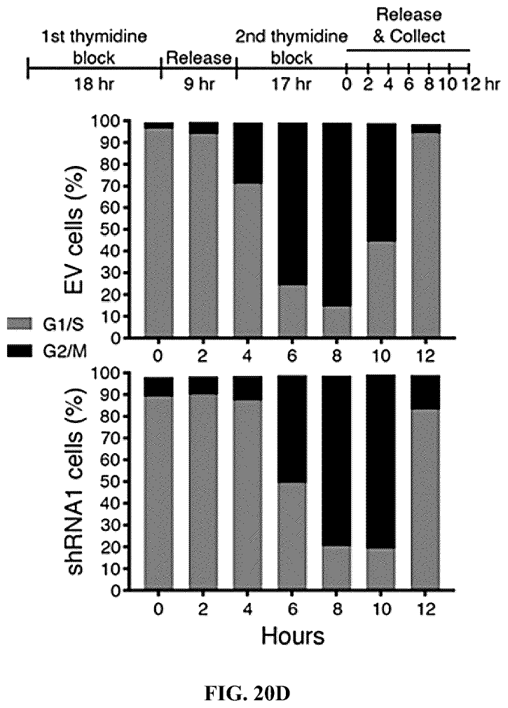

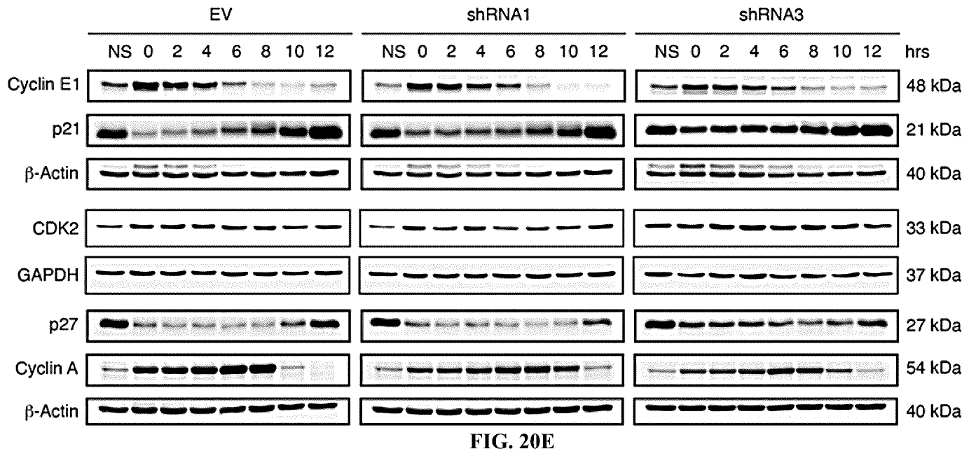

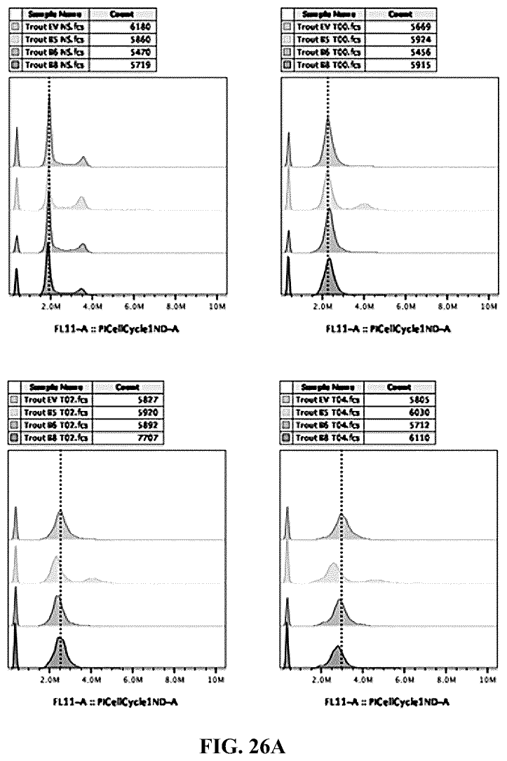

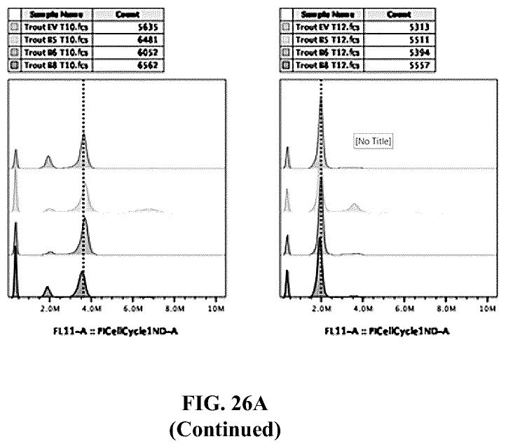

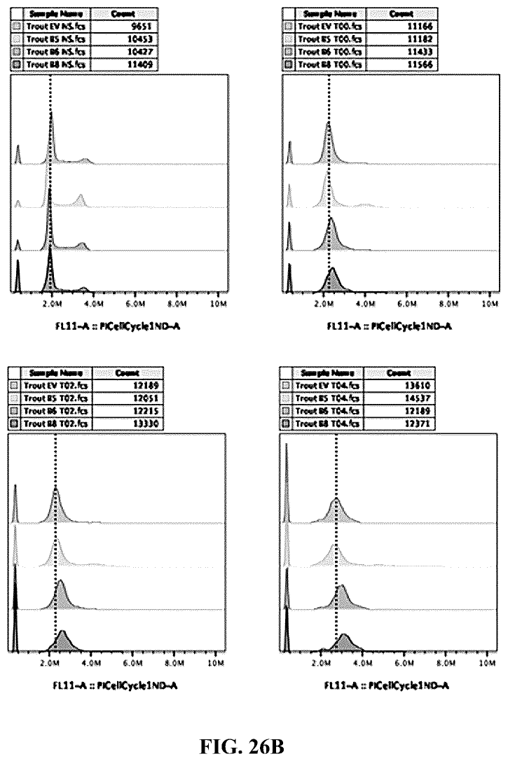

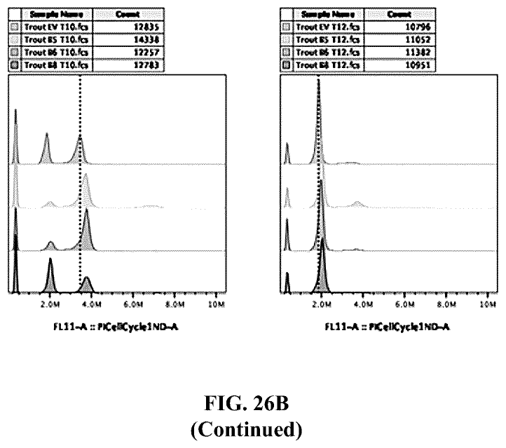

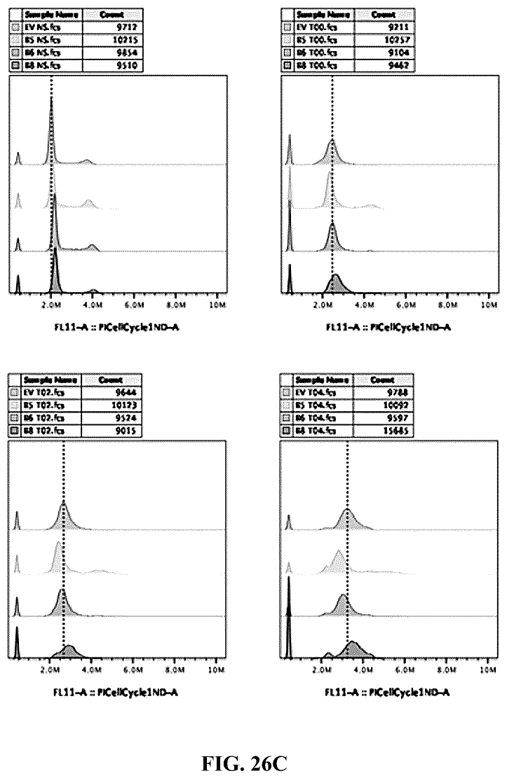

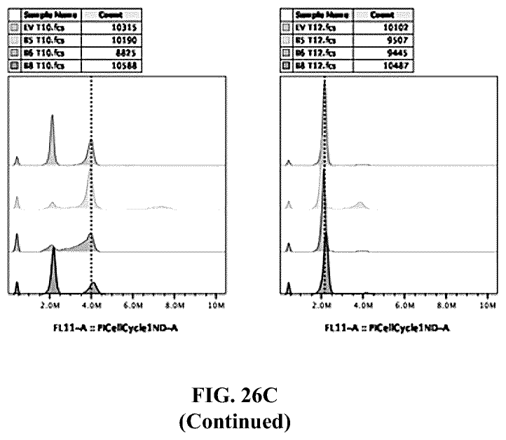

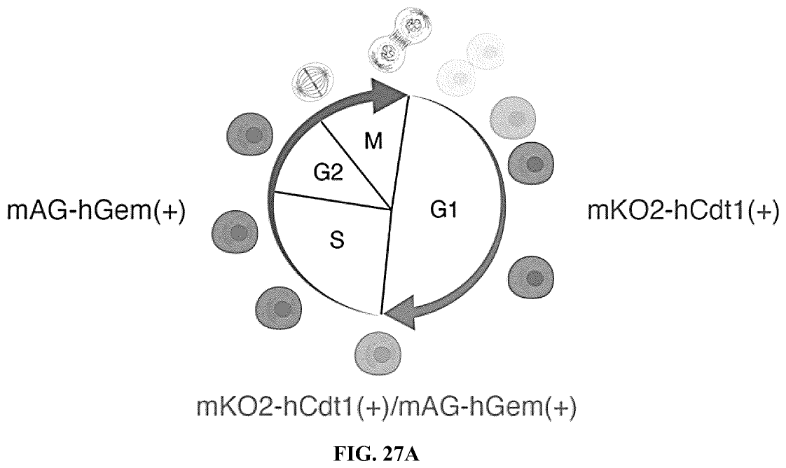

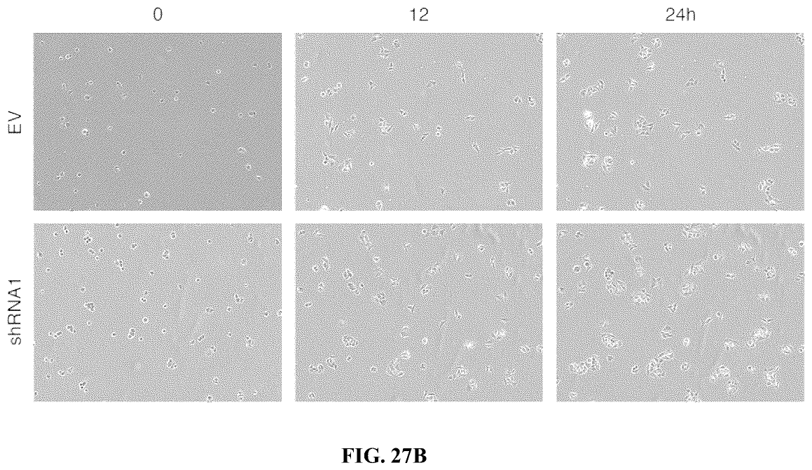

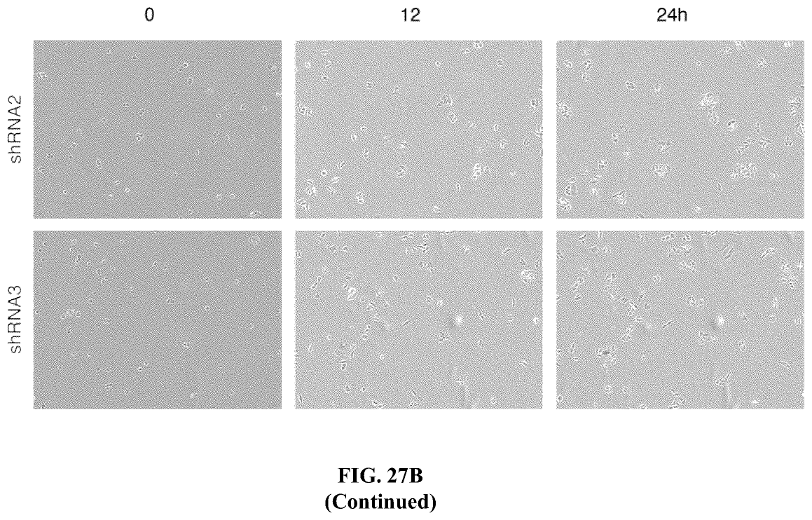

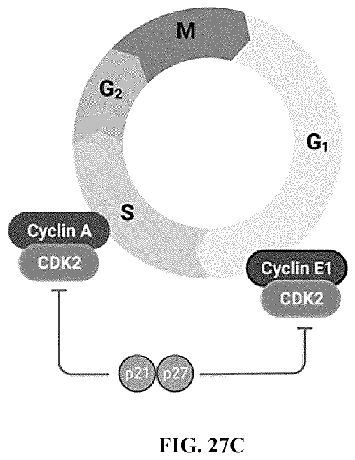

Cell cycle is tightly regulated via checkpoints that are activated by DNA damage, low nutrient content, or other endogenous and external stresses. Aberrant cell cycle progression tends to result in genome instability and contributes to cancer progression. To determine how EYA4 might affect cell cycle progression, flow cytometry was used to profile asynchronous populations of either control or EYA4-depleted cells (FIG. 20A). A slight increase (2-3%) in S-phase was observed when EYA4 is silenced and a significant increase (8%) in the G2/M population for shRNA1 (FIG. 20A), when compared to empty vector control, which, without wishing to be bound by theory, suggested a delay in cell cycle progression upon EYA4 depletion. However, shRNA3 does not show a significant increase in G2/M, which could be explained due to its characteristic phenotype (enlarged, flat and multinucleated cells, FIG. 25L), and this subpopulation could have been gated out by flow cytometry (raw data in FIG. 26A-C). The FUCCI system (Sakaue-Sawano A, et al. (2008) Cell 132(3): 487-98) was used and live-cell imaging (FIG. 20B and FIG. 27A) to overcome these technical issues and profile single cells. A subtly different behavior was observed for EYA4 shRNA3, especially when it comes to cells in S-G2-M (FIG. 20B). This correlates with cells depleted for EYA4 (especially with shRNA3) undergoing endoreplication (FIG. 20C). Endoreplication refers to a cell cycle variant that only consists of the G and S phases, during which cells replicate their DNA content without dividing, thus giving rise to polyploid cells (Shu Z, et al. (2018) Trends in Cell Biology 28(6): 465-74; Lee H O, et al. (2009) Genes & Development 23: 2461-77). The result is either a cell that maintains separate nuclei and remains multinucleated, due to a process called endomitosis, or a cell with an enlarged-single nucleus containing all the DNA, derived from a process called endocycling (Shu Z, et al. (2018) Trends in Cell Biology 28(6): 465-74). As described above, shRNA3 cells tend to be enlarged and multinucleated, which is characteristic of endomitosis, a major form of endoreplication in which mitosis is initiated but not completed (green/non-fluorescent/green; white arrowhead; FIG. 20C). The endoreplication and consequent polyploidy observed, which can occur in response to stress, is a phenomenon that has been linked to cancer progression and chemotherapy resistance (Tagal V, et al. (2021) Cancer Research 81(2): 400-13).

### f. EYA4 INDUCES CELL CYCLE ARREST

The most common change leading from a mitotic to an endoreplication cycle is a switch in activation/inactivation of cyclins and cyclin-dependent kinases (CDKs), key regulators of cell cycle progression (Gandarillas A, et al. (2018) Cell Death & Differentiation 25: 471-6). To investigate if EYA4 expression impacts individual phases of the cell cycle, cells were arrested in early S-phase with a double thymidine block (FIG. 20D) and assessed for cell cycle progression. Propidium iodide (PI) staining of the DNA and flow cytometry in HeLa cells showed that EYA4 decrease (shRNA1) leads to a delay in S-phase restart compared to control (FIG. 20D). Upon release, 74.9% of control cells entered G2/M after 6 h, compared to 49.43% of EYA4-depleted cells (raw data can be found in FIG. 26A-C). EYA4-depleted cells resumed/finished S with a 2 h delay, and 78.3% of depleted cells entered G2/M 8 h post-release, showing that EYA4 depletion extends S-phase and delays cell division. Without wishing to be bound by theory, the most logical explanations for such observations are defects in DNA replication and aberrant checkpoint signals. Since EYA4 depletion appears to halt the cell cycle in the transition between S-phase and G2, we evaluated the activation of several proteins involved in G1 checkpoint (G1/S transition) and G2 checkpoint (schematic in FIG. 27C). The G1/S transition was examined to assess if the cells can initiate DNA replication. For this, the expression of cyclin E1, its partner CDK2, and its corresponding CDK inhibitors, p21^{WAF1/CIP1} and p27^{KIP1} (FIG. 20E) were determined. After double thymidine block (G1/S transition), synchronized EYA4 depleted cells appeared to accumulate p21^{WAF1/CIP1} and p27^{KIP1} especially EYA4 depleted with shRNA3. However, CDK2 does not seem to be affected by the CDK inhibitors, since the level of expression appears to be similar between control and EYA4 silenced cells. Cyclin E1 levels increase sharply in late G1, where it interacts and activates CDK2 allowing G1/S transition, then decrease in S-phase (Mazumder S, et al. (2004) Current Cancer Drug Targets 4(1): 65-75), as observed in control cells, but not in EYA4-depleted cells. This correlates with the accumulation of cells in G1-S at 6 h observed in cells depleted for EYA4 (FIG. 20D). Cyclin E1/CDK2 is an important part of the G1 checkpoint and deregulation in the G1/S transition may impair normal DNA replication, causing replication stress and DNA damage (Fagundes R, and Teixeira LK. (2021) Frontiers in Cell and Developmental Biology 9: 774845). Nevertheless, EYA4 silenced cells appear to be able to overcome the G1 checkpoint and initiate DNA replication with little or no delay. Upon release from the thymidine block, EYA4-depleted cells, especially shRNA1, exhibited a notable delay in S-phase compared to the EV control (FIG. 20D). This was confirmed by the accumulation of cyclin A (highly expressed in S-phase, decreasing in G2) for up to 10 hours post-release (FIG. 20E). Altogether, these data indicate that in the absence of EYA4, S-phase and its subsequent transition into G2 become prolonged. Without wishing to be bound by theory, these effects could stem from faulty DNA replication and/or the accumulation of DNA damage during S-phase.

### g. SPONTANEOUS REPLICATION STRESS IS OBSERVED IN THE ABSENCE OF EYA4

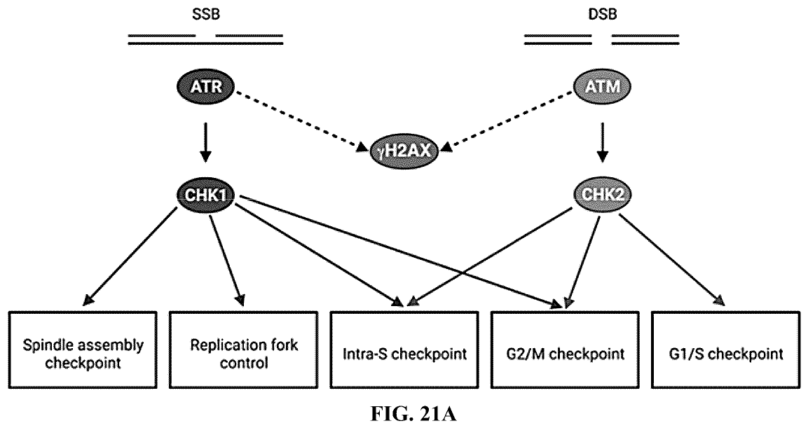

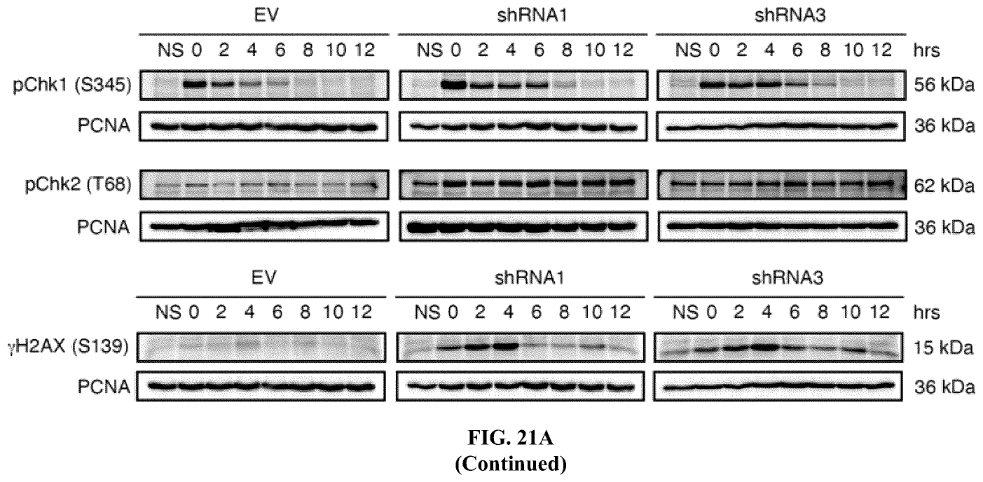

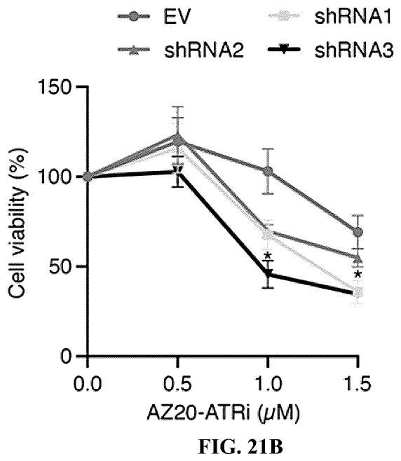

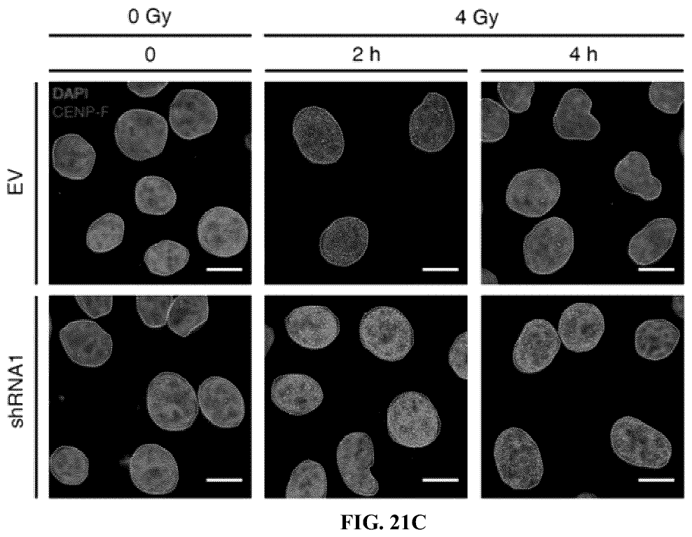

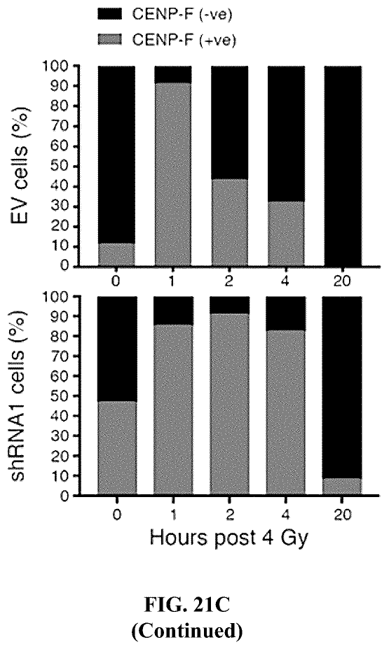

Since EYA4-depleted cells transition through G1/S and enter DNA replication, but S-phase appears to be longer and the S-G2 transition halted, the level of expression of pChk1 (S345) and pChk2 (T68) by immunoblotting was evaluated, to assess if the cells have accumulated spontaneous damaged DNA. To do so, cells were arrested in early S-phase with a double thymidine block. Checkpoint kinase 1 (Chk1) is a key player of DNA-damage-activated checkpoint response that acts downstream of ATR (Ataxia Telangiectasia and Rad3 related) kinase, in response to the formation of single-stranded DNA due to DNA damage of blocked replication forks (FIG. 21A). It is activated by all known forms of DNA damage, particularly triggering the intra-S- and G2/M-phase checkpoints (Qiu Z, et al. (2018) Radiotherapy and Oncology 126(3): 450-64). Chk2 is a stable protein expressed throughout the cell cycle. In response to DNA double-strand breaks, Chk2 becomes rapidly phosphorylated at threonine 68 by ATM (Ataxia Telangiectasia Mutated) (FIG. 21A). The kinase activity of Chk2 depends on the severity of DNA damage (Ward I M, et al. (2001) Journal of Biological Chemistry 276: 47755-8). Under normal conditions, EYA4-depleted cells accumulated pChk1 (S345) up to 8 h after release (FIG. 21A), but not the control, implying that replication fork stalling occurs in the absence of EYA4, and its resolution becomes delayed. Additionally, pChk2 (T68) is highly expressed in the absence of EYA4 (FIG. 21A), which, without wishing to be bound by theory, suggests the accumulation of double-stranded breaks (DSBs) that might be a consequence of replication fork collapse. Spontaneous accumulation of DNA damage was confirmed by evaluating the expression of the phosphorylated histone variant H2AX (S319, γH2AX) in early S-phase. Accumulation of γH2AX was observed in EYA4-depleted cells (FIG. 21A), but not in the control, indicating the presence of replication stress, which probably triggers the phosphorylation of H2AX on S139 by ATR (FIG. 21A). In accordance with these results, cells depleted for EYA4 were also found sensitive to AZ20, an ATR inhibitor (FIG. 21B). Since longer S-phase and halted cell cycle observed in the absence of EYA4 might be due to accumulation of replication stress, the assessment as to if the cells are able to progress throughout the cell cycle upon DNA damage induction was sought. The formation of CENP-F foci was followed after 4 Gy γ-irradiation, to identify S-phase and G2/M. CENP-F gradually accumulates during the cell cycle until it reaches peak levels in G2/M phases (weakly positive in S-phase), where it first associates with kinetochores in late G2 (Liao H, et al. (1995) Journal of Cell Biology 130(3): 507-18). Control cells accumulate in S-G2/M after γ-irradiation, indicating that the cell cycle is halted (1 h after 4 Gy) but they progress once DNA damage is resolved. Nevertheless, in the absence of EYA4, accumulation of CENP-F was observed up to 4 h after irradiation, indicating that the cells are taking longer to resolve DNA damage (FIG. 21C).

### h. EYA4 CONTRIBUTES TO HU RESISTANCE

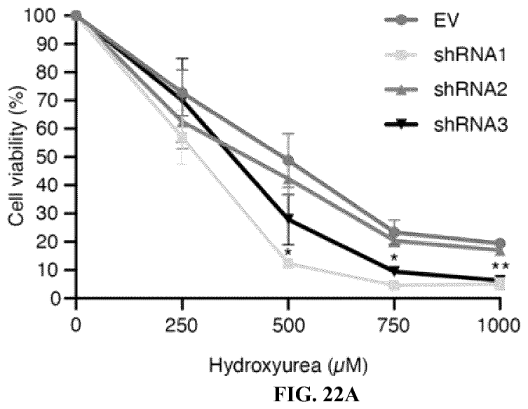

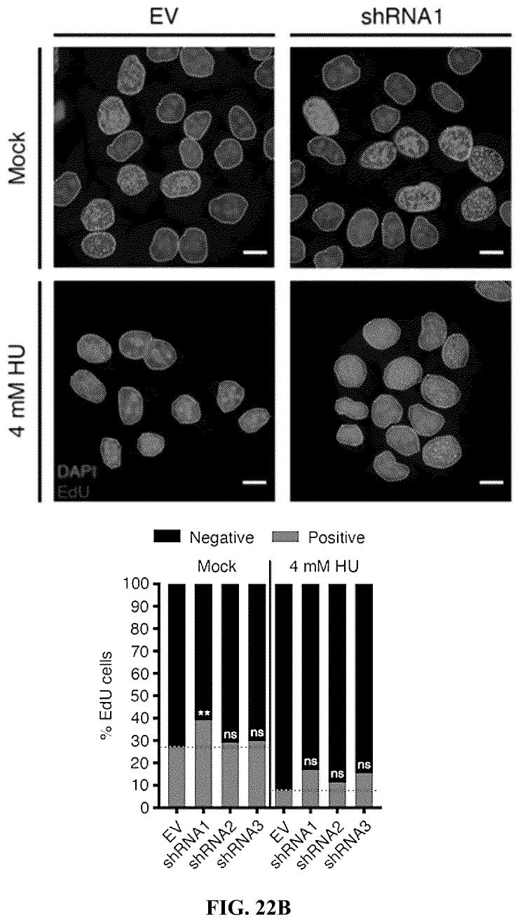

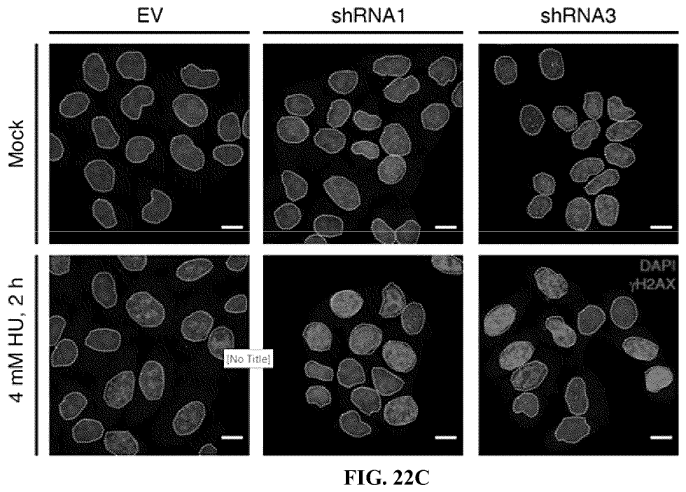

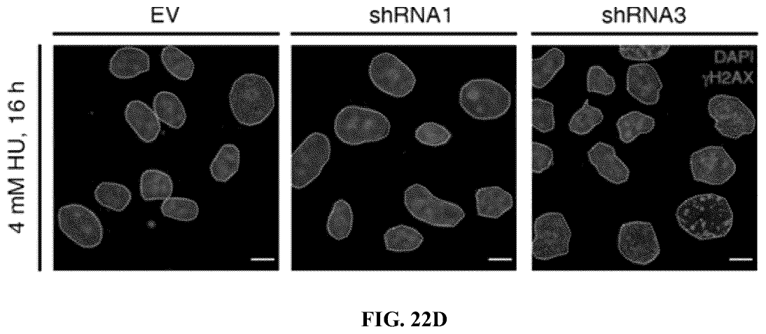

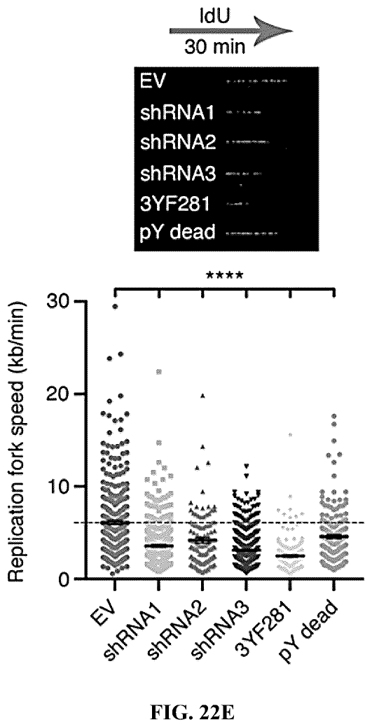

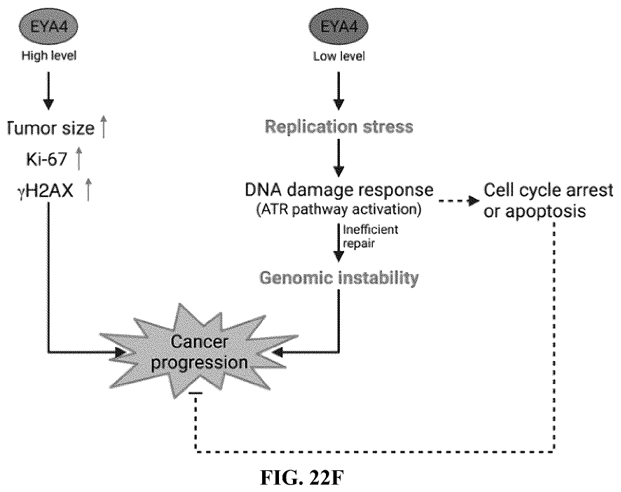

To address the potential role of EYA4 in the cellular response to replication stress, the effects of knocking down EYA4 were examined on the sensitivity to hydroxyurea (HU), which causes replication stress by depleting the intracellular pool of deoxynucleotides (Musialek M W, and Rybaczek D. (2021) Genes (Basel) 12(7): 1096). In accordance with the accumulation of replication stress and checkpoint activation, cells depleted for EYA4 were found to be sensitive to hydroxyurea in an MTT assay (FIG. 22A). In order to monitor DNA synthesis, cells were treated with 4 mM hydroxyurea for 2 h and then measured 5-ethynyl-2′-deoxyuridine (EdU) incorporation after the removal of HU. Under these conditions, silencing EYA4 resulted in a slightly increased rate of EdU incorporation (FIG. 22B), indicating that EYA4 might be involved in maintaining replication fork stability since EYA4-depleted cells appear to overcome the HU blockage and resume synthesis.

### i. EYA4 DEPLETION RESULTS IN INCREASED AND UNRESOLVED LEVELS of HU-Induced DSBs

Replication fork collapse resulting from chronic HU exposure generates double-stranded breaks (Musialek M W, and Rybaczek D. (2021) Genes (Basel) 12(7): 1096), which are rapidly marked by γH2AX. To examine the possible role of EYA4 in the repair of HU-induced DSBs, HeLa cells were incubated with 4 mM HU for 2 h and then allowed to recover for 2 h in the absence of the drug. Even though EYA4-depleted cells have high levels of endogenous DNA damage, an increase in HU-induced DSBs was observed in the absence of EYA4 (FIG. 22C). Next, HeLa cells were incubated with 4 mM HU for 16 h and then released for 18 h, to assess for unresolved DNA damage in the absence of EYA4. Although residual γH2AX foci were present in HeLa control cells after recovery from HU exposure, ˜10% more cells with >10 γH2AX foci per cell (FIG. 22D) were observed in the absence of EYA4, implying that these cells have a diminished ability to resolve HU-induced DNA damage. Without wishing to be bound by theory, these results together suggest that EYA4 contributes to replication-associated DNA damage repair.

### j. EYA4 IMPACTS REPLICATION FORK SPEED

To investigate the functional role of EYA4 in DNA replication, single-molecule analysis of replicated DNA fibers were utilized to test if the increased DSBs in EYA4-deficient cells affected replication fork (RF) progression and speed. It was found that the replication tracts are much shorter in EYA4 deficient cells compared to control cells, under normal conditions (FIG. 22E), demonstrating that genome-wide RF progression is strongly impaired by EYA4 depletion. Interestingly, the fork slowing observed was even more dramatic in the 3YF281 mutant cells, but not in the pY dead cells, showing that the serine/threonine phosphatase activity of EYA4 is essential for replication fork progression.

### k. DISCUSSION

EYA protein phosphatases have been associated with cancer pathologies, and they exhibit characteristics of oncogenic and tumor-suppressive activities depending on the tissue of origin. Because EYA are protein phosphatases, without wishing to be bound by theory, it is expected that lack of phosphorylation would impact a variety of cellular pathways depending on the protein substrates expressed and targeted in specific tissues. In this study, we sought to gain a better understanding of the cellular processes impacted by EYA4 deregulation in cancer, and specifically understand the possible role of EYA4 in breast carcinogenesis.

As previously reported the EYA4 gene is hypermethylated in the first intron-exon junction (Stirzaker C, et al. (2015) Nature Communications 6: 5899), and possibly over-expressed in triple-negative breast cancer patients, which correlates with publicly available TCGA dataset that shows amplification as the most common alteration in breast cancer patients. In this study provided herein, HeLa and breast cancer cell lines were used to investigate the proliferation rates of cells knocked down or over-expressing EYA4, ex vivo and as xenografts in small animals. While EYA4 is often not expressed in normal breast, it was found that MDA-MB-231 over-express EYA4, and are depending on its expression for survival. Over-expression of EYA4 was shown to drive the growth of ER⁺ primary tumors, and promote metastasis to distant organs such as lungs and livers. In triple-negative breast cancer xenografts, the knockdown of EYA4 was able to efficiently limit the spread of metastasis and the overall cancer burden. These two xenograft studies suggest that EYA4 therapeutic targeting is an interesting avenue that should be pursued for anti-breast cancer drug development. Over-expressing EYA4 in cancer could be used to predict patient outcomes and drug response.

EYA4 level is inversely correlated with ER status, with high expression largely found in triple-negative breast cancer, while ER⁺ tumors and cell lines express little or no EYA4. This warrants further investigation to fully understand the connection between Eyes Absent phosphatases and the hormonal status of cancerous tissues. In breast cancer, it is well-established that estrogen is a major driver of breast tumor growth through its role in cell proliferation, as well as an effective therapeutic target. It has been proposed that in MCF-7 cells, ER-α induces cell proliferation by regulating the cell cycle by stimulating the expression of PCNA and Ki-67 and suppressing of p53/p21 transcription (Liao X—H, et al. (2014) FEBS Journal 281: 927-42). The data provided herein shows that EYA4-depleted cells exhibit slower division rates as measured by live imaging. Further investigation by live imaging and the FUCCI system demonstrated that cells silenced for EYA4 undergo slower DNA synthesis, halting cell cycle progression, and undergoing endoreplication as a result of missed mitosis initiation. The data provided herein confirm previous observations in glioma where EYA4 over-expression promotes cell proliferation by directly suppressing the expression of p27^{KIP1}, suggesting p27^{KIP1} as a transcriptional target of EYA4 (Li Z, et al. (2018) Cellular Physiology and Biochemistry 49: 1856-69).

In EYA4-depleted cells, cell cycle arrest and DNA damage response (DDR) activation were observed. EYA4, and specifically the serine/threonine domain of EYA4, was shown to play an important novel role in replication fork progression. Several human diseases have been associated with defects in replication stress signaling, including Bloom syndrome (Shastri V M, et al. (2021) Nucleic Acids Research 49(15): 8699-713), Fanconi anemia (Michl J, et al. (2016) Nature Structural and Molecular Biology 23:755-7), Seckel syndrome (Murga M, et al. (2009) Nature Genetics 41(8): 891-8), Werner syndrome (Mukherjee S, et al. (2018) International Journal of Molecular Sciences 19(11): 3442), and the most common one, cancer (Gaillard H, et al. (2015) Nature Reviews Cancer 5: 276-89). This is the first evidence implicating EYA4, or any member of the EYA family, in the resolution of DNA replication-induced DNA damage. These results highlight the need for further characterization of the roles of EYA proteins in the DDR and genomic integrity.

Importantly, the serine/threonine phosphatase activity of EYA4 has been shown that is important for breast cancer progression and metastasis, suggesting that targeting the EYA4 phosphatase activity could help devise new cancer treatments directed against primary tumors and distant metastasis.

### l. REFERENCES

J. M. Daley, P. Sung, 53BP1, BRCA1, and the choice between recombination and end joining at DNA double-strand breaks. Mol Cell Biol 34, 1380-1388 (2014).
T. T. Paull, Reconsidering pathway choice: a sequential model of mammalian DNA double-strand break pathway decisions. Curr Opin Genet Dev 71, 55-62 (2021).
D. Wang et al., ATM-phosphorylated SPOP contributes to 53BP1 exclusion from chromatin during DNA replication. Sci Adv 7 (2021).
Z. Mirman, T. de Lange, 53BP1: a DSB escort. Genes Dev 34, 7-23 (2020).
K. Rothkamm et al., DNA damage foci: Meaning and significance. Environ Mol Mutagen 56, 491-504 (2015).
P. Moudry et al., TOPBP1 regulates RAD51 phosphorylation and chromatin loading and determines PARP inhibitor sensitivity. J Cell Biol 212, 281-288 (2016).
T. Clouaire et al., Comprehensive Mapping of Histone Modifications at DNA Double-Strand Breaks Deciphers Repair Pathway Chromatin Signatures. Mol Cell 72, 250-262 e256 (2018).
I. Revet et al., Functional relevance of the histone gammaH2Ax in the response to DNA damaging agents. Proc Natl Acad Sci USA 108, 8663-8667 (2011).
J. A. Brown, J. K. Eykelenboom, N. F. Lowndes, Co-mutation of histone H2AX S139A with Y142A rescues Y142A-induced ionising radiation sensitivity. FEBS Open Bio 2, 313-317 (2012).
P. J. Cook et al., Tyrosine dephosphorylation of H2AX modulates apoptosis and survival decisions. Nature 458, 591-596 (2009).
R. S. Hegde, K. Roychoudhury, R. N. Pandey, The multi-functional eyes absent proteins. Crit Rev Biochem Mol Biol 55, 372-385 (2020).
T. Ishino et al., Identification of a Novel Copy Number Variation of EYA4 Causing Autosomal Dominant Non-syndromic Hearing Loss. Otol Neurotol 42, e866-e874 (2021).
M. Morin et al., Insights into the pathophysiology of DFNA10 hearing loss associated with novel EYA4 variants. Sci Rep 10, 6213 (2020).
J. Shinagawa et al., Prevalence and clinical features of hearing loss caused by EYA4 variants. Sci Rep 10, 3662 (2020).
S. Ahmadmehrabi et al., Genome-first approach to rare EYA4 variants and cardio-auditory phenotypes in adults. Hum Genet 140, 957-967 (2021).
Y. Mi et al., Early truncation of the N-terminal variable region of EYA4 gene causes dominant hearing loss without cardiac phenotype. Mol Genet Genomic Med 9, e1569 (2021).
I. M. Wilson et al., EYA4 is inactivated biallelically at a high frequency in sporadic lung cancer and is associated with familial lung cancer risk. Oncogene 33, 4464-4473 (2014).
R. Towle, D. Truong, C. Garnis, Epigenetic mediated silencing of EYA4 contributes to tumorigenesis in oral dysplastic cells. Genes Chromosomes Cancer 55, 568-576 (2016).
I. Rebay, Multiple Functions of the Eya Phosphotyrosine Phosphatase. Mol Cell Biol 36, 668-677 (2015).
H. Zhou, L. Zhang, R. L. Vartuli, H. L. Ford, R. Zhao, The Eya phosphatase: Its unique role in cancer. Int J Biochem Cell Biol 96, 165-170 (2018).
B. Yuan et al., A phosphotyrosine switch determines the antitumor activity of ERbeta. The Journal of clinical investigation 124, 3378-3390 (2014).
H. L. Klein, The consequences of Rad51 overexpression for normal and tumor cells. DNA Repair (Amst) 7, 686-693 (2008).
Y. Feng, D. Wang, L. Xiong, G. Zhen, J. Tan, Predictive value of RAD51 on the survival and drug responsiveness of ovarian cancer. Cancer Cell Int 21, 249 (2021).
F. F. Depreux et al., Eya4-deficient mice are a model for heritable otitis media. The Journal of clinical investigation 118, 651-658 (2008).
Y. Cai et al., Experimental and computational framework for a dynamic protein atlas of human cell division. Nature 561, 411-415 (2018).
D. P. Nusinow et al., Quantitative Proteomics of the Cancer Cell Line Encyclopedia. Cell 180, 387-402 e316 (2020).
S. L. Carter, A. C. Eklund, I. S. Kohane, L. N. Harris, Z. Szallasi, A signature of chromosomal instability inferred from gene expression profiles predicts clinical outcome in multiple human cancers. Nat Genet 38, 1043-1048 (2006).
D. Chowdhury et al., A PP4-phosphatase complex dephosphorylates gamma-H2AX generated during DNA replication. Mol Cell 31, 33-46 (2008).
H. Cha et al., Wip1 directly dephosphorylates gamma-H2AX and attenuates the DNA damage response. Cancer Res 70, 4112-4122 (2010).
A. A. Hatimy, Brown, M. J. G., Flaus, A., Sweet, S. M. M., Histone H2AX Y142 phosphorylation is a low abundance modification. International Journal of Mass Spectrometry 391, 139-145 (2016).
S. Solier, Y. Pommier, The apoptotic ring: a novel entity with phosphorylated histones H2AX and H2B and activated DNA damage response kinases. Cell Cycle 8, 1853-1859 (2009).
Y. Okabe, T. Sano, S. Nagata, Regulation of the innate immune response by threonine-phosphatase of Eyes absent. Nature 460, 520-524 (2009).
Y. Okabe, T. Sano, S. Nagata, Regulation of the innate immune response by threonine-phosphatase of Eyes absent. Nature 460, 520-524 (2009).
M. J. Schellenberg, R. M. Petrovich, C. C. Malone, R. S. Williams, Selectable high-yield recombinant protein production in human cells using a GFP/YFP nanobody affinity support. Protein Sci 27, 1083-1092 (2018).
P. Caron et al., Non-redundant Functions of ATM and DNA-PKcs in Response to DNA Double-Strand Breaks. Cell Rep 13, 1598-1609 (2015).
J. O'Sullivan, S. Y. Mersaoui, G. Poirier, J. Y. Masson, Assessment of Global DNA Double-Strand End Resection using BrdU-DNA Labeling coupled with Cell Cycle Discrimination Imaging. J Vis Exp 10.3791/62553 (2021).
R. Tropee et al., The SWI/SNF subunit SMARCD3 regulates cell cycle progression and predicts survival outcome in ER+breast cancer. Breast Cancer Res Treat 185, 601-614 (2021).
A. J. Pierce, R. D. Johnson, L. H. Thompson, M. Jasin, XRCC3 promotes homology-directed repair of DNA damage in mammalian cells. Genes & Development 13, 2633-2638 (1999).
K. Nakanishi, F. Cavallo, E. Brunet, M. Jasin, Homologous recombination assay for interstrand cross-link repair. Methods in Molecular Biology 745, 283-291 (2011).
G. Peng et al., Genome-wide transcriptome profiling of homologous recombination DNA repair. Nat Commun 5, 3361 (2014).
M. Popova et al., Detection of c-Abl kinase-promoted phosphorylation of Rad51 by specific antibodies reveals that Y54 phosphorylation is dependent on that of Y315. FEBS Letters 583, 1867-1872 (2009).
S. Subramanyam, M. Ismail, I. Bhattacharya, M. Spies, Tyrosine phosphorylation stimulates activity of human RAD51 recombinase through altered nucleoprotein filament dynamics. PNAS 113, E6045-E6054 (2016).
B. Alligand, M. Le Breton, D. Marquis, F. Vallette, F. Fleury, Functional effects of diphosphomimetic mutations at cAbl-mediated phosphorylation sites on Rad51 recombinase activity. Biochimie 139 (2017).
P. B. Narayanaswamy, S. Tkachuk, H. Haller, I. Dumler, Y. Kiyan, CHK1 and RAD51 activation after DNA damage is regulated via urokinase receptor/TLR4 signaling. Cell Death Dis 7, e2383 (2016).
M. Reuter et al., BRCA2 diffuses as oligomeric clusters with RAD51 and changes mobility after DNA damage in live cells. J Cell Biol 207, 599-613 (2014).
F. Prado, Non-Recombinogenic Functions of Rad51, BRCA2, and Rad52 in DNA Damage Tolerance. Genes (Basel) 12 (2021).
M. J. Cabello-Lobato et al., Physical interactions between MCM and Rad51 facilitate replication fork lesion bypass and ssDNA gap filling by non-recombinogenic functions. Cell Rep 36, 109440 (2021).
M. Tarsounas, D. Davies, S. C. West, BRCA2-dependent and independent formation of RAD51 nuclear foci. Oncogene 22, 1115-1123 (2003).
T. Takenaka et al., Combined evaluation of Rad51 and ERCC1 expressions for sensitivity to platinum agents in non-small cell lung cancer. Int J Cancer 121, 895-900 (2007).
H. Maacke et al., Over-expression of wild-type Rad51 correlates with histological grading of invasive ductal breast cancer. Int J Cancer 88, 907-913 (2000).
M. M. Hoppe et al., Quantitative imaging of RAD51 expression as a marker of platinum resistance in ovarian cancer. EMBO Mol Med 13, e13366 (2021).
C. Wiese et al., Promotion of homologous recombination and genomic stability by RAD51AP1 via RAD51 recombinase enhancement. Mol Cell 28, 482-490 (2007).
B. de la Pe5a Avalos, E. Dray, Visualization of DNA Repair Proteins Interaction by Immunofluorescence. Journal of Visualized Experiments 160, e61447 (2020).
M. H. Dunlop et al., RAD51-associated protein 1 (RAD51AP1) interacts with the meiotic recombinase DMC1 through a conserved motif. J Biol Chem 286, 37328-37334 (2011).
E. Dray et al., Molecular basis for enhancement of the meiotic DMC1 recombinase by RAD51 associated protein 1 (RAD51AP1). Proc Natl Acad Sci USA 108, 3560-3565 (2011).
E. Dray et al., Enhancement of RAD51 recombinase activity by the tumor suppressor PALB2. Nat Struct Mol Biol 17, 1255-1259 (2010).
M. J. Schellenberg, R. M. Petrovich, C. C. Malone, R. S. Williams, Selectable high-yield recombinant protein production in human cells using a GFP/YFP nanobody affinity support. Protein Sci 27, 1083-1092 (2018).
C. Wiese et al., Promotion of homologous recombination and genomic stability by RAD51AP1 via RAD51 recombinase enhancement. Mol Cell 28, 482-490 (2007).
R. Prakash et al., Yeast Mph1 helicase dissociates Rad51-made D-loops: implications for crossover control in mitotic recombination. Genes Dev 23, 67-79 (2009).
Kong D, Ma W, Zhang D, Cui Q, Wang K, Tang J, et al. EYA1 promotes cell migration and tumor metastasis in hepatocellular carcinoma. American Journal of Translational Research. 2019; 11(4):2328-38.
Xu H, Jiao Y, Yi M, Zhao W, Wu K. EYA2 correlates with clinico-pathological features of breast cancer, promotes tumor proliferation, and predicts poor survival. Frontiers in Oncology. 2019; 9(26).
Zhu J, Hu L-B, Zhao Y—P, Zhang Y-Q. Prognostic role of EYA4 in lower grade glioma with IDH1 mutation and 1p19q co-deletion. World Neurosurgery.
2021; 149:e1174-e9.
Tootle T L, Silver S J, Davies E L, Newman V, Latek R R, Mills I A, et al. The transcription factor Eyes absent is a protein tyrosine phosphatase. Nature. 2003; 426:299-302.
Okabe Y, Sano T, Nagata S. Regulation of the innate immune response by threonine-phosphatase of Eyes absent. Nature. 2009; 460:520-4.
Rebay I. Multiple functions of the Eya phosphotyrosine phosphatase. Molecular and Cellular Biology. 2016; 36(5):668-77.
Morin M, Borreguero L, Booth K T, Lachgar M, Huygen P, Villamar M, et al. Insights into the pathophysiology of DFNA10 hearing loss associated with novel EYA4 variants. Scientific Reports. 2020; 6213.
Ahamadmehrabi S, Li B, Park J, Devkota B, Vujkovic M, Ko Y-A, et al. Genome-first approach to rare EYA4 variants and cardio-auditory phenotypes in adults. Human Genetics. 2021; 140:957-67.
Miller S J, Lan Z D, Hardiman A, Wu J, Kordich J J, Patmore D M, et al. Inhibition of Eyes Absent Homolog 4 expression induces malignant peripheral nerve sheath tumor necrosis. Oncogene. 2010; 29(3):368-79.
Zou H, Osborn N K, Harrington J J, Klatt K K, Molina J R, Burgart L J, et al. Frequent methylation of Eyes Absent 4 gene in Barrett's esophagus and esophageal adenocarcinoma. Cancer Epidemiology, Biomarkers and Prevention. 2005; 14(4):830-4.
Luo M, Li Y, Shi X, Yang W, Zhou F, Sun N, et al. Aberrant methylation of EYA4 promotes epithelial-mesenchymal transition in esophageal squamous cell carcinoma. Cancer Science. 2018; 109(6):1811-24.
Hou X, Peng J-X, Hao X-Y, Cai J-P, Liang L-J, Zhai J-M, et al. DNA methylation profiling identifies EYA4 gene as a prognostic molecular marker in hepatocellular carcinoma. Annals of Surgical Oncology. 2014; 21(12):3891-9.
Kim S-J, Tae C H, Hong S N, Min B—H, Chang D K, Rhee P-L, et al. EYA4 acts as a new tumor suppressor gene in colorectal cancer. Molecular Carcinogenesis. 2015; 54(12):1748-57.
Stirzaker C, Zotenko E, Song J Z, Qu W, Nair S S, Locke W J, et al. Methylome sequencing in triple-negative breast cancer reveals distinct methylation clusters with prognostic value. Nature Communications. 2015; 6:5899.
Lang L, Schnittger A. Endoreplication, a means to an end in cell growth and stress response. Science Direct. 2020; 54:85-92.
Matsuda M, Iwata Y, Koizumi N, Mishiba K-i. DNA double-strand breaks promote endoreduplication in radish cotyledon. Plant Cell Reports. 2018; 37:913-21.
Shu Z, Row S, Deng W-M. Endoreplication: The Good, the Bad, and the Ugly. Trends in Cell Biology. 2018; 28(6):465-74.
Sakaue-Sawano A, Kurokawa H, Morimura T, Hanyu A, Hama H, Osawa H, et al. Visualizing spatiotemporal dynamics of multicellular cell-cycle progression. Cell. 2008; 132(3):487-98.
Carceles-Cordon M, Rodriguez-Fernandez I, Rodriguez-Bravo V, Cordon-Cardo C, Domingo-Domenech J. In vivo bioluminescence imaging of luciferase-labeled cancer cells. Bio-Protocol. 2016; 6(6).
Thomas C, Gustafsson J-A. The different roles of ER subtypes in cancer biology and therapy. Nature Reviews. 2011; 11:597-608.
Liao X—H, Lu D-L, Wang N, Liu L-Y, Wang Y, Li Y-Q, et al. Estrogen receptor alpha mediates proliferation of breast cancer MCF-7 cells via a p21/PCNA/E2F1-dependent pathway. FEBS Journal. 2014; 281:927-42.
Cheon H, Wang Y, Wightman S M, Jackson M W, Stark G R. How cancer cells make and respond to interferon-I. Trends in Cancer. 2023; 9(1):83-92.
Qiu J, Xu J, Yao G, Zhu F, Wang Y, Fu Y. An ovarian large-cell neuroendocrine carcinoma accompanied by clear cell carcinoma with specific high level of AFP: Case report and review of the literature. Cancer Management and Research. 2022; 14:2235-41.
Zhou X, Zhou M, Zheng M, Tian S, Yang X, Ning Y, et al. Polyploid giant cancer cells and cancer progression. Frontiers in Cell and Developmental Biology. 2022; 10.
Hanahan D. Hallmarks of cancer: New dimensions. Cancer Discovery. 2022; 12(1):31-46.
Li Z, Qiu R, Qiu X, Tian T. EYA2 promotes lung cancer cell proliferation by downregulating the expression of PTEN. Oncotarget. 2017; 8(67):110837-48.
Lee H O, Davidson J M, Duronio R J. Endoreplication: polyploidy with purpose. Genes & Development. 2009; 23:2461-77.
Tagal V, Roth M G. Loss of Aurora kinase signaling allows lung cancer cells to adopt endoreplication and form polyploid giant cancer cells that resist antimitotic drugs. Cancer Research. 2021; 81(2):400-13.
Gandarillas A, Molinuevo R, Sanz-Gómez N. Mammalian endoreplication emerges to reveal a potential developmental timer. Cell death & Differentiation. 2018; 25:471-6.
Mazumder S, DuPree E L, Almasan A. A dual role of cyclin E in cell proliferation and apotosis may provide a target for cancer therapy. Current Cancer Drug Targets. 2004; 4(1):65-75.
Fagundes R, Teixeira L K. Cyclin E/CDK2: DNA replication, replication stress and genomic instability. Frontiers in Cell and Developmental Biology. 2021; 9:774845.
Qiu Z, Oleinick N L, Zhang J. ATR/CHK1 inhibitors and cancer therapy. Radiotherapy and Oncology. 2018; 126(3):450-64.
Ward I M, Wu X, Chen J. Threonine 68 of Chk2 is phosphorylated at sites of DNA strand breaks. Journal of Biological Chemistry. 2001; 276:47755-8.
Liao H, Winkfein R, Mack G, Rattner J B, Yen T J. CENP-F is a protein of the nuclear matrix that assembles onto kinetochores at late G2 and is rapidly degraded after mitosis. Journal of Cell Biology. 1995; 130(3):507-18.
Musialek M W, Rybaczek D. Hydroxyurea—The Good, the Bad and the Ugly. Genes (Basel). 2021; 12(7):1096.
Li Z, Qiu R, Qiu X, Tian T. EYA4 promotes cell proliferation through downregulation of p27Kip1 in glioma. Cellular Physiology and Biochemistry. 2018; 49:1856-69.
Shastri V M, Subramanian V, Schmidt K H. A novel cell-cycle-regulated interation of the Bloom syndrome helicase BLM with Mcm6 controls replication-linked processes. Nucleic Acids Research. 2021; 49(15):8699-713.
Michl J, Zimmer J, Buffa F M, McDermott U, Tarsounas M. FANCD2 limits replication stress and genome instability in cells lacking BRCA2. Nature Structural and Molecular Biology. 2016; 23:755-7.
Murga M, Bunting S, Monta5a MF, Soria R, Mulero F, Cañamero M, et al. A mouse model of the ATR-Seckel syndrome reveals that replicative stress during embryogenesis limits mammalian lifespan. Nature Genetics. 2009; 41(8):891-8.
Mukherjee S, Sinha D, Bhattacharya S, Srinivasan K, Abdisalaam S, Asaithamby A. Werner syndrome protein and DNA replication. International Journal of Molecular Sciences. 2018; 19(11):3442.
Gaillard H, Garcia-Muse T, Aguilera A. Replication stress and cancer. Nature Reviews Cancer. 2015; 5:276-89.
Collins I, Garrett M D. Preclinical profiles and contexts for CHK1 and CHK2 inhibitors. Humana Press, Chain. 2018.

### 5. EYA Proteins and EWS are Essential for Genomic Stability

### a. Ewing Sarcoma (ES) is a Deadly Pediatric Malignancy

Sarcomas arise in all age groups but disproportionally affect pediatric and young adult patients. 200 children and teenagers are diagnosed each year with Ewing tumors, a fatal disease with limited therapeutic options. Surgery remains the main ES treatment. ES exhibits the characteristics of DNA repair deficient tumors, making it exquisitely sensitive to irradiation and genotoxic chemotherapy. Loss of Ewing sarcoma breakpoint region 1 protein (EWSR1) leads to mitotic and meiotic defects and sterility, suggesting a possible role in homologous recombination (HR), chromosome pairing and segregation (Damm, E. and Odenthal-Hesse, L. (2023) Curr Top Dev Biol, 151, 27-42; Tian, H., et al., (2021) Mol Biol Cell, 32, 1-14; Park, H., et al., (2021) J Biol Chem, 296, 1001641-3). While effective, chemotherapy is highly toxic, and radiotherapy must be avoided whenever possible in children due to elevated risks of secondary cancers. The fusion protein EWS::FLI1 commonly found in EwS has proven an elusive target and drug development efforts are focusing instead on its modulators. Targeting DNA repair deficiencies in cells containing EWSR1 rearrangements could prove a valid therapeutic avenue.

### b. EYA Proteins and EWS are Essential for Genomic Stability

Protein phosphatases EYA1 and EYA4 have been extensively characterized from the Eyes Absent (EYA) family which are non-typical proteins with dual phosphatase: serine/threonine and tyrosine activities (Tootle, T. L. et al., (2003) Nature, 426, 299-3026; Okabe, Y., et al., (2009) Nature, 460, 520-5247). EYAs dephosphorylate H2AX to prevent apoptosis and favour DNA repair (Wilson, I. M., et al., (2014) Oncogene, 33, 4464-4473) and this was confirmed in ES-derived cell lines. Using recombination substrates in cells (Nakanishi, K., Cavallo, F., Brunet, E. and Jasin, M. (2011) Homologous recombination assay for interstrand cross-link repair. Methods Mol Biol, 745, 283-291; Bennardo, N., et al., (2008) PLoS Genet, 4, e1000110), It was found that EYA4 silencing severely decreases faithful DNA repair (HR) and increases the more error prone end-joining pathway cNHEJ, thus increasing chromosomal instability (CIN) and genome rearrangements, which are hallmark of ES. Interestingly, we have identified that EYA proteins and EWSR1 belong to the same protein network using reciprocal BioID and also found in vivo interactions by immunoprecipitation—mass spectrometry (IP-MS). Co-PI Bishop recently showed that EWSR1 promotes HR at double stranded breaks (Countryman, P., et al. (2018) J Biol Chem, 293, 1054-1069), but how EYA and EWSR1 interact to promote faithful repair and promote cancer avoidance in ES remains to be elucidated.

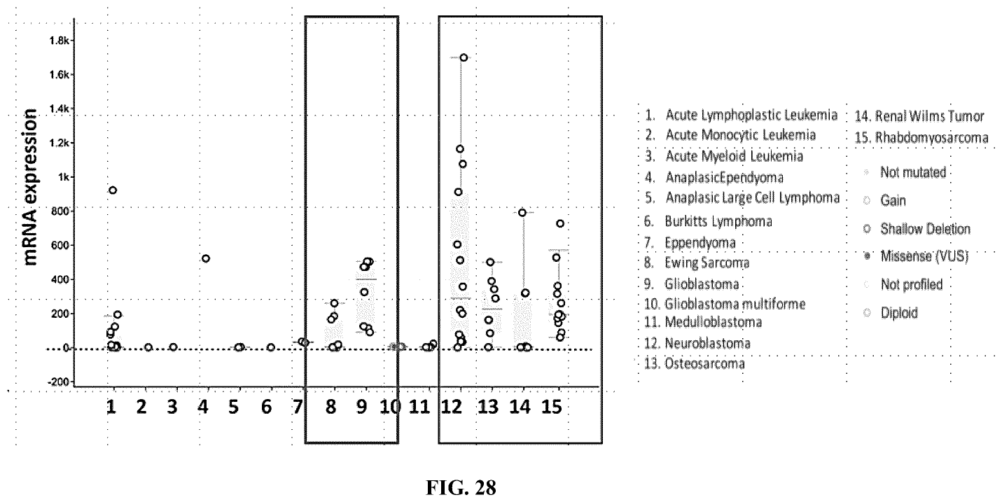

EYA protein phosphatases contain a variable region specific to each isoform, an EYA conserved domain, and two phosphatase domains that are extremely well conserved, between EYA1-4 and among vertebrates. This conservation hints at common phosphatase substrates, and a possible functional redundancy. However, EYA proteins are often tissue specific. In pediatric cancer, EYAs are mostly overexpressed in solid tumors. While EYA2 is rarely expressed except in eppendyoma and rhabdomyosarcoma, EYA1, EYA3 and EYA4 are expressed in most solid pediatric tumors including ES mRNA expression of EYA4 cross pediatric tumor datasets obtained from pediatric cBioPortal shows elevated levels in specific subtypes of pediatric solid tumors (boxes) compared to basal level as calculated across healthy tissue (dotted line) (FIG. 28).

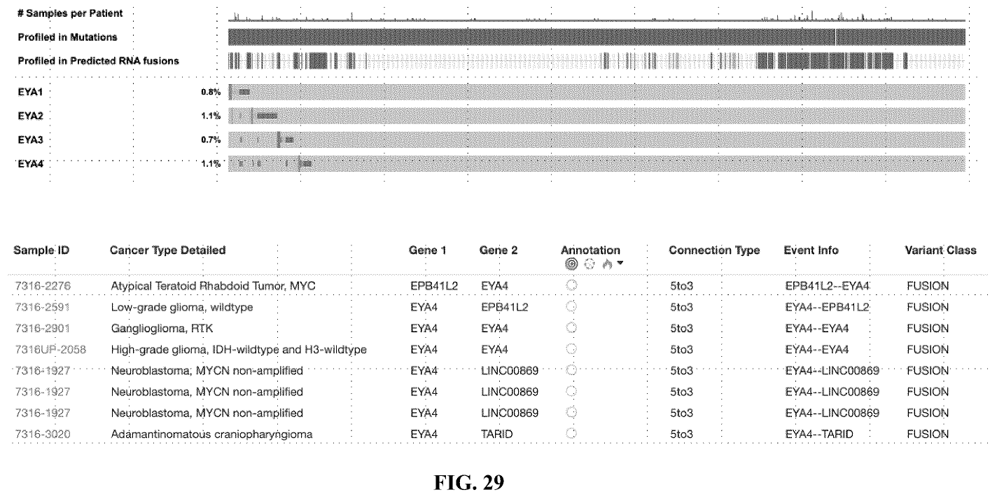

Interestingly, in pediatric cancer, EYA4 in tissue is often found as a fusion (FIG. 29). Fusions are known mechanisms of malignant transformation and support carcinogenesis (Chansky, H. A., et al. (2001) Cancer Research, 61, 3586-3590; Yang, L., Chansky, H. A. and Hickstein, D. D. (2000) J Biol Chem, 275, 37612-37618; Liu, Y., et al. (2023) Nat Commun 14, 1739, 12-14). Proteomic profiling identifies EYA3, EYA4 and EWS::FL1 interaction.

Proximity-dependent biotin identification (BioID) is a powerful tool for identifying novel protein-protein and proximity-based interactions in live cells (Le Sage, V., et al., (2016) Curr. Protoc. Cell Biol., 73, 17 19 11-17 19 12; Roux, K. J., et al., (2013) Curr Protoc Protein Sci, 74, Unit 19 23). BioID involves expression of the promiscuous mutant prokaryotic biotin ligase BirA fused to a protein of interest, which upon expression will biotinylate proximal endogenous proteins. BirA to EYA4 were fused and demonstrated targeted biotinylation following transient expression of BirA-EYA4. Fused BirA-EYA4 was expressed or a myc-BirA control in the HeLa and U2OS cell lines, stimulated BirA-mediated biotinylation by the addition of biotin and performed streptavidin pull-downs followed by liquid chromatography-mass spectrometry (LC-MS/MS) to identify EYA4-associated proteins. LC-MS/MS and quantitative label-free analysis was conducted and analysed at the proteomic core facility. EWS was found in the proteomic profile of EYA4-associated proteins in response to DDR pathway activation. The reverse, complementary experiment was then performed where EWS::FLI1 or EWS was tagged with TurboID, biotinylated and pulled down, in HEK293T cells (excerpt data in Table 6 and (Elzi, D. J., et al., (2014) J Proteome Res, 13, 3783-3791)). This confirmed that EYA3 and EYA4 are found in the vicinity of Ewing proteins, and possible interactors.

TABLE 6

| | | | | | | Cell |
|---|---|---|---|---|---|---|
| Accession | Gene | Log2FC | FC | P-value | Interactome | location |
| O95677 | EYA4 | 1.90823702 | 3.7535014 | 0.0003692 | EWS:FLI1 | Nuclear |
| O95677 | EYA4 | 2.40248067 | 5.28711485 | 0.0043932 | EWS:FLI1 | Cytoplasmic |
| Q99504 | EYA3 | 0.74100316 | 1.67133758 | 0.00158501 | EWS:FLI1 | Cytoplasmic |
| O95677 | EYA4 | 0.60768258 | 1.52380952 | 0.00010598 | EWS | Cytoplasmic |

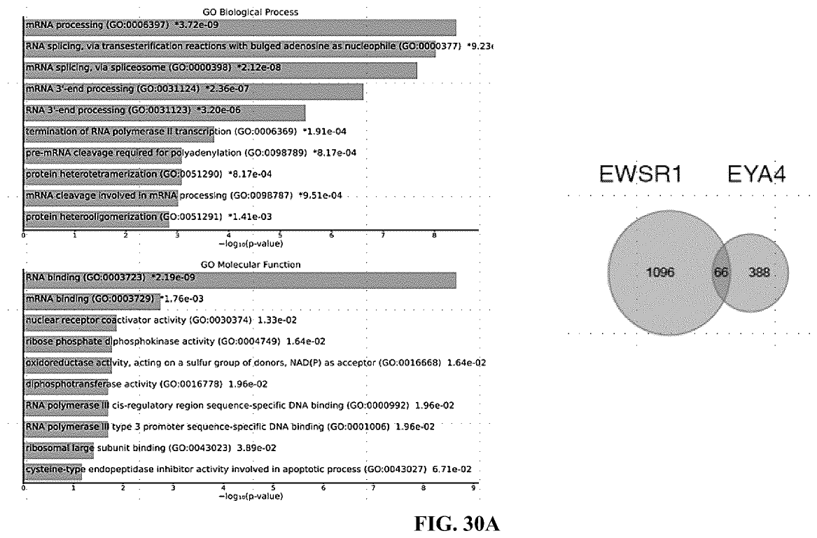

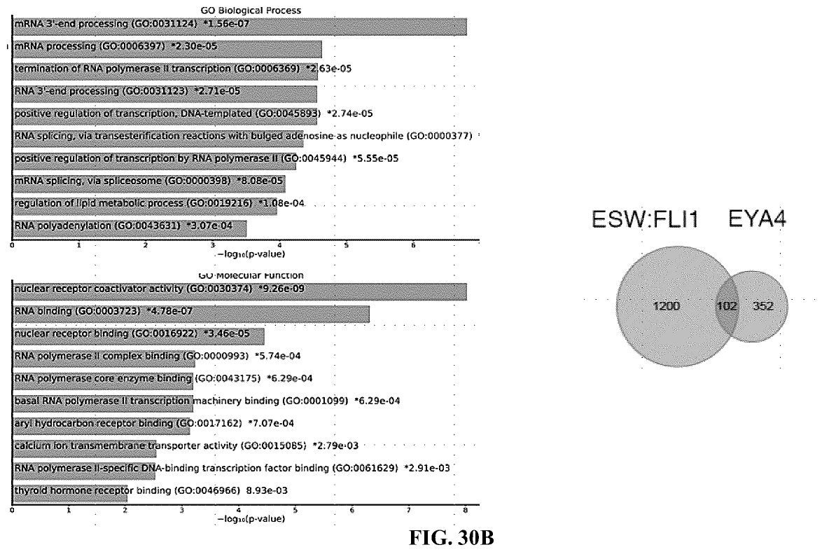

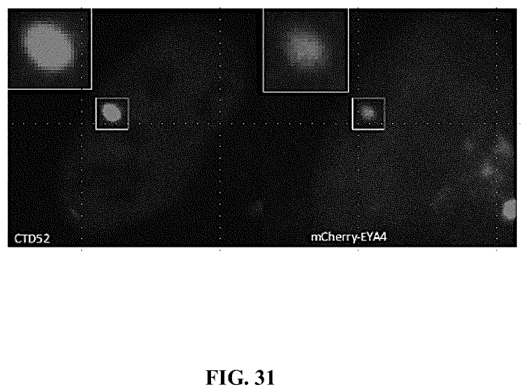

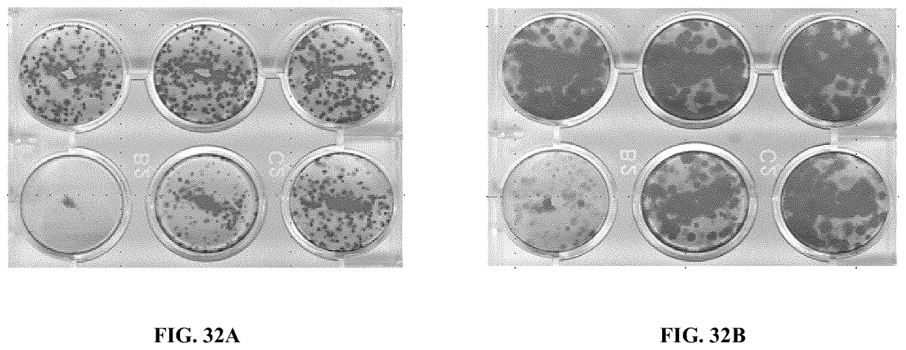

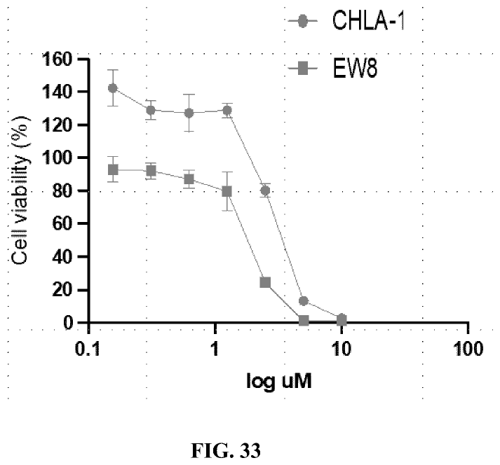

Interestingly, the DACH-SIX-PAX network, a known interactor of EYA proteins and transcription regulator, was also pulled down by the EWS::FLI1 construct, reinforcing the likelihood of a EYA-EWS complex. Proteins enriched in both the EYA4 and the EWS or EWS::FLI1 experiments largely overlap, with 66 genes shared between EYA4 and EWSR1 interactome (FIG. 30A), and 102 between EYA4 and EWS::FLI1 (FIG. 30B) indicating functional interactions. RNA metabolism proteins were strongly over-represented (FIG. 30A and FIG. 30A). IP-MS of EYA4 in various cell lines identified EWSR1 as possible direct interactor.

### c. Pancreatic Cancer (PC) is a Recalcitrant Cancer

Pancreatic cancer (PC) is not the most frequent cancer but continuing recalcitrance to therapy makes it the most lethal malignancy. Patients can be subclassified based on their HRD (Homologous Recombination Deficiency) status, and HRD+ patients often, but not always, carry a mutation in a major homologous recombination (HR) gene, such as BRCA1, BRCA2, or PALB2. Biomarkers of DNA Damage Repair (DDR) and HR in particular, are associated with response to PARP inhibitors and platinum-based therapies, and for this reason the genotype of patients has prognostic and therapeutic value. However, drug-resistance spontaneously occurs over time in a majority of patients, and they die from refractory diseases.

Previously conducted multi-omic studies were performed encompassing transcriptome, genome and proteome characterization, as well as molecular analysis of 61 novel PC patient-derived cell lines (PDCL) (Dreyer et al., (2021) Gastroenterology 160(1):362-377.e13). It was established that this cohort is faithful to the biology and drug responses of PC tumors, and that this panel is a valuable resource for drug development, as it can help predict drug response in sub-populations. While HRD confers exquisite sensitivity to cytotoxic chemotherapies, radiation, and inhibitors of poly (ADP-ribose) polymerase (PARPi), an enzyme essential for the repair of single stranded DNA breaks (SSB), together, we were able to identify a novel signature of replication stress, which predicts response to ATR and WEEl inhibitors. The establishment of distinct signatures that only partially overlap allows us to test various molecules highly beneficial to patients, regardless of their HRD status, or after HR has been restored, such as in the case of acquired resistance to PARP inhibition or platinum therapy.

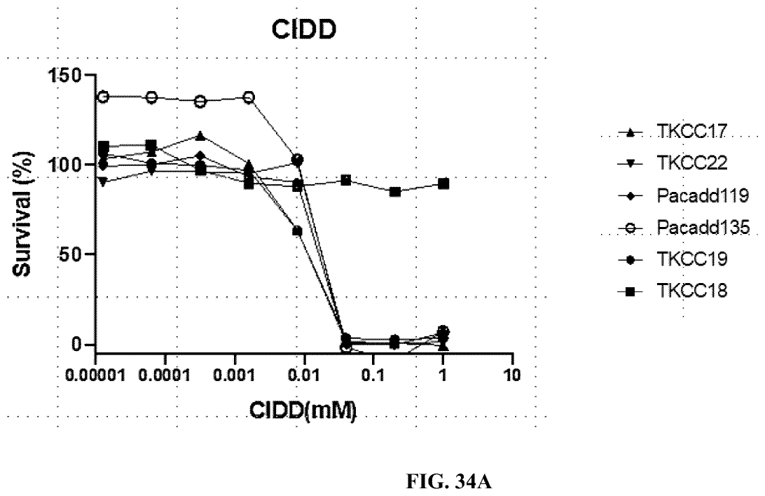

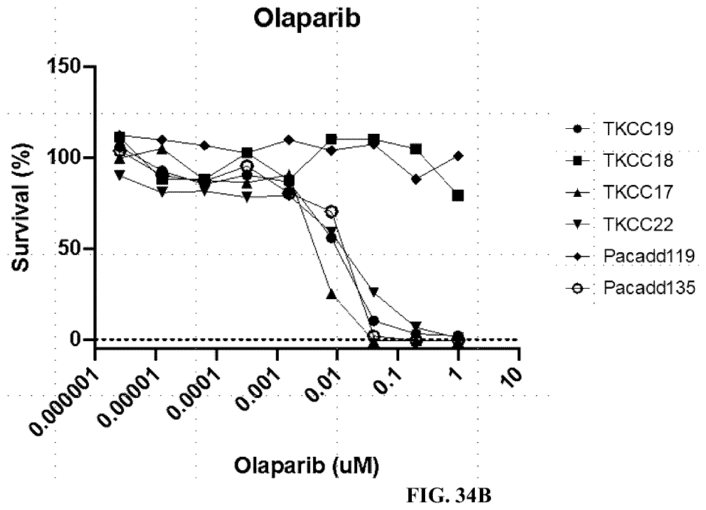

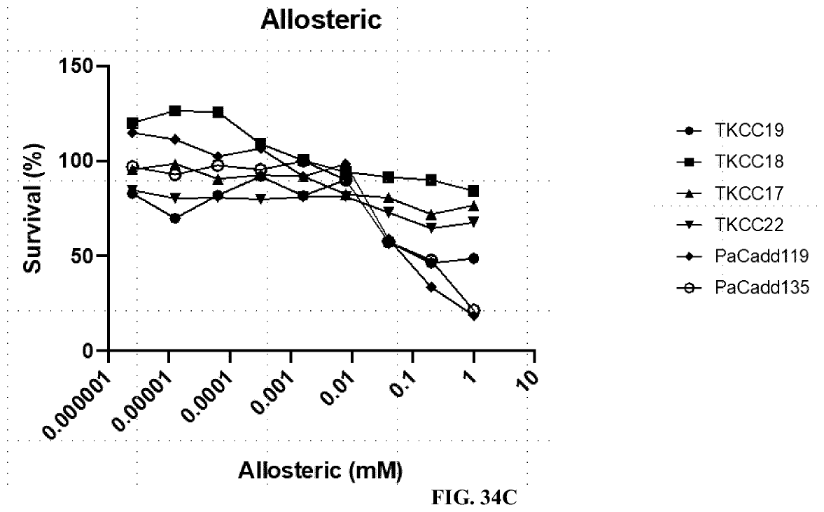

Using molecules that target regulators of DNA metabolism mechanisms (repair, recombination, replication), such as kinase and phosphatase can offer a therapeutic window for drug resistant, HRD>0 patients. EYA4 inhibitors have been tested on the pancreatic cell lines panel. EYA4i efficacy correlates perfectly with the HRD status of the cells, and efficiently kills cells that are HRD+(FIG. 34A) as a monotherapy, even more efficiently than the PARPi Olaparib (FIG. 34B). A published allosteric inhibitor that is known to inhibit EYA3 (CSCS14817788384, 3-chloro-N′-{[5-(pyrimidin-2-ylsulfanyl)furan-2-yl]methylidene}benzohydrazide; slide 2 top right) was used as a control and does not efficiently inhibit growth of pancreatic cells or enhances killing at the concentrations tested (FIG. 34C). EYA4i have therapeutic potential for the treatment of pancreatic cancer, including PARPi refractory disease.

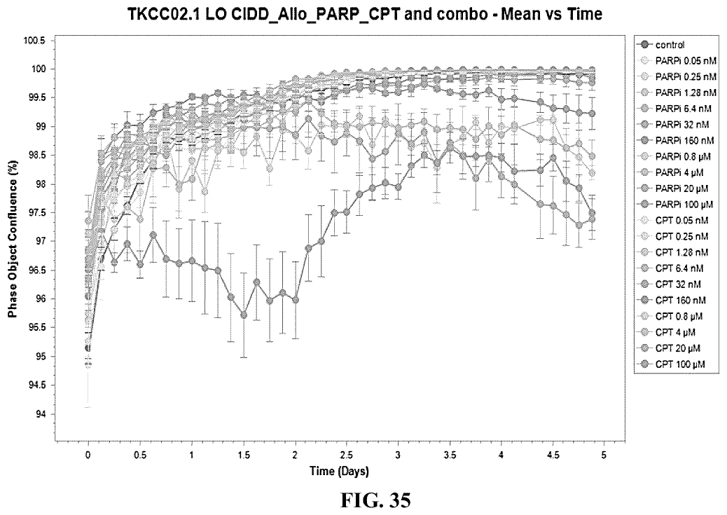

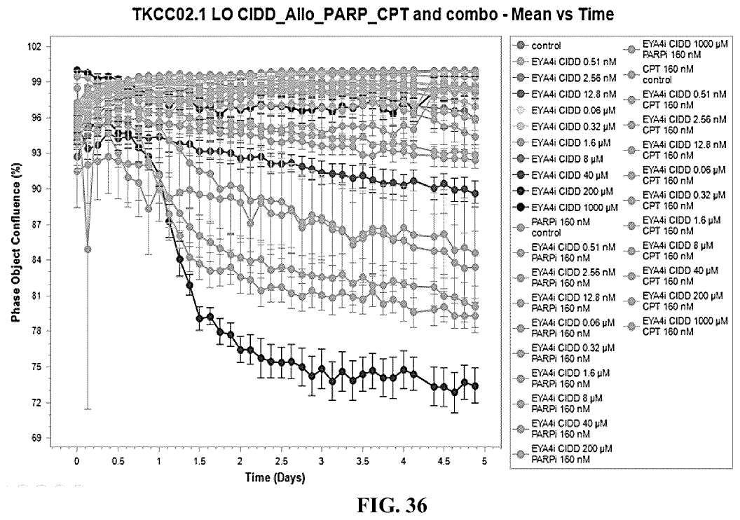

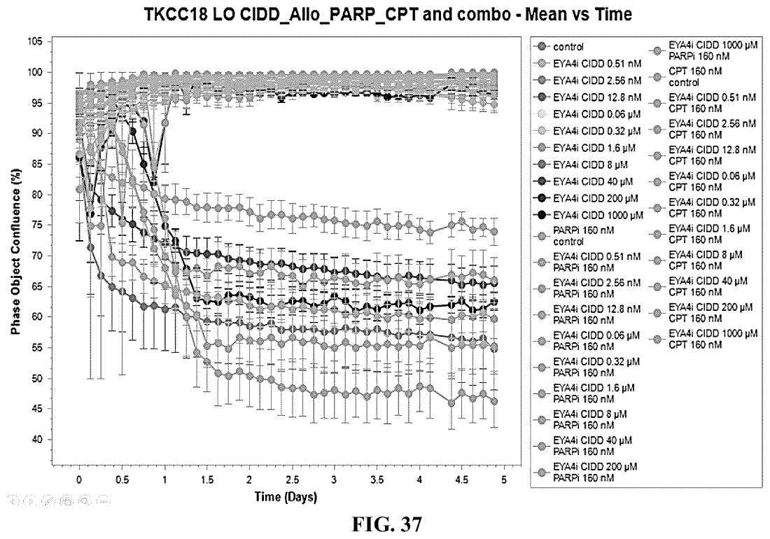

Similar doses of EYA4i were tested alone and in combination with PARPi (olaparib) or Camptothecin (CPT). At doses tested, neither PARPi nor CPT are lethal to TKCC02.1 (FIG. 35). However, EYA4i is efficient as single agent but also sensitizes cells to replication poisons in both TKCC02.1 (FIG. 36) and TKCC18 (FIG. 37). Without wishing to be bound by theory, this could be an indication for irinotecan combination with an EYA4 inhibitor.

### d. References

Damm, E. and Odenthal-Hesse, L. (2023) Orchestrating recombination initiation in mice and men. Curr Top Dev Biol, 151, 27-42.
Tian, H., Billings, T. and Petkov, P. M. (2021) EWSR1 affects PRDM9-dependent histone 3 methylation and provides a link between recombination hotspots and the chromosome axis protein REC8. Mol Biol Cell, 32, 1-14.
Park, H., Kim, H., Hassebroek, V., Azuma, Y., Slawson, C. and Azuma, M. (2021) Chromosomal localization of Ewing sarcoma EWSR1/FLI1 protein promotes the induction of aneuploidy. J Biol Chem, 296, 100164.
Christopher B. Nelson, S. R., Kaushik Roychoudhury, Yaw Sing Tan, Caroline J. Atkinson, Alexander P. Sobinoff, Christopher G. Tomlinson, Anton Hsu, Robert Lu, Eloise Dray, Michelle Haber, Jamie I. Fletcher, Anthony J. Cesare, Rashmi S. Hegde, Hilda A. Pickett. (2022) The Eyes Absent family members EYA4 and EYA1 promote PLK1 activation and successful mitosis through tyrosine dephosphorylation. Biorxiv.
Bárbara de la Peña Avalos, R. T., Pascal H. G. Duijf, Eloïse Dray. (2023) EYA4 drives breast cancer progression and metastasis through its novel role in replication stress avoidance. Under review at Molecular Cancer, Preprinted on Research Square.
Tootle, T. L., Silver, S. J., Davies, E. L., Newman, V., Latek, R. R., Mills, I. A., Selengut, J. D., Parlikar, B. E. and Rebay, I. (2003) The transcription factor Eyes absent is a protein tyrosine phosphatase. Nature, 426, 299-302.
Okabe, Y., Sano, T. and Nagata, S. (2009) Regulation of the innate immune response by threonine-phosphatase of Eyes absent. Nature, 460, 520-524.
Wilson, I. M., Vucic, E. A., Enfield, K. S., Thu, K. L., Zhang, Y. A., Chari, R., Lockwood, W. W., Radulovich, N., Starczynowski, D. T., Banath, J. P. et al. (2014) EYA4 is inactivated biallelically at a high frequency in sporadic lung cancer and is associated with familial lung cancer risk. Oncogene, 33, 4464-4473.
Nakanishi, K., Cavallo, F., Brunet, E. and Jasin, M. (2011) Homologous recombination assay for interstrand cross-link repair. Methods Mol Biol, 745, 283-291.
Bennardo, N., Cheng, A., Huang, N. and Stark, J. M. (2008) Alternative-NHEJ is a mechanistically distinct pathway of mammalian chromosome break repair. PLoS Genet, 4, e1000110.
Countryman, P., Fan, Y., Gorthi, A., Pan, H., Strickland, J., Kaur, P., Wang, X., Lin, J., Lei, X., White, C. et al. (2018) Cohesin S A2 is a sequence-independent DNA-binding protein that recognizes DNA replication and repair intermediates. The Journal of biological chemistry, 293, 1054-1069.
Chansky, H. A., Hu, M., Hickstein, D. D. and Yang, L. (2001) Oncogenic TLS/ERG and EWS/Fli-1 fusion proteins inhibit RNA splicing mediated by YB-1 protein. Cancer research, 61, 3586-3590.
Yang, L., Chansky, H. A. and Hickstein, D. D. (2000) EWS. Fli-1 fusion protein interacts with hyperphosphorylated RNA polymerase II and interferes with serine-arginine protein-mediated RNA splicing. The Journal of biological chemistry, 275, 37612-37618.
Liu, Y., Klein, J., Bajpai, R. et al. Etiology of oncogenic fusions in 5,190 childhood cancers and its clinical and therapeutic implication. Nat Commun 14, 1739 (2023).
Le Sage, V., Cinti, A. and Mouland, A. J. (2016) Proximity-Dependent Biotinylation for Identification of Interacting Proteins. Curr. Protoc. Cell Biol., 73, 17 19 11-17 19 12.
Roux, K. J., Kim, D. I. and Burke, B. (2013) BioID: a screen for protein-protein interactions. Curr Protoc Protein Sci, 74, Unit 19 23.
Elzi, D. J., Song, M., Hakala, K., Weintraub, S. T. and Shiio, Y. (2014) Proteomic Analysis of the EWS-Fli-1 Interactome Reveals the Role of the Lysosome in EWS-Fli-1 Turnover. J Proteome Res, 13, 3783-3791.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of treating a disorder associated with overexpression of an eyes absent (EYA) protein in a subject in need thereof, the method comprising administering to the subject an effective amount of a compound having a structure represented by a formula: 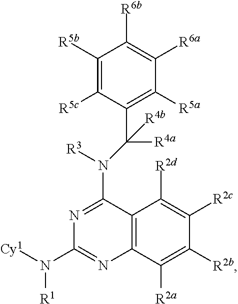
wherein R¹ is selected from hydrogen and C1-C4 alkyl;
wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein R³ is selected from hydrogen and C1-C4 alkyl;
wherein each of R^{4a} and R^{4b} is independently selected from hydrogen, halogen, and C1-C4 alkyl;
wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein each of R^{6a} and R^{6b} is independently selected from hydrogen and halogen; and
wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl,
provided that at least one of R^{6a} and R^{6b} is halogen,
or a pharmaceutically acceptable salt thereof, wherein the disorder is a vascular disease, a fibrosis-related disorder, hearing loss, or a metabolic disease.

2. The method of claim 1, wherein the disorder is associated with overexpression of eyes absent homolog 4 (EYA4) or eyes absent homolog 2 (EYA2).

3. The method of claim 1, wherein R^{6a} is halogen.

4. The method of claim 3, provided that either:
(a) R^{6b} is chloro;
(b) R^{6b} is hydrogen; or
(c) R^{6b} is halogen and at least one of R^{5a}, R^{5b}, and R^{5c} is not hydrogen.

5. The method of claim 1, wherein R^{6a} is chloro.

6. The method of claim 1, wherein R^{6b} is halogen.

7. The method of claim 1, wherein Cy¹ is an unsubstituted C3-C8 cycloalkyl.

8. The method of claim 1, wherein the compound has a structure represented by a formula: 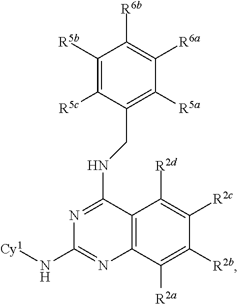
or a pharmaceutically acceptable salt thereof.

9. The method of claim 1, wherein the compound has a structure represented by a formula: 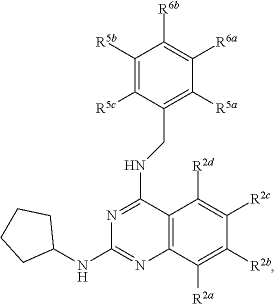
or a pharmaceutically acceptable salt thereof.

10. The method of claim 1, wherein the compound is a structure selected from: 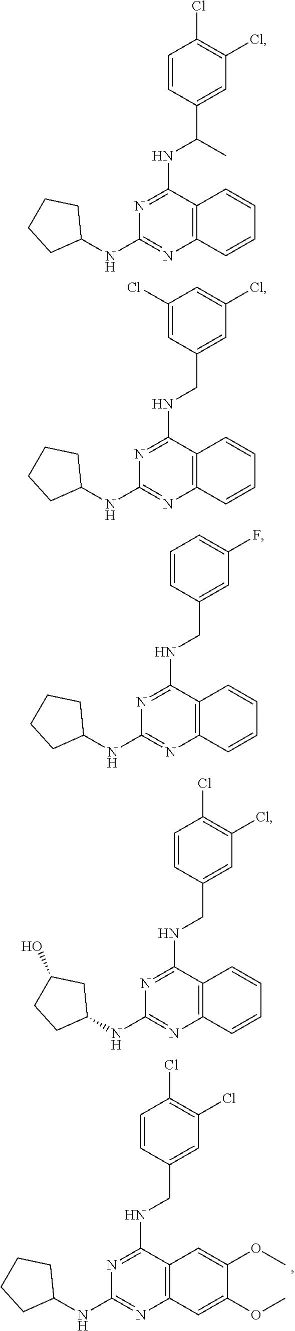 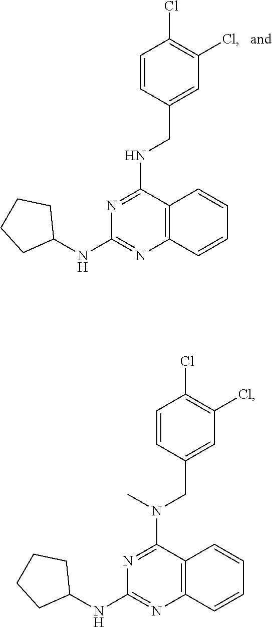 [SMILES: Clc1ccc(CNc2nc(NC3CCCC3)nc3ccccc23)cc1Cl ; CN(Cc1ccc(Cl)c(Cl)c1)c1nc(NC2CCCC2)nc2ccccc12]
or a pharmaceutically acceptable salt thereof.

11. The method of claim 1, wherein the compound is: 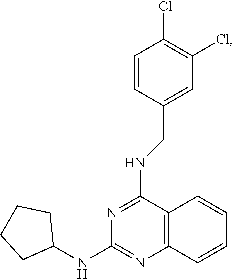 [SMILES: Clc1ccc(CNc2nc(NC3CCCC3)nc3ccccc23)cc1Cl]
or a pharmaceutically acceptable salt thereof.

12. The method of claim 1, wherein the disorder is a vascular disease.

13. The method of claim 1, wherein the disorder is a fibrosis-related disorder.

14. The method of claim 1, wherein the disorder is hearing loss.

15. The method of claim 1, wherein the disorder is a metabolic disease.

16. A method of treating cancer in a subject in need thereof, the method comprising administering to the subject a compound having a structure represented by a formula: 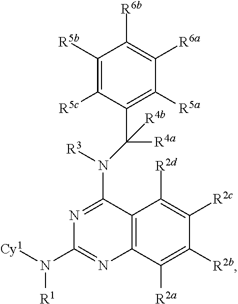
wherein R¹ is selected from hydrogen and C1-C4 alkyl;
wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein R³ is selected from hydrogen and C1-C4 alkyl;
wherein each of R^{4a} and R^{4b} is independently selected from hydrogen, halogen, and C1-C4 alkyl;
wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein each of R^{6a} and R^{6b} is independently selected from hydrogen and halogen; and
wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl,
provided that at least one of R^{6a} and R^{6b} is halogen,
or a pharmaceutically acceptable salt thereof, wherein the cancer comprises a tumor that overexpresses at least one eyes absent (EYA) protein.

17. The method of claim 16, wherein the tumor overexpresses EYA4.

18. The method of claim 16, wherein the tumor overexpresses EYA2.

19. The method of claim 16, wherein the compound has a structure represented by a formula: 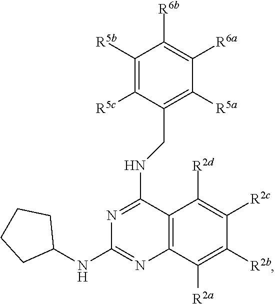
or a pharmaceutically acceptable salt thereof.

20. The method of claim 16, wherein the compound has a structure selected from: 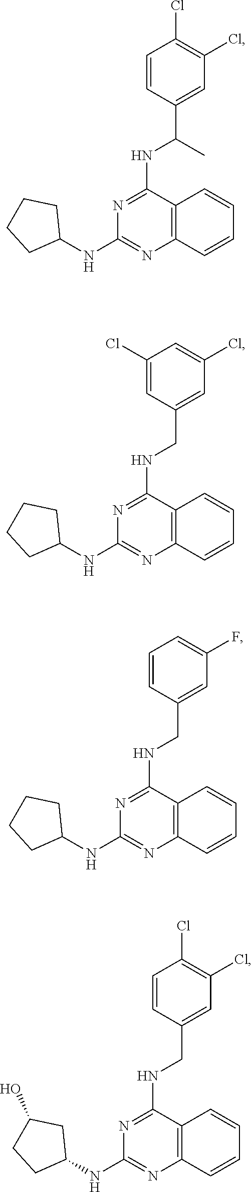 [SMILES: CC(Nc1nc(NC2CCCC2)nc2ccccc12)c1ccc(Cl)c(Cl)c1 ; Clc1cc(Cl)cc(CNc2nc(NC3CCCC3)nc3ccccc23)c1 ; Fc1cccc(CNc2nc(NC3CCCC3)nc3ccccc23)c1 ; O[C@H]1CC[C@@H](Nc2nc(NCc3ccc(Cl)c(Cl)c3)c3ccccc3n2)C1] 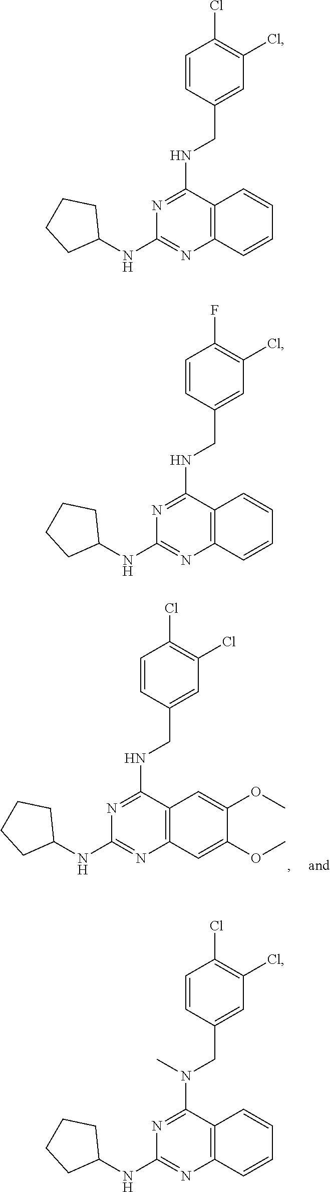 [SMILES: Clc1ccc(CNc2nc(NC3CCCC3)nc3ccccc23)cc1Cl ; Fc1ccc(CNc2nc(NC3CCCC3)nc3ccccc23)cc1Cl ; COc1cc2nc(NC3CCCC3)nc(NCc3ccc(Cl)c(Cl)c3)c2cc1OC ; CN(Cc1ccc(Cl)c(Cl)c1)c1nc(NC2CCCC2)nc2ccccc12]
or a pharmaceutically acceptable salt thereof.
